SEQUENCE LISTING (270 sequences)

SEQ ID NO: 1 - DNA, 360 bp, Artificial Sequence
  IgG1 Heavy Chain MSM R601
gaagttcaactgctggagtccggtggtggtctggtacagccgggtggttctctgcgtctgagttgcgcggccagtggctttaccttcagtagctatgccatgagctgggtgcgtcaggcgccgggcaaaggtctggaatgggttagcgcgattagcggtagtggcggtagcacgtactatgcggatagcgtgaaaggccgttttaccatttctcgcgacaacagcaagaacacgctgtacctgcagatgaactcactgcgtgccgaagatacggccatgtattactgtgcgagacatggctactccgctacaaggcagggactcgactactggggccagggaaccttggtcaccgtctcg

SEQ ID NO: 2 - DNA, 324 bp, Artificial Sequence
  IgG1 Light Chain MSM R601
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatattcttcttctcctctcacgttcggccaagggaccaaggtggaaatcaaa

SEQ ID NO: 3 - protein, 451 aa, Artificial Sequence
  IgG1 Heavy Chain MSM R601
EVQLLESGGGLVQPGGSLRLSCAASGFTFSSYAMSWVRQAPGKGLEWVSAISGSGGSTYYADSVKGRFTISRDNSKNTLYLQMNSLRAEDTAMYYCARHGYSATRQGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 215 aa, Artificial Sequence
  IgG1 Light Chain MSM R601
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 12 aa, Artificial Sequence
  IgG1 CDR1 Heavy Chain MSM R601
FTFSSYAMSWVR

SEQ ID NO: 6 - protein, 16 aa, Artificial Sequence
  IgG1 CDR2 Heavy Chain MSM R601
VSAISGSGGSTYYADS

SEQ ID NO: 7 - protein, 18 aa, Artificial Sequence
  IgG1 CDR3 Heavy Chain MSM R601
CARHGYSATRQGLDYWGQ

SEQ ID NO: 8 - protein, 12 aa, Artificial Sequence
  IgG1 CDR1 Light Chain MSM R601
RASQSVSSSYLA

SEQ ID NO: 9 - protein, 7 aa, Artificial Sequence
  IgG1 CDR2 Light Chain MSM R601
GASSRAT

SEQ ID NO: 10 - protein, 13 aa, Artificial Sequence
  CDR3 Light Chain MSM R601
CQQYSSSPLTFGQ

SEQ ID NO: 11 - DNA, 357 bp, Artificial Sequence
  IgG1 Heavy Chain MSM R602
gaagttcaactgctggagtccggtggtggtctggtacagccgggtggttctctgcgtctgagttgcgcggccagtggctttaccttcagtagctatgccatgagctgggtgcgtcaggcgccgggcaaaggtctggaatgggttagcgcgattagcggtagtggcggtagcacgtactatgcggatagcgtgaaaggccgttttaccatttctcgcgacaacagcaagaacacgctgtacctgcagatgaactcactgcgtgccgaagatacggccgtgtattactgtgcgagaaaaggctattacggaggtatggcaatggactactggggccagggaaccttggtcaccgtctcg

SEQ ID NO: 12 - DNA, 324 bp, Artificial Sequence; identical to SEQ ID NO: 2
  IgG1 Light Chain MSM R602

SEQ ID NO: 13 - protein, 450 aa, Artificial Sequence
  IgG1 Heavy Chain MSM R602
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARKGYYGGMAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 215 aa, Artificial Sequence; identical to SEQ ID NO: 4
  IgG1 Light Chain MSM R602

SEQ ID NO: 15 - protein, 12 aa, Artificial Sequence
  IgG1 CDR1 Heavy Chain MSM R602
FTFSSYAMSWVR

SEQ ID NO: 16 - protein, 16 aa, Artificial Sequence
  IgG1 CDR2 Heavy Chain MSM R602
VSAISGSGGSTYYADS

SEQ ID NO: 17 - protein, 17 aa, Artificial Sequence
  IgG1 CDR3 Heavy Chain MSM R602
CARKGYYGGMAMDYWGQ

SEQ ID NO: 18 - protein, 12 aa, Artificial Sequence
  IgG1 CDR1 Light Chain MSM R602
RASQSVSSSYLA

SEQ ID NO: 19 - protein, 7 aa, Artificial Sequence
  IgG1 CDR2 Light Chain MSM R602
GASSRAT

SEQ ID NO: 20 - protein, 13 aa, Artificial Sequence
  IgG1 CDR3 Light Chain MSM R602
CQQYSSSPLTFGQ

SEQ ID NO: 21 - DNA, 366 bp, Artificial Sequence
  IgG1 Heavy Chain MSM R604
gaagttcaactgctggagtccggtggtggtctggtacagccgggtggttctctgcgtctgagttgtgcggccagtggctttaccttcactaactattctatgcattgggtgcgtcaggctccgggcaaaggtctggaatgggttagcttgattagtccgtatagtagctcgacctactatgctgatagcatgaaaggccgttttaccatttctcgcgacaacagcaagaacacgctgtacctgcagatgaactcactgcgtgccgaagatacggccgtgtattactgtgcgagaggtcgttactacggtgcttactcttacccgggcttcgactactggggccagggaaccttggtcaccgtctcg

SEQ ID NO: 22 - DNA, 324 bp, Artificial Sequence
  IgG1 Light Chain MSM R604
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatcggctaagaatcctctcacgttcggccaagggaccaaggtggaaatcaaa

SEQ ID NO: 23 - protein, 453 aa, Artificial Sequence
  IgG1 Heavy Chain MSM R604
EVQLLESGGGLVQPGGSLRLSCAASGFTFTNYSMHWVRQAPGKGLEWVSLISPYSSSTYYADSMKGRFTISRDNSKNTLYLQMNSLRAEDTAVYYCARGRYYGAYSYPGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 215 aa, Artificial Sequence
  Light Chain MSM R604
EIVLTQSPGTLSLSPGERATLSCRASQSVSSSYLAWYQQKPGQAPRLLIYGASSRATGIPDRFSGSGSGTDFTLTISRLEPEDFAVYYCQQSAKNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 12 aa, Artificial Sequence
  IgG1 CDR1 Heavy Chain MSM R604
FTFTNYSMHWVR

SEQ ID NO: 26 - protein, 16 aa, Artificial Sequence
  IgG1 CDR2 Heavy Chain MSM R604
VSLISPYSSSTYYADS

SEQ ID NO: 27 - protein, 20 aa, Artificial Sequence
  IgG1 CDR3 Heavy Chain MSM R604
CARGRYYGAYSYPGFDYWGQ

SEQ ID NO: 28 - protein, 12 aa, Artificial Sequence
  IgG1 CDR1 Light Chain MSM R604
RASQSVSSSYLA

SEQ ID NO: 29 - protein, 7 aa, Artificial Sequence
  IgG1 CDR2 Light Chain MSM R604
GASSRAT

SEQ ID NO: 30 - protein, 13 aa, Artificial Sequence
  CDR3 Light Chain MSM R604
CQQSAKNPLTFGQ

SEQ ID NO: 31 - DNA, 366 bp, Artificial Sequence
  IgG1 Heavy Chain MSM R605
gaagttcaactgctggagtccggtggtggtctggtacagccgggtggttctctgcgtctgagttgcgcggccagtggctttaccttcagtagctatgccatgagctgggtgcgtcaggcgccgggcaaaggtctggaatgggttagcgcgattagcggtagtggcggtagcacgtactatgcggatagcgtgaaaggccgttttaccatttctcgcgacaacagcaagaacacgctgtacctgcagatgaactcactgcgtgccgaagatacggccgtgtattactgtgcgagaaactctatgaaaggtcagcgtaaacagcgtggcatggactactggggccagggaaccttggtcaccgtctcg

SEQ ID NO: 32 - DNA, 324 bp, Artificial Sequence
  IgG1 Light Chain MSM R605
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatctagggcgactcctctcacgttcggccaagggaccaaggtggaaatcaaa

SEQ ID NO: 33 - protein, 453 aa, Artificial Sequence
  IgG1 Heavy Chain MSM R605
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARNSMKGQRKQRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 215 aa, Artificial Sequence
  IgG1 Light Chain MSM R605
EIVLTQSPGTLSLSPGERATLSCRASQSVSSSYLAWYQQKPGQAPRLLIYGASSRATGIPDRFSGSGSGTDFTLTISRLEPEDFAVYYCQQSRATPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 12 aa, Artificial Sequence
  IgG1 CDR1 Heavy Chain MSM R605
FTFSSYAMSWVR

SEQ ID NO: 36 - protein, 16 aa, Artificial Sequence
  IgG1 CDR2 Heavy Chain MSM R605
VSAISGSGGSTYYADS

SEQ ID NO: 37 - protein, 20 aa, Artificial Sequence
  IgG1 CDR3 Heavy Chain MSM R605
CARNSMKGQRKQRGMDYWGQ

SEQ ID NO: 38 - protein, 12 aa, Artificial Sequence
  CDR1 Light Chain MSM R605
RASQSVSSSYLA

SEQ ID NO: 39 - protein, 7 aa, Artificial Sequence
  IgG1 CDR2 Light Chain MSM R605
GASSRAT

SEQ ID NO: 40 - protein, 13 aa, Artificial Sequence
  IgG1 CDR3 Light Chain MSM R605
CQQSRATPLTFGQ

SEQ ID NO: 41 - DNA, 363 bp, Artificial Sequence
  IgG1 Heavy Chain MSM R606
gaagttcaactgctggagtccggtggtggtctggtacagccgggtggttctctgcgtctgagttgtgcggccagtggctttaccttcagtaactattatatccattgggtgcgtcaggctccaggcaaaggtctggaatgggttagcattattaatccgtggagtggcaggacctactatgcggatagcgtgaaaggccgttttaccatttctcgcgacaacagcaagaacacgctgtacctgcagatgaactcactgcgtgccgaagatacggccgtgtattactgtgcgagaggcaggttgcggagcctctcttattccggcattgactactggggccagggaaccttggtcaccgtctcg

SEQ ID NO: 42 - DNA, 324 bp, Artificial Sequence
  IgG1 Light Chain MSM R606
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatcttcttattctcctatcacgttcggccaagggaccaaggtggaaatcaaa

SEQ ID NO: 43 - protein, 452 aa, Artificial Sequence
  IgG1 Heavy Chain MSM R606
EVQLLESGGGLVQPGGSLRLSCAASGFTFSNYYIHWVRQAPGKGLEWVSIINPWSGRTYYADSVKGRFTISRDNSKNTLYLQMNSLRAEDTAVYYCARGRLRSLSYSGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 215 aa, Artificial Sequence
  IgG1 Light Chain MSM R606
EIVLTQSPGTLSLSPGERATLSCRASQSVSSSYLAWYQQKPGQAPRLLIYGASSRATGIPDRFSGSGSGTDFTLTISRLEPEDFAVYYCQQSSYSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 12 aa, Artificial Sequence
  IgG1 CDR1 Heavy Chain MSM R606
FTFSNYYIHWVR

SEQ ID NO: 46 - protein, 16 aa, Artificial Sequence
  IgG1 CDR2 Heavy Chain MSM R606
VSIINPWSGRTYYADS

SEQ ID NO: 47 - protein, 19 aa, Artificial Sequence
  IgG1 CDR3 Heavy Chain MSM R606
CARGRLRSLSYSGIDYWGQ

SEQ ID NO: 48 - protein, 12 aa, Artificial Sequence
  IgG1 CDR1 Light Chain MSM R606
RASQSVSSSYLA

SEQ ID NO: 49 - protein, 7 aa, Artificial Sequence
  IgG1 CDR2 Light Chain MSM R606
GASSRAT

SEQ ID NO: 50 - protein, 13 aa, Artificial Sequence
  IgG1 CDR3 Light Chain MSM R606
CQQSSYSPITFGQ

SEQ ID NO: 51 - DNA, 366 bp, Artificial Sequence
  IgG1 Heavy Chain MSM R607
gaagttcaactgctggagtccggtggtggtctggtacagccgggtggttctctgcgtctgagttgtgcggccagtggctttaccttcagttcgtattatatgcattgggtgcgtcaggctccgggcaaaggtctggaatgggttagcttgattgctccggcgaatagcgatacctactatgcggatagcgtgaaaggccgttttaccatttctcgcgacaacagcaagaacacgctgtacctgcagatgaactcactgcgtgccgaagatacggccgtgtattactgtgcgagatcttcttacactcgtcattcttctaaacgtggcatggactactggggccagggaaccttggtcaccgtctcg

SEQ ID NO: 52 - DNA, 324 bp, Artificial Sequence
  IgG1 Light Chain MSM R607
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaagctgcttattctcctatcacgttcggccaagggaccaaggtggaaatcaaa

SEQ ID NO: 53 - protein, 453 aa, Artificial Sequence
  IgG1 Heavy Chain MSM R607
EVQLLESGGGLVQPGGSLRLSCAASGFTFSSYYMHWVRQAPGKGLEWVSLIAPANSDTYYADSVKGRFTISRDNSKNTLYLQMNSLRAEDTAVYYCARSSYTRHSSKRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 215 aa, Artificial Sequence
  IgG1 Light Chain MSM R607
EIVLTQSPGTLSLSPGERATLSCRASQSVSSSYLAWYQQKPGQAPRLLIYGASSRATGIPDRFSGSGSGTDFTLTISRLEPEDFAVYYCQQAAYSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 12 aa, Artificial Sequence
  IgG1 CDR1 Heavy Chain MSM R607
FTFSSYYMHWVR

SEQ ID NO: 56 - protein, 16 aa, Artificial Sequence
  IgG1 CDR2 Heavy Chain MSM R607
VSLIAPANSDTYYADS

SEQ ID NO: 57 - protein, 20 aa, Artificial Sequence
  IgG1 CDR3 Heavy Chain MSM R607
CARSSYTRHSSKRGMDYWGQ

SEQ ID NO: 58 - protein, 12 aa, Artificial Sequence
  IgG1 CDR1 Light Chain MSM R607
RASQSVSSSYLA

SEQ ID NO: 59 - protein, 7 aa, Artificial Sequence
  IgG1 CDR2 Light Chain MSM R607
GASSRAT

SEQ ID NO: 60 - protein, 13 aa, Artificial Sequence
  IgG1 CDR3 LIght Chain MSM R607
CQQAAYSPITFGQ

SEQ ID NO: 61 - DNA, 363 bp, Artificial Sequence
  IgG1 Heavy Chain MSM R608
gaagtgcaactggtacagtccggtgcggaagttaaaaagccgggttctagtgtcaaggtgtcatgcaaagccagtggcggcaccctcacttcttattatatctcttgggtgcgtcaggccccgggccaaggtctggaatggatgggttatatttctccgtcttctggctatacctattatgcgcagaaatttcaaggccgtgtgaccattactgctgatgaaagcacttctacagcctacatggagctgagttcactgcgctccgaggacacggccgtgtattactgtgcgagacgttaccgtggtcgtggttacgtttacggcttggactactggggccagggaaccctggtcaccgtctcg

SEQ ID NO: 62 - DNA, 324 bp, Artificial Sequence
  IgG1 Light Chain MSM R608
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatcttattatgctcctgtcacgttcggccaagggaccaaggtggaaatcaaa

SEQ ID NO: 63 - protein, 452 aa, Artificial Sequence
  Human HC AA MSM R608
EVQLVQSGAEVKKPGSSVKVSCKASGGTLTSYYISWVRQAPGQGLEWMGYISPSSGYTYYAQKFQGRVTITADESTSTAYMELSSLRSEDTAVYYCARRYRGRGYVYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 215 aa, Artificial Sequence
  IgG1 Light Chain MSM R608
EIVLTQSPGTLSLSPGERATLSCRASQSVSSSYLAWYQQKPGQAPRLLIYGASSRATGIPDRFSGSGSGTDFTLTISRLEPEDFAVYYCQQSYYAPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 12 aa, Artificial Sequence
  IgG1 CDR1 Heavy Chain MSM R608
GTLTSYYISWVR

SEQ ID NO: 66 - protein, 16 aa, Artificial Sequence
  IgG1 CDR2 Heavy Chain MSM R608
MGYISPSSGYTYYAQK

SEQ ID NO: 67 - protein, 19 aa, Artificial Sequence
  IgG1 CDR3 Heavy Chain MSM R608
CARRYRGRGYVYGLDYWGQ

SEQ ID NO: 68 - protein, 12 aa, Artificial Sequence
  IgG1 CDR1 Light Chain MSM R608
RASQSVSSSYLA

SEQ ID NO: 69 - protein, 7 aa, Artificial Sequence
  IgG1 CDR2 Light Chain MSM R608
GASSRAT

SEQ ID NO: 70 - protein, 13 aa, Artificial Sequence
  IgG1 CDR3 Light Chain MSM R608
CQQSYYAPVTFGQ

SEQ ID NO: 71 - DNA, 366 bp, Artificial Sequence
  IgG1 Heavy Chain MSM R609
gaagtgcaactgctggagtccggtggtggtctggtacagccgggtggttctctgcgtctgagttgtgcggccagtggctttaccttcactgcatcttatatgagctgggtgcgtcaggctccgggcaaaggtctggaatgggttagcaatatttctccgaagtatggcaatacctactatgcggatagcgtgaaaggccgttttaccatttctcgcgacaacagcaagaacacgctgtacctgcagatgaactcactgcgtgccgaagatacggccgtgtattactgtgcgagaagcatcaggagcgccaagtattcctattacgggattgactactggggccagggaaccctggtcaccgtctcg

SEQ ID NO: 72 - DNA, 324 bp, Artificial Sequence
  IgG1 Light Chain MSM R609
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatctgcttctgctcctgtcacgttcggccaagggaccaaggtggaaatcaaa

SEQ ID NO: 73 - protein, 453 aa, Artificial Sequence
  IgG1 Heavy Chain MSM R609
EVQLLESGGGLVQPGGSLRLSCAASGFTFTASYMSWVRQAPGKGLEWVSNISPKYGNTYYADSVKGRFTISRDNSKNTLYLQMNSLRAEDTAVYYCARSIRSAKYSYYGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 215 aa, Artificial Sequence
  IgG1 Light Chain MSM R609
EIVLTQSPGTLSLSPGERATLSCRASQSVSSSYLAWYQQKPGQAPRLLIYGASSRATGIPDRFSGSGSGTDFTLTISRLEPEDFAVYYCQQSASAPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 12 aa, Artificial Sequence
  IgG1 CDR1 Heavy Chain MSM R609
FTFTASYMSWVR

SEQ ID NO: 76 - protein, 16 aa, Artificial Sequence
  IgG1 CDR2 Heavy Chain MSM R609
VSNISPKYGNTYYADS

SEQ ID NO: 77 - protein, 20 aa, Artificial Sequence
  IgG1 CDR3 Heavy Chain MSM R609
CARSIRSAKYSYYGIDYWGQ

SEQ ID NO: 78 - protein, 12 aa, Artificial Sequence
  IgG1 CDR1 Light Chain MSM R609
RASQSVSSSYLA

SEQ ID NO: 79 - protein, 7 aa, Artificial Sequence
  IgG1 CDR2 Light Chain MSM R609
GASSRAT

SEQ ID NO: 80 - protein, 13 aa, Artificial Sequence
  IgG1 CDR3 Light Chain MSM R609
CQQSASAPVTFGQ

SEQ ID NO: 81 - DNA, 357 bp, Artificial Sequence
  IgG1 Heavy Chain MSM R610
gaagttcaactgctggagtccggtggtggtctggtacagccgggtggttctctgcgtctgagttgcgcggccagtggctttaccttcactggctcttctatgagctgggtgcgtcaggctccgggcaaaggtctggaatgggttagccttatttcgtcgaggggtagctatacctactatgcggatagcgtgaaaggccgttttaccatttctcgcgacaacagcaagaacacactgtacctgcagatgaactcactgcgtgccgaagatacggccgtgtattactgtgcgagatcttacgcttaccagtaccgtggcttggactactggggccagggaaccttggtcaccgtctcg

SEQ ID NO: 82 - DNA, 324 bp, Artificial Sequence
  IgG1 Light Chain MSM R610
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaaaataggacgtctcctatcacgttcggccaagggaccaaggtggaaatcaaa

SEQ ID NO: 83 - protein, 450 aa, Artificial Sequence
  IgG1 Heavy Chain MSM R610
EVQLLESGGGLVQPGGSLRLSCAASGFTFTGSSMSWVRQAPGKGLEWVSLISSRGSYTYYADSVKGRFTISRDNSKNTLYLQMNSLRAEDTAVYYCARSYAYQYRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 215 aa, Artificial Sequence
  IgG1 Light Chain MSM R610
EIVLTQSPGTLSLSPGERATLSCRASQSVSSSYLAWYQQKPGQAPRLLIYGASSRATGIPDRFSGSGSGTDFTLTISRLEPEDFAVYYCQQNRTSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 12 aa, Artificial Sequence
  IgG1 CDR1 Heavy Chain MSM R610
FTFTGSSMSWVR

SEQ ID NO: 86 - protein, 16 aa, Artificial Sequence
  IgG1 CDR2 Heavy Chain MSM R610
VSLISSRGSYTYYADS

SEQ ID NO: 87 - protein, 17 aa, Artificial Sequence
  IgG1 CDR3 Heavy Chain MSM R610
CARSYAYQYRGLDYWGQ

SEQ ID NO: 88 - protein, 12 aa, Artificial Sequence
  IgG1 CDR1 LightChain MSM R610
RASQSVSSSYLA

SEQ ID NO: 89 - protein, 7 aa, Artificial Sequence
  IgG1 CDR2 Light Chain MSM R610
GASSRAT

SEQ ID NO: 90 - protein, 13 aa, Artificial Sequence
  IgG1 CDR3 Light Chain MSM R610
CQQNRTSPITFGQ

SEQ ID NO: 91 - DNA, 360 bp, Artificial Sequence
  IgG1 Heavy Chain MSM R612
gaagttcaactgctggggtccggtggtggtctggtacagccgggtggttctctgcgtctgagttgcgcggccagtggctttaccttcactacctattatatgcattgggtgcgtcaggctccgggcaaaggtctggaatgggttagccttatttcgccgaatgcgggcaatacctactatgcggatagcgtgaaaggccgttttaccatttctcgcgacaacagcaagaacacgctgtacctgcagatgaactcactgcgtgccgaagatacggccgtgtattactgtgcgagaggccggtcgagggcggggacttccggcatggactactggggccagggaaccttggtcaccgtctcg

SEQ ID NO: 92 - DNA, 324 bp, Artificial Sequence
  IgG1 Light Chain MSM R612
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaagctgcttcttctcctatcacgttcggccaagggaccaaggtggaaatcaaa

SEQ ID NO: 93 - protein, 451 aa, Artificial Sequence
  IgG1 Heavy Chain MSM R612
EVQLLGSGGGLVQPGGSLRLSCAASGFTFTTYYMHWVRQAPGKGLEWVSLISPNAGNTYYADSVKGRFTISRDNSKNTLYLQMNSLRAEDTAVYYCARGRSRAGTSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 215 aa, Artificial Sequence
  IgG1 Light Chain MSM R612
EIVLTQSPGTLSLSPGERATLSCRASQSVSSSYLAWYQQKPGQAPRLLIYGASSRATGIPDRFSGSGSGTDFTLTISRLEPEDFAVYYCQQAASSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 12 aa, Artificial Sequence
  IgG1 CDR1 Heavy Chain MSM R612
FTFTTYYMHWVR

SEQ ID NO: 96 - protein, 16 aa, Artificial Sequence
  IgG1 CDR2 Heavy Chain MSM R612
VSLISPNAGNTYYADS

SEQ ID NO: 97 - protein, 18 aa, Artificial Sequence
  IgG1 CDR3 Heavy Chain AA MSM R612
CARGRSRAGTSGMDYWGQ

SEQ ID NO: 98 - protein, 12 aa, Artificial Sequence
  IgG1 CDR1 Light Chain MSM R612
RASQSVSSSYLA

SEQ ID NO: 99 - protein, 7 aa, Artificial Sequence
  IgG1 CDR2 Light Chain MSM R612
GASSRAT

SEQ ID NO: 100 - protein, 13 aa, Artificial Sequence
  IgG1 CDR3 Light Chain MSM R612
CQQAASSPITFGQ

SEQ ID NO: 101 - DNA, 351 bp, Artificial Sequence
  IgG1 Heavy Chain MSM R614
gaagtgcaactgctggagtccggtggtggtctggtacagccgggtggttctctgcgtctgagttgcccggccagtggctttaccttcactagatctgctatgagctgggtgcgtcaggctccgggcaaaggtctggaatgggttagctatattaattcgtctggtggcggtacctactatgcggatagcgtgaaaggccgttttaccatttctcgcgacaacagcaagaacacgctgtacctgcagatgaactcactgcgtgccgaagatacggccgtgtattactgtgcgagagcgaggttcatctgggggttcgactactggggccagggaaccctggtcaccgtctcg

SEQ ID NO: 102 - DNA, 324 bp, Artificial Sequence
  IgG1 Light Chain MSM R614
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatggtattgggctcctgtcacgttcggccaagggaccaaggtggaaatcaaa

SEQ ID NO: 103 - protein, 448 aa, Artificial Sequence
  IgG1 Heavy Chain MSM R614
EVQLLESGGGLVQPGGSLRLSCPASGFTFTRSAMSWVRQAPGKGLEWVSYINSSGGGTYYADSVKGRFTISRDNSKNTLYLQMNSLRAEDTAVYYCARARFIWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 215 aa, Artificial Sequence
  IgG LC AA MSM R614
EIVLTQSPGTLSLSPGERATLSCRASQSVSSSYLAWYQQKPGQAPRLLIYGASSRATGIPDRFSGSGSGTDFTLTISRLEPEDFAVYYCQQWYWAPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 12 aa, Artificial Sequence
  IgG1 CDR1 Heavy Chain MSM R614
FTFTRSAMSWVR

SEQ ID NO: 106 - protein, 16 aa, Artificial Sequence
  IgG1 CDR2 Heavy Chain MSM R614
VSYINSSGGGTYYADS

SEQ ID NO: 107 - protein, 15 aa, Artificial Sequence
  IgG1 CDR3 Heavy Chain MSM R614
CARARFIWGFDYWGQ

SEQ ID NO: 108 - protein, 12 aa, Artificial Sequence
  IgG1 CDR1 Light Chain MSM R614
RASQSVSSSYLA

SEQ ID NO: 109 - protein, 7 aa, Artificial Sequence
  IgG1 CDR2 Light Chain MSM R614
GASSRAT

SEQ ID NO: 110 - protein, 13 aa, Artificial Sequence
  IgG1 CDR3 Light Chain MSM R614
CQQWYWAPVTFGQ

SEQ ID NO: 111 - DNA, 369 bp, Artificial Sequence
  IgG1 Heavy Chain MSM R615
gaagttcaactgctggagtccggtggtggtctggtacagccgggtggttctctgcgtctgagttgcgcggccagtggctttaccttcagtagctattatatgcattgggtgcgtcagactccgggcaaaggtctggaatgggttagcattattagtccgtggagtagcagtacctactatgcggatagcgtgaaaggccgttttaccatttctcgcgacaacagcaagaacacgctgtacctgcagatgaactcactgcgtgccgaagatacggccgtgtattactgtgcgagatcttcttactcttggggtgaatcttacaaacgtgccatcgactactggggccagggaaccttggtcaccgtctcg

SEQ ID NO: 112 - DNA, 324 bp, Artificial Sequence
  IgG1 Light Chain MSM R615
gaaattgtgctgacccagtctccgggcacgttatctctgagccatggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatcttattattctcctgtcacgttcggccaagggaccaaggtggaaatcaaa

SEQ ID NO: 113 - protein, 454 aa, Artificial Sequence
  IgG1 Heavy Chain MSM R615
EVQLLESGGGLVQPGGSLRLSCAASGFTFSSYYMHWVRQTPGKGLEWVSIISPWSSSTYYADSVKGRFTISRDNSKNTLYLQMNSLRAEDTAVYYCARSSYSWGESYKRAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 215 aa, Artificial Sequence
  IgG1 Light Chain MSM R615
EIVLTQSPGTLSLSHGERATLSCRASQSVSSSYLAWYQQKPGQAPRLLIYGASSRATGIPDRFSGSGSGTDFTLTISRLEPEDFAVYYCQQSYYSPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 12 aa, Artificial Sequence
  IgG1 CDR1 Heavy Chain MSM R615
FTFSSYYMHWVR

SEQ ID NO: 116 - protein, 16 aa, Artificial Sequence
  IgG1 CDR2 Heavy Chain MSM R615
VSIISPWSSSTYYADS

SEQ ID NO: 117 - protein, 21 aa, Artificial Sequence
  IgG1 CDR3 Hevy Chain MSM R615
CARSSYSWGESYKRAIDYWGQ

SEQ ID NO: 118 - protein, 12 aa, Artificial Sequence
  IgG1 CDR1 Light Chain MSM R615
RASQSVSSSYLA

SEQ ID NO: 119 - protein, 7 aa, Artificial Sequence
  IgG1 CDR2 Light Chain MSM R615
GASSRAT

SEQ ID NO: 120 - protein, 13 aa, Artificial Sequence
  IgG1 CDR3 Light Chain MSM R615
CQQSYYSPVTFGQ

SEQ ID NO: 121 - DNA, 357 bp, Artificial Sequence
  IgG1 Heavy Chain MSM R617
gaagttcaactgctggagtccggtggtggtctggtacagccgggtggttctctgcgtctgagttgcgcggccagtggctttaccttcagtggctatgctatgcattgggtgcgtcaggctccgggcaaaggtctggaatgggttagctctattggttcgcgtaggggctttacctcttatgcggatagcgtgaaaggccgttttaccatttctcgcgacaacagcaagaacacgctgtacctgcagatgaactcactgcgtgccgaagatacggccgtgtattactgtgcgagatcttacgcttaccagtaccgtggcttggactactggggccagggaaccttggtcaccgtctcg

SEQ ID NO: 122 - DNA, 318 bp, Artificial Sequence
  IgG1 Light Chain MSM R617
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaaaggtcgtctgtcacgttcggccaagggaccaaggtggaaatcaaa

SEQ ID NO: 123 - protein, 450 aa, Artificial Sequence
  IgG1 Heavy Chain MSM R617
EVQLLESGGGLVQPGGSLRLSCAASGFTFSGYAMHWVRQAPGKGLEWVSSIGSRRGFTSYADSVKGRFTISRDNSKNTLYLQMNSLRAEDTAVYYCARSYAYQYRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 213 aa, Artificial Sequence
  IgG1 Light Chain MSM R617
EIVLTQSPGTLSLSPGERATLSCRASQSVSSSYLAWYQQKPGQAPRLLIYGASSRATGIPDRFSGSGSGTDFTLTISRLEPEDFAVYYCQQRSSVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 12 aa, Artificial Sequence
  IgG1 CDR1 Heavy Chain MSM R617
FTFSGYAMHWVR

SEQ ID NO: 126 - protein, 16 aa, Artificial Sequence
  IgG1 CDR2 Heavy Chain MSM R617
VSSIGSRRGFTSYADS

SEQ ID NO: 127 - protein, 17 aa, Artificial Sequence
  IgG1 CDR3 Heavy Chain MSM R617
CARSYAYQYRGLDYWGQ

SEQ ID NO: 128 - protein, 12 aa, Artificial Sequence
  IgG1 CDR1 Light Chain MSM R617
RASQSVSSSYLA

SEQ ID NO: 129 - protein, 7 aa, Artificial Sequence
  IgG1 CDR2 Light Chain MSM R617
GASSRAT

SEQ ID NO: 130 - protein, 11 aa, Artificial Sequence
  IgG1 CDR3 Light Chain MSM R617
CQQRSSVTFGQ

SEQ ID NO: 131 - DNA, 366 bp, Artificial Sequence
  IgG1 Heavy Chain MSM R619
gaagttcaactgctggagtccggtggtggtctggtacagccgggtggttctctgcgtctgagttgcgcggccagtggctttaccttcagtaactattatatccattgggtgcgtcaggctccgggcaaaggtctggaatgggttagcctgatttctccgggtggtagcactacctactatgcggatagcgtgaaaggccgttttaccatttctcgcgacaacagcaagaacacgctgtacctgcagatgaactcactgcgtgccgaagatacggccgtgtattactgtgcgagagggaggacttggtggtcgacttactattccggcattgactactggggccagggaaccttggtcaccgtctcg

SEQ ID NO: 132 - DNA, 324 bp, Artificial Sequence
  IgG1 Light Chain MSM R619
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatattcttcttctcctatcacgttcggccaagggaccaaggtggaaatcaaa

SEQ ID NO: 133 - protein, 453 aa, Artificial Sequence
  IgG1 Heavy Chain MSM R619
EVQLLESGGGLVQPGGSLRLSCAASGFTFSNYYIHWVRQAPGKGLEWVSLISPGGSTTYYADSVKGRFTISRDNSKNTLYLQMNSLRAEDTAVYYCARGRTWWSTYYSGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 215 aa, Artificial Sequence
  IgG1 Light Chain MSM R619
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 12 aa, Artificial Sequence
  IgG1 CDR1 Heavy Chain MSM R619
FTFSNYYIHWVR

SEQ ID NO: 136 - protein, 16 aa, Artificial Sequence
  IgG1 CDR2 Heavy Chain MSM R619
VSLISPGGSTTYYADS

SEQ ID NO: 137 - protein, 20 aa, Artificial Sequence
  IgG1 CDR3 Heavy Chain MSM R619
CARGRTWWSTYYSGIDYWGQ

SEQ ID NO: 138 - protein, 12 aa, Artificial Sequence
  IgG1 CDR1 Light Chain MSM R619
RASQSVSSSYLA

SEQ ID NO: 139 - protein, 7 aa, Artificial Sequence
  IgG1 CDR2 Light Chain MSM R619
GASSRAT

SEQ ID NO: 140 - protein, 13 aa, Artificial Sequence
  IgG1 CDR3 Light Chain MSM R619
CQQYSSSPITFGQ

SEQ ID NO: 141 - DNA, 363 bp, Artificial Sequence
  IgG1 Heavy Chain MSM R621
gaagttcaactgctggagtccggtggtggtctggtacagccgggtggttctctgcgtctgagttgtgcggccagtggctttaccttcactacctattatatgcattgggtgcgtcaggctccgggcaaaggtctggaatgggttagcggtattggtccgcgtgggagctggaccggttatgcggatagcgtgaaaggccgttttaccatttctcgcgacaacagcaagaacacgctgtacctgcagatgaactcactgcgtgccgaagatacggccgtgtattactgtgcgagaggcagggcgttggtgtcgaattattccggcttggactactggggccagggaaccttggtcaccgtctcg

SEQ ID NO: 142 - DNA, 324 bp, Artificial Sequence
  IgG1 Light Chain MSM R621
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatattattcttctcctgtcacgttcggccaagggaccaaggtggaaatcaaa

SEQ ID NO: 143 - protein, 452 aa, Artificial Sequence
  IgG1 Heavy Chain MSM R621
EVQLLESGGGLVQPGGSLRLSCAASGFTFTTYYMHWVRQAPGKGLEWVSGIGPRGSWTGYADSVKGRFTISRDNSKNTLYLQMNSLRAEDTAVYYCARGRALVSNYSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 215 aa, Artificial Sequence
  IgG1 Light Chain MSM R621
EIVLTQSPGTLSLSPGERATLSCRASQSVSSSYLAWYQQKPGQAPRLLIYGASSRATGIPDRFSGSGSGTDFTLTISRLEPEDFAVYYCQQYYSSPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 12 aa, Artificial Sequence
  IgG1 CDR1 Heavy Chain MSM R621
FTFTTYYMHWVR

SEQ ID NO: 146 - protein, 16 aa, Artificial Sequence
  IgG1 CDR2 Heavy Chain MSM R621
VSGIGPRGSWTGYADS

SEQ ID NO: 147 - protein, 19 aa, Artificial Sequence
  IgG1 CDR3 Heavy Chain MSM R621
CARGRALVSNYSGLDYWGQ

SEQ ID NO: 148 - protein, 12 aa, Artificial Sequence
  IgG1 CDR1 Light Chain MSM R621
RASQSVSSSYLA

SEQ ID NO: 149 - protein, 7 aa, Artificial Sequence
  IgG1 CDR2 Light Chain MSM R621
GASSRAT

SEQ ID NO: 150 - protein, 13 aa, Artificial Sequence
  IgG1 CDR3 Light Chain MSM R621
CQQYYSSPVTFGQ

SEQ ID NO: 151 - DNA, 357 bp, Artificial Sequence
  IgG1 Heavy Chain MSM R622
gaagttcaactgctggagtccggtggtggtctggtacagccgggtggttctctgcgtctgagttgcgcagccagtggctttaccttcagtaaaaactctatgagctgggtgcgtcaggctccgggcaaaggtctggaatgggttagccttattacttcgtattctggctatacctactatgcggatagcgtgaaaggccgttttaccatttctcgcgacaacagcaagaacacgctgtacctgcagatgaactcactgcgtgccgaagatacggccgtgtattactgtgcgagatcttacgcttaccagtaccgtggcttggactactggggccagggaaccttggtcaccgtctcg

SEQ ID NO: 152 - DNA, 324 bp, Artificial Sequence
  IgG1 Light Chain MSM R622
gaaattgtgctgacccagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaaaatgggaggagtcctgtcacgttcggccaagggaccaaggtggaaatcaaa

SEQ ID NO: 153 - protein, 485 aa, Artificial Sequence
  IgG1 Heavy Chain MSM R622
EVQLLESGGGLVQPGGSLRLSCAASGFTFSKNSMSWVRQAPGKGLEWVSLITSYSGYTYYADSVKGRFTISRDNSKNTLYLQMNSLRAEDTAVYYCARSYAYQYRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDKSRWQQGNVFSCSVMHEALHNHYTQKSLSLSPGK

SEQ ID NO: 154 - protein, 215 aa, Artificial Sequence
  IgG1 Light Chain MSM R622
EIVLTQSPGTLSLSPGERATLSCRASQSVSSSYLAWYQQKPGQAPRLLIYGASSRATGIPDRFSGSGSGTDFTLTISRLEPEDFAVYYCQQNGRSPVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 12 aa, Artificial Sequence
  IgG1 CDR1 Heavy Chain MSM R622
FTFSKNSMSWVR

SEQ ID NO: 156 - protein, 16 aa, Artificial Sequence
  IgG1 CDR2 Heavy Chain MSM R622
VSLITSYSGYTYYADS

SEQ ID NO: 157 - protein, 17 aa, Artificial Sequence
  IgG1 CDR3 Heavy Chain MSM R622
CARSYAYQYRGLDYWGQ

SEQ ID NO: 158 - protein, 12 aa, Artificial Sequence
  Human CDR1 LC AA MSM R622
RASQSVSSSYLA

SEQ ID NO: 159 - protein, 7 aa, Artificial Sequence
  IgG1 CDR2 Light Chain MSM R622
GASSRAT

SEQ ID NO: 160 - protein, 13 aa, Artificial Sequence
  IgG1 CDR3 Light Chain MSM R622
CQQNGRSPVTFGQ

SEQ ID NO: 161 - DNA, 366 bp, Artificial Sequence
  IgG1 Heavy Chain MSM R623
gaagttcaactgctggagtccggtggtggtctggtacagccgggtggttctctgcgtctgagttgtgcggccagtggctttaccttcagtaactattatatccattgggtgcgtcaggctccgggcaaaggtctggaatgggttagcacgattggttcgagtaatggcacgacctactatgcggatagcgtgaaaggccgttttaccatttctcgcgacaacagcaagaacacgctgtacctgcagatgaactcactgcgtgccgaagatacggccgtgtattactgtgcgagaggcagggcggtgaagctggcttactattccgggtttgactactggggccagggaaccttggtcaccgtctcg

SEQ ID NO: 162 - DNA, 324 bp, Artificial Sequence
  IgG1 Light Chain MSM R623
gaaattgtgctgacccagtctccgggcacgttatctctgagccctggtgagcgcgccaccctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatattcttattctcctatcacgttcggccaagggaccaaggtggaaatcaaa

SEQ ID NO: 163 - protein, 453 aa, Artificial Sequence
  IgG1 Heavy Chain MSM R623
EVQLLESGGGLVQPGGSLRLSCAASGFTFSNYYIHWVRQAPGKGLEWVSTIGSSNGTTYYADSVKGRFTISRDNSKNTLYLQMNSLRAEDTAVYYCARGRAVKLAYYSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 215 aa, Artificial Sequence
  IgG1 Light Chain MSM R623
EIVLTQSPGTLSLSPGERATLSCRASQSVSSSYLAWYQQKPGQAPRLLIYGASSRATGIPDRFSGSGSGTDFTLTISRLEPEDFAVYYCQQYSYSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 12 aa, Artificial Sequence
  IgG1 CDR1 Heavy Chain MSM R623
FTFSNYYIHWVR

SEQ ID NO: 166 - protein, 16 aa, Artificial Sequence
  IgG1 CDR2 Heavy Chain MSM R623
VSTIGSSNGTTYYADS

SEQ ID NO: 167 - protein, 20 aa, Artificial Sequence
  IgG1 CDR3 Heavy Chain MSM R623
CARGRAVKLAYYSGFDYWGQ

SEQ ID NO: 168 - protein, 12 aa, Artificial Sequence
  IgG1 CDR1 Light Chain MSM R623
RASQSVSSSYLA

SEQ ID NO: 169 - protein, 7 aa, Artificial Sequence
  IgG1 CDR2 Light Chain MSM R623
GASSRAT

SEQ ID NO: 170 - protein, 13 aa, Artificial Sequence
  IgG1 CDR3 Light Chain MSM R623
CQQYSYSPITFGQ

SEQ ID NO: 171 - DNA, 396 bp, Artificial Sequence
  IgG1 Heavy Chain MSM R624
gaagttcaactgctggagtccggtggtggtctggtacagccgggtggttctctgcgtctgagttgcgcggccagtggctttaccttcagtagctatgccatgagctgggtgcgtcaggcgccgggcaaaggtctggaatgggttagcgcgattagcggtagtggcggtagcacgtactatgcggatagcgtgaaaggccgttttaccatttctcgcgacaacagcaagaacacgctgtacctgcagatgaactcactgcgtgccgaagatacggccgtgtattactgtgcgagacagtcttggtggcgttctaaaatgatgtacgactctcatctgtcttactcttctgcttctggcttcgactactggggccagggaaccttggtcaccgtctcg

SEQ ID NO: 172 - DNA, 324 bp, Artificial Sequence
  IgG1 Light Chain MSM R624
gaaattgtgttgacgcagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatcttcttcttctcctatcacgttcggccaagggaccaaggtggaaatcaaa

SEQ ID NO: 173 - protein, 463 aa, Artificial Sequence
  IgG1 Heavy Chain MSM R624
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQSWWRSKMMYDSHLSYSSASGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 215 aa, Artificial Sequence
  IgG1 Light Chain MSM R624
EIVLTQSPGTLSLSPGERATLSCRASQSVSSSYLAWYQQKPGQAPRLLIYGASSRATGIPDRFSGSGSGTDFTLTISRLEPEDFAVYYCQQSSSSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 12 aa, Artificial Sequence
  IgG1 CDR1 Heavy Chain MSM R624
FTFSSYAMSWVR

SEQ ID NO: 176 - protein, 16 aa, Artificial Sequence
  IgG1 CDR2 Heavy Chain MSM R624
VSAISGSGGSTYYADS

SEQ ID NO: 177 - protein, 30 aa, Artificial Sequence
  IgG1 CDR3 Heavy Chain MSM R624
CARQSWWRSKMMYDSHLSYSSASGFDYWGQ

SEQ ID NO: 178 - protein, 12 aa, Artificial Sequence
  IgG1 CDR1 Light Chain MSM R624
RASQSVSSSYLA

SEQ ID NO: 179 - protein, 7 aa, Artificial Sequence
  IgG1 CDR2 Light Chain MSM R624
GASSRAT

SEQ ID NO: 180 - protein, 13 aa, Artificial Sequence
  IgG1 CDR3 Light Chain MSM R624
CQQSSSSPITFGQ

SEQ ID NO: 181 - DNA, 354 bp, Artificial Sequence
  IgG1 Heavy Chain MSM R625
gaagttcaactgctggagtccggtggtggtctggtacagccgggtggttctctgcgtctgagttgcgcggccagtggctttaccttcagtagctatgccatgagctgggtgcgtcaggcgccgggcaaaggtctggaatgggttagcgcgattagcggtagtggcggtagcacgtactatgcggatagcgtgaaaggccgttttaccatttctcgcgacaacagcaagaacacgctgtacctgcagatgaactcactgcgtgccgaagatacggccgtgtattactgtgcgagacagaacgtttggtacggtggcttggactactggggccagggaaccttggtcaccgtctcg

SEQ ID NO: 182 - DNA, 324 bp, Artificial Sequence
  IgG1 Light Chain MSM R625
gaaattgtgttgacgcagtctccgggcacgttatctctgagccctggtgagcgcgccactctgtcatgccgggcttctcaaagtgttagcagtagctacctggcgtggtatcagcaaaaaccgggccaggccccgcgtctgctgatttacggtgcatccagccgtgccaccggcattccagatcgtttttccggtagtggttctgggacggacttcactctgacaatctcacgcctggaaccggaggattttgcggtgtattactgccagcaatattcttatgctcctatcacgttcggccaagggaccaaggtggaaatcaaa

SEQ ID NO: 183 - protein, 449 aa, Artificial Sequence
  IgG1 Heavy Chain MSM R625
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQNVWYGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 215 aa, Artificial Sequence
  IgG1 Light Chain MSM R625
EIVLTQSPGTLSLSPGERATLSCRASQSVSSSYLAWYQQKPGQAPRLLIYGASSRATGIPDRFSGSGSGTDFTLTISRLEPEDFAVYYCQQYSYAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 12 aa, Artificial Sequence
  IgG1 CDR1 Heavy Chain MSM R625
FTFSSYAMSWVR

SEQ ID NO: 186 - protein, 16 aa, Artificial Sequence
  IgG1 CDR2 Heavy Chain MSM R625
VSAISGSGGSTYYADS

SEQ ID NO: 187 - protein, 14 aa, Artificial Sequence
  IgG1 CDR3 Heavy Chain MSM R625
CARQNVWYGGLDYW

SEQ ID NO: 188 - protein, 12 aa, Artificial Sequence
  IgG1 CDR1 Light Chain MSM R625
RASQSVSSSYLA

SEQ ID NO: 189 - protein, 7 aa, Artificial Sequence
  IgG1 CDR2 Light Chain MSM R625
GASSRAT

SEQ ID NO: 190 - protein, 13 aa, Artificial Sequence
  IgG1 CDR3 Light Chain MSM R625
CQQYSYAPITFGQ

SEQ ID NO: 191 - protein, 119 aa, Artificial Sequence
  IgG1 Heavy Chain MSM R626
EVQLLESGGGLVQPGGSLRLSCAASGFTFIYYYMSWVRQAPGKGLEWVSSISPYYGYTSYADSVKGRFTISRDNSKNTLYLQMNSLRAEDTAVYYCARRTWWLRGMDYWGQGTLVTVSS

SEQ ID NO: 192 - protein, 108 aa, Artificial Sequence
  Human LC AA MSM R626
EIVLTQSPGTLSLSPGERATLSCRASQSVSSSYLAWYQQKPGQAPRLLIYGASSRATGIPDRFSGSGSGTDFTLTISRLEPEDFAVYYCQQSSYYPITFGQGTKVEIK

SEQ ID NO: 193 - protein, 12 aa, Artificial Sequence
  IgG1 CDR1 Heavy Chain MSM R626
FTFIYYYMSWVR

SEQ ID NO: 194 - protein, 16 aa, Artificial Sequence
  IgG1 CDR2 Heavy Chain MSM R626
VSSISPYYGYTSYADS

SEQ ID NO: 195 - protein, 16 aa, Artificial Sequence
  IgG1 CDR3 Heavy Chain MSM R626
CARRTWWLRGMDYWGQ

SEQ ID NO: 196 - protein, 12 aa, Artificial Sequence
  IgG1 CDR1 Light Chain MSM R626
RASQSVSSSYLA

SEQ ID NO: 197 - protein, 7 aa, Artificial Sequence
  IgG1 CDR2 MSM R626
GASSRAT

SEQ ID NO: 198 - protein, 13 aa, Artificial Sequence
  IgG1 CDR3 Light Chain MSM R626
CQQSSYYPITFGQ

SEQ ID NO: 199 - protein, 122 aa, Artificial Sequence
  IgG1 Heavy Chain MSM R627
EVQLLESGGGLVQPGGSLRLSCAASGFTISSYMAWVRQAPGKGLEWVSSISPSSGSTYYADSVKGRFTISRDNSKNTLYLQMNSLRAEDTAVYYCARSMWSKQMHRYGLDYWGQGTLVTVSS

SEQ ID NO: 200 - protein, 108 aa, Artificial Sequence
  IgG1 Light Chain M627
EIVLTQSPGTLSLSPGERATLSCRASQSVSSSYLAWYQQKPGQAPRLLIYGASSRATGIPDRFSGSGSGTDFTLTISRLEPEDFAVYYCQQASSDPITFGQGTKVEIK

SEQ ID NO: 201 - protein, 11 aa, Artificial Sequence
  IgG1 CDR1 Heavy Chain MSM R627
FTISSYMAWVR

SEQ ID NO: 202 - protein, 16 aa, Artificial Sequence
  IgG1 CDR2 Heavy Chain MSM R627
VSSISPSSGSTYYADS

SEQ ID NO: 203 - protein, 20 aa, Artificial Sequence
  IgG1 CDR3 Heavy Chain MSM R627
CARSMWSKQMHRYGLDYWGQ

SEQ ID NO: 204 - protein, 12 aa, Artificial Sequence
  IgG1 CDR1 Light Chain MSM R627
RASQSVSSSYLA

SEQ ID NO: 205 - protein, 7 aa, Artificial Sequence
  IgG1 CDR2 Light Chain MSM R627
GASSRAT

SEQ ID NO: 206 - protein, 13 aa, Artificial Sequence
  IgG1 CDR3 Light Chain MSM R627
CQQASSDPITFGQ

SEQ ID NO: 207 - protein, 120 aa, Artificial Sequence
  IgG1 Heavy Chain MSM R628
EVQLLESGGGLVQPGGSLRLNCAASGFTFSSYAMSWVRQAPGKGLEWVSAISGSGGSTYYADSVKGRFTISRDNSKNTLYLQMNSLRAEDTAVYYCAKKGYYGGMAMDYWGQGTLVTVSS

SEQ ID NO: 208 - protein, 108 aa, Artificial Sequence
  IgG1 LIght Chain MSM R628
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPLTFGQGTKVEIK

SEQ ID NO: 209 - protein, 12 aa, Artificial Sequence
  IgG1 CDR1 Heavy Chain MSM R628
FTFSSYAMSWVR

SEQ ID NO: 210 - protein, 16 aa, Artificial Sequence
  IgG1 CDR2 Heavy Chain MSM R628
VSAISGSGGSTYYADS

SEQ ID NO: 211 - protein, 17 aa, Artificial Sequence
  IgG1 CDR3 Heavy Chain MSM R628
CAKKGYYGGMAMDYWGQ

SEQ ID NO: 212 - protein, 12 aa, Artificial Sequence
  IgG1 CDR1 Light Chain MSM R628
RASQSVSSSYLA

SEQ ID NO: 213 - protein, 7 aa, Artificial Sequence
  IgG1 CDR2 Light Chain MSM R628
GASSRAT

SEQ ID NO: 214 - protein, 13 aa, Artificial Sequence
  IgG1 CDR3 Light Chain MSM R628
CQQYSSSPLTFGQ

SEQ ID NO: 215 - protein, 120 aa, Artificial Sequence
  IgG1 Heavy Chain MSM R629
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYHHGMKFGMDYWGQGTLVTVSS

SEQ ID NO: 216 - protein, 108 aa, Artificial Sequence
  IgG1 Light Chain MSM R629
EIVLTQSPGTLSLSPGERATLSCRASQSVSSSYLAWYQQKPGQAPRLLIYGASSRATGIPDRFSGSGSGTDFTLTISRLEPEDFAVYYCQQSSSSPITFGQGTKVEIK

SEQ ID NO: 217 - protein, 12 aa, Artificial Sequence
  IgG1 CDR1 Heavy Chain MSM R629
FTFSSYAMSWVR

SEQ ID NO: 218 - protein, 16 aa, Artificial Sequence
  IgG1 CDR2 Heavy Chain MSM R629
VSAISGSGGSTYYADS

SEQ ID NO: 219 - protein, 17 aa, Artificial Sequence
  IgG1 CDR3 Heavy Chain MSM R629
CARYHHGMKFGMDYWGQ

SEQ ID NO: 220 - protein, 12 aa, Artificial Sequence
  IgG1 CDR1 Light Chain MSM R629
RASQSVSSSYLA

SEQ ID NO: 221 - protein, 7 aa, Artificial Sequence
  IgG1 CDR2 Light Chain MSM R629
GASSRAT

SEQ ID NO: 222 - protein, 13 aa, Artificial Sequence
  IgG1 CDR3 Light Chain MSM R629
CQQSSSSPITFGQ

SEQ ID NO: 223 - protein, 120 aa, Artificial Sequence
  IgG1 Heavy Chain MSM R630
EVQLLESGGGLVQPGGSLRLNCAASGFTFSSYAMSWVRQAPGKGLEWVSAISGSGGSTYYADSVKGRFTISRDNSKNTLYLQMNSLRAEDTAVYYCARKSYYSWPALDYWGQGTLVTVSS

SEQ ID NO: 224 - skipped

SEQ ID NO: 225 - protein, 12 aa, Artificial Sequence
  IgG1 CDR1 Heavy Chain MSM R630
FTFSSYAMSWVR

SEQ ID NO: 226 - protein, 12 aa, Artificial Sequence
  IgG1 CDR2 Heavy Chain MSM R630
FTFSSYAMSWVR

SEQ ID NO: 227 - protein, 17 aa, Artificial Sequence
  IgG1 CDR3 Heavy Chain MSM R630
CARKSYYSWPALDYWGQ

SEQ ID NO: 228 - protein, 12 aa, Artificial Sequence
  IgG1 CDR1 Light Chain MSM R630
RASQSVSSSYLA

SEQ ID NO: 229 - protein, 7 aa, Artificial Sequence
  IgG1 CDR2 Light Chain MSM R630
GASSRAT

SEQ ID NO: 230 - skipped

SEQ ID NO: 231 - protein, 123 aa, Artificial Sequence
  IgG1 Heavy Chain MSM R631
EVQLLESGGGLVQPGGSLRLSCAASGFTITAYAMAWVRQAPGKGLEWVSYISSDSGYTYYADIVKGRFTISRDNSKNTLYLQMNSLRAEDTAVYYCARSLYSRRSSSRGMDYWGQGTLVTVSS

SEQ ID NO: 232 - protein, 108 aa, Artificial Sequence
  IgG1 Light Chain MSM R631
EIVLTQSPGTLSLSPGERATLSCRASQSVSSSYLAWYQQKPGQAPRLLIYGASSRATGIPDRFSGSGSGTDFTLTISRLEPEDFAVYYCQQSYSAPVTFGQGTKVEIK

SEQ ID NO: 233 - protein, 12 aa, Artificial Sequence
  IgG1 CDR1 Heavy Chain MSM R631
FTITAYAMAWVR

SEQ ID NO: 234 - protein, 16 aa, Artificial Sequence
  IgG1 CDR2 Heavy Chain MSM R631
VSYISSDSGYTYYADI

SEQ ID NO: 235 - protein, 20 aa, Artificial Sequence
  IgG1 CDR3 Heavy Chain MSM R631
CARSLYSRRSSSRGMDYWGQ

SEQ ID NO: 236 - protein, 12 aa, Artificial Sequence
  IgG1 CDR1 Light Chain MSM R631
RASQSVSSSYLA

SEQ ID NO: 237 - protein, 7 aa, Artificial Sequence
  IgG1 CDR2 Light Chain MSM R631
GASSRAT

SEQ ID NO: 238 - protein, 13 aa, Artificial Sequence
  IgG1 CDR3 Light Chain MSM R631
CQQSYSAPVTFGQ

SEQ ID NO: 239 - protein, 119 aa, Artificial Sequence
  IgG1 Heavy Chain MSM R632
EVQLLESGGGLVQPGGSLRLSCAASGFTLTAYSISWVRQAPGKGLEWVSSIYSYSGSTYYADSVKGRFTISRDNSKNTLYLQMNSLRAEDTAVYYCARYYSYSYAMDYWGQGTLVTVSS

SEQ ID NO: 240 - protein, 108 aa, Artificial Sequence
  IgG1 LIght Chain MSM R632
EIVLTQSPGTLSLSPGERATLSCRASQSVSSSYLAWYQQKPGQAPRLLIYGASSRATDIPDRFSGSGSGTDFTLTISRLEPEDFAVYYCQQYSYSPVTFGQGTKVEIK

SEQ ID NO: 241 - protein, 12 aa, Artificial Sequence
  IgG1 CDR1 Heavy Chain MSM R632
FTLTAYSISWVR

SEQ ID NO: 242 - protein, 16 aa, Artificial Sequence
  IgG1 CDR2 Heavy Chain MSM R632
VSSIYSYSGSTYYADS

SEQ ID NO: 243 - protein, 16 aa, Artificial Sequence
  IgG1 CDR3 Heavy Chain MSM R632
CARYYSYSYAMDYWGQ

SEQ ID NO: 244 - protein, 12 aa, Artificial Sequence
  IgG1 CDR1 Light Chain MSM R632
RASQSVSSSYLA

SEQ ID NO: 245 - protein, 7 aa, Artificial Sequence
  IgG1 CDR2 Light Chain MSM R632
GASSRAT

SEQ ID NO: 246 - protein, 13 aa, Artificial Sequence
  IgG1 CDR3 Light Chain MSM R632
CQQYSYSPVTFGQ

SEQ ID NO: 247 - protein, 124 aa, Artificial Sequence
  IgG1 Heavy Chain MSM R633
EVQLLESGGGLVQPGGSLRLSCAASGFTFTSSYMAWVRQAPGKGLEWVSSIAPSSGSTYYADSVKGRFTISRDNSKNTLYLQMNSLRAEDTAVYYCARSRWEWYYHSSHGLDYWGQGTLVTVSS

SEQ ID NO: 248 - protein, 108 aa, Artificial Sequence
  IgG1 Light Chain MSM R633
EIVLTQSPGTLSLSPGERATLSCRASQSVSSSYLAWYQQKPGQAPRLLIYGASSRATDIPDRFSGSGSGTDFTLTISRLEPEDFAVYYCQQSYSDPITFGQGTKVEIK

SEQ ID NO: 249 - protein, 12 aa, Artificial Sequence
  IgG1 CDR1 Heavy Chain MSM R633
FTFTSSYMAWVR

SEQ ID NO: 250 - protein, 16 aa, Artificial Sequence
  IgG1 CDR2 Heavy Chain MSM R633
VSSIAPSSGSTYYADS

SEQ ID NO: 251 - protein, 21 aa, Artificial Sequence
  IgG1 CDR3 Heavy Chain MSM R633
CARSRWEWYYHSSHGLDYWGQ

SEQ ID NO: 252 - protein, 12 aa, Artificial Sequence
  IgG1 CDR1 Light Chain MSM R633
RASQSVSSSYLA

SEQ ID NO: 253 - protein, 7 aa, Artificial Sequence
  IgG1 CDR2 Light Chain MSM R633
GASSRAT

SEQ ID NO: 254 - protein, 13 aa, Artificial Sequence
  IgG1 CDR3 Light Chain MSM R633
CQQSYSDPITFGQ

SEQ ID NO: 255 - protein, 121 aa, Artificial Sequence
  IgG1 Heavy Chain MSM R634
EVQLLESGGGLVQPGGSLRLSCAASGFTFTTYYMSWVRQAPGKGPEWVSNIAAGGATDYADSVKGRFTISRDNSKNTLYLQMNSLRAEDTAVYYCARGPWGRYHPMGFDYWGQGTLVTVSS

SEQ ID NO: 256 - protein, 108 aa, Artificial Sequence
  IgG1 Light Chain MSM R634
EIVLTQSPGTLSLSPGERATLSCRASQSVSSSYLAWYQQKPGQAPRLLIYGASSRATDIPDRFSGSGSGTDFTLTISRLEPEDFAVYYCQQAYYSPVTFGQGTKVEIK

SEQ ID NO: 257 - protein, 12 aa, Artificial Sequence
  IgG1 CDR1 Heavy Chain MSM R634
FTFTTYYMSWVR

SEQ ID NO: 258 - protein, 15 aa, Artificial Sequence
  IgG1 CDR2 Heavy Chain MSM R634
VSNIAAGGATDYADS

SEQ ID NO: 259 - protein, 19 aa, Artificial Sequence
  IgG1 CDR3 Heavy Chain MSM R634
CARGPWGRYHPMGFDYWGQ

SEQ ID NO: 260 - protein, 12 aa, Artificial Sequence
  IgG1 CDR1 Light Chain MSM R634
RASQSVSSSYLA

SEQ ID NO: 261 - protein, 7 aa, Artificial Sequence
  IgG1 CDR2 Light Chain MSM R634
GASSRAT

SEQ ID NO: 262 - protein, 13 aa, Artificial Sequence
  IgG1 CDR3 Light Chain MSM R634
CQQAYYSPVTFGQ

SEQ ID NO: 263 - protein, 450 aa, Artificial Sequence
  IgG4 Heavy Chain MSM R605
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARNSMKGQRKQRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 264 - protein, 215 aa, Artificial Sequence; identical to SEQ ID NO: 34
  IgG4 Light Chain MSM R605

SEQ ID NO: 265 - protein, 449 aa, Artificial Sequence
  IgG4 Heavy Chain MSM R606
EVQLLESGGGLVQPGGSLRLSCAASGFTFSNYYIHWVRQAPGKGLEWVSIINPWSGRTYYADSVKGRFTISRDNSKNTLYLQMNSLRAEDTAVYYCARGRLRSLSYSGIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 266 - protein, 215 aa, Artificial Sequence; identical to SEQ ID NO: 44
  LC AA MSM R606 IgG4

SEQ ID NO: 267 - protein, 449 aa, Artificial Sequence
  IgG4 Heavy Chain MSM R608
QVQLVQSGAEVKKPGSSVKVSCKASGGTLTSYYISWVRQAPGQGLEWMGYISPSSGYTYYAQKFQGRVTITADESTSTAYMELSSLRSEDTAVYYCARRYRGRGYVYGLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 268 - protein, 215 aa, Artificial Sequence; identical to SEQ ID NO: 64
  IgG4 Light Chain MSM R608

SEQ ID NO: 269 - protein, 450 aa, Artificial Sequence
  IgG4 Heavy Chain MSM R623
EVQLLESGGGLVQPGGSLRLSCAASGFTFSNYYIHWVRQAPGKGLEWVSTIGSSNGTTYYADSVKGRFTISRDNSKNTLYLQMNSLRAEDTAVYYCARGRAVKLAYYSGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 270 - protein, 215 aa, Artificial Sequence; identical to SEQ ID NO: 164
  IgG4 Light Chain MSM R623

(19) United States
(12) Patent Application Publication
(10) Pub. No.: US 2015/0337037 A1
(43) Pub. Date: Nov. 26, 2015

(54) HUMAN MONOCLONAL ANTIBODIES AGAINST HUMAN CHEMOKINE RECEPTOR CCR6

(71) Applicant: MSM Protein Technologies, Woburn, MA (US)

(72) Inventors: Eldar Kim, Belmont, MA (US); Viktoriia S. Vasilyeva, Somerville, MA (US); Svetlana Abbasova, Moscow Region (RU); Ekaterina Ivanova, Boston, MA (US); Elena Ovchinnikova, Moscow Region (RU); Andre Ulitin, Moscow Region (RU); Roman Mikhaylov, Moscow Region (RU)

(73) Assignee: MSM Protein Technologies, Woburn, MA (US)

(21) Appl. No.: 14/404,717

(22) PCT Filed: Mar. 14, 2013
(86) PCT No.: PCT/US13/31692

§ 371 Date: Dec. 1, 2014

Related U.S. Application Data
(60) Provisional application No. 61/692,995, filed on Aug. 24, 2012.
(60) Provisional application No. 61/655,750, filed on Jun. 5, 2012.

(30) Foreign Application Priority Data
Mar. 13, 2013 (US) 2013/030865

(51) Int. Cl. C07K 16/28

(52) U.S. Cl. CPC C07K 16/28; C07K 2317/21; C07K 2316/96

(57) ABSTRACT

Aspects of this invention include fully human antibodies or fragments thereof that bind specifically to human CCR6 receptor. Such antibodies or fragments thereof can be used to treat disorders involving over function of the CCR6 receptor, including cancers, inflammatory diseases and fibrotic diseases. Other uses include detection of human CCR6 receptor in biological samples for diagnostic or evaluative purposes.

### PRIORITY CLAIM

This application claims priority to U.S. Provisional Patent Application No. 61/692,995, filed 24 Aug. 2012, titled: “Human Monoclonal Antibodies Against Human Chemokine Receptor CCR6,” Inventors: Eldar Kim et al. This application also claims priority to PCT International Patent Application No. PCT/US2013/30865, filed 13 Mar. 2013, which claims priority to U.S. Provisional Patent Application No. 61/655,750, filed 5 Jun. 2012. Each of the above applications is incorporated herein fully by reference as if separately so incorporated.

### FIELD OF THE INVENTION

This invention relates to antibodies against human G-protein coupled receptors (GPCRs). Particularly, this invention relates to fully human antibodies and fragments thereof directed against GPCRs, as well as conjugates of such antibodies and fragments thereof with toxins or radionuclides aimed at killing cells to which the conjugates bind. More particularly, this invention relates to fully human antibodies and fragments thereof directed against the human chemokine receptor CCR6, and to the conjugates of such antibodies.

### BACKGROUND

Chemokines are molecules having diverse function. They are extracellular molecules that can initiate and/or maintain numerous cell processes, including chemotaxis, cell growth and in some cases, tumor growth, homing of malignant cells and metastasis. Chemokines are also intimately involved in trafficking cells of immune system and are implicated in numerous autoimmune diseases, inflammation, and response to viral, bacterial and other infections. Chemokines can act by binding to, activating, or inhibiting receptors known as chemokine receptors. Chemokine receptors are in the class of G-protein coupled receptors (GPCRs) that are multispanning membrane proteins, in which the protein has one or more regions that span a cellular membrane.

### SUMMARY

We disclose fully human antibodies that can specifically bind to human chemokine receptor CCR6 on the surfaces of living cells. The CCR6 receptor, also known as CD196 (cluster of differentiation 196) binds chemokine CCL20. We disclose 28 different antibodies with different variable domains (most differ in CDR3, a few have the same CDR3 but different CDR1 and CDR2 of the heavy chain; CDR stands for complementarity determining region) sequences. These fully human antibodies can be used as therapeutics for the treatment of different types of cancer, inflammation, and other diseases. These fully human antibodies selectively bind to human CCR6, as well as to cynomolgus monkey CCR6, and include antibodies having antagonist (neutralizing) properties. These antibodies can be used in the IgG4 format (IgG stands for immunoglobulin G) that generally does not induce killing of a cell to which the antibodies bind in the organism, or other IgG format, such as the IgG1 format, IgG2, IgG3, IgG4, or in other immune globulin format.

The IgG1 format is an antibody subclass capable of inducing antibody-dependent cellular cytotoxicity (ADCC) and complement-dependent cytotoxicity (CDC) thus causing the death of a cell to which IgG1 is bound. These antibodies or antibodies in other immune globulin format or antibody fragments in the form of Fab, scFv or other form can also be conjugated with toxins or radionuclides aimed at killing cells to which the conjugates bind; the form of antibody-based drug known in the filed as antibody-drug conjugate (ADC), which stands for Antibody-Drug Conjugate. Cancers such as prostate cancer, squamous cell carcinoma of head and neck, colorectal cancer, hepatocellular carcinoma, various B-cell malignances, pancreatic adenocarcinoma, thyroid papillary carcinoma and other types of cancer that express chemokine receptor CCR6 or employ the CCR6 and/or its natural ligand(s), such as well-established chemokine CCL20 and possibly CCL18, for their maintenance, growth, or expansion cells of the body that express CCR6, such as cells of immune system or other cells, are therapeutic targets for the fully human anti-CCR6 antibodies and fragments thereof or ADC of this invention.

The antibodies of this invention can be used for treatment of various inflammatory conditions and diseases in which CCR6 is implicated, such as Rheumatoid Arthritis (RA), Inflammatory Bowel Disease (IBD), pulmonary diseases, such as Pulmonary Fibrosis, Asthma, Chronic Obstructive Pulmonary Disorder (COPD), Cystic Fibrosis, Allergy, and Respiratory Syncytial Virus Disease (RSV), and other inflammatory diseases, such as Psoriasis. Non-infectious uveitis and various other ocular diseases can also be treated with the fully human anti-CCR6 antibodies and fragments thereof or ADC of this invention.

### BRIEF DESCRIPTION OF THE FIGURES

This invention is described with reference to specific embodiments thereof. Other features and aspects of this invention can be appreciated with reference to the Figures, in which:

FIG. 1 depicts a graph of fluorescence of cells expressing CCR6 or other GPCRs, and labeled with commercial anti-respective GPCR antibodies conjugated with fluorescent dye phycoerythrin (PE).

FIG. 2 depicts a graph of fluorescence of cells expressing human CCR6, or its cynomolgus monkey or mouse orthologs, or other GPCRs, in the presence of human antibody IgG1 MSM-R601 of this invention, and labeled with anti-human antibody Fc-PE commercial conjugate. Columns 1-22 show results for the cells (1) R1610-human CXCR1; (2) Cf2th-human CXCR2; (3) R1610-human CXCR3; (4) CHO-human CXCR4; (5) CHO-human CXCR5; (6) R1610-human CXCR6; (7) CHO-human CXCR7; (8) CHO-human CCR3; (9) CHO-human CCR4; (10) CHO-human CCR5; (11) R1610-human CCR6; (12) CHO-human CCR7; (13) R1610-human CCR9; (14) CHO-human CCR10; (15) CHO host cell line; (16) R1610 host cell line; (17) Cf2Th host cell line; (18) 293HEK host cell line; (19) CHO-human CCR6; (20) Cf2Th-human CCR6; (21) CHO-cyno[molgus monkey] CCR6; and (22) CHO mouse CCR6.

FIG. 3 depicts a graph of fluorescence of the cells as in FIG. 2 expressing CCR6 or other GPCRs, in the presence of human antibody IgG MSM-R602 of this invention and labeled with anti-human antibody Fc-PE commercial conjugate.

FIG. 4 depicts a graph of fluorescence of the cells as in FIG. 2 expressing CCR6 or other GPCRs, in the presence of human antibody IgG1 MSM-R604 of this invention and labeled with anti-human antibody Fc-PE commercial conjugate.

FIG. 5 depicts a graph of fluorescence of the cells as in FIG. 2 expressing CCR6 or other GPCRs, in the presence of human antibody IgG1-MSM-R605of this invention and labeled with anti-human antibody Fc-PE commercial conjugate.

FIG. 6 depicts a graph of fluorescence of the cells as in FIG. 2 expressing CCR6 or other GPCRs, in the presence of human antibody IgG1 MSM-R606 of this invention and labeled with anti-human antibody Fc-PE commercial conjugate.

FIG. 7 depicts a graph of fluorescence of the cells as in FIG. 2 expressing CCR6 or other GPCRs, in the presence of human antibody IgG1 MSM-R607 of this invention and labeled with anti-human antibody Fc-PE commercial conjugate.

FIG. 8 depicts a graph of fluorescence of the cells as in FIG. 2 expressing CCR6 or other GPCRs, in the presence of human antibody IgG1 MSM-R608 of this invention and labeled with anti-human antibody Fc-PE commercial conjugate.

FIG. 9 depicts a graph of fluorescence of the cells as in FIG. 2 expressing CCR6 or other GPCRs, in the presence of human antibody IgG1 MSM-R609 of this invention and labeled with anti-human antibody-PE commercial conjugate.

FIG. 10 depicts a graph of fluorescence of the cells as in FIG. 2 expressing CCR6 or other GPCRs, in the presence of human antibody IgG1 MSM-R612 of this invention and labeled with anti-human antibody Fc-PE commercial conjugate;

FIG. 11 depicts a graph of fluorescence of the cells as in FIG. 2 expressing CCR6 or other GPCRs, in the presence of human antibody IgG1 MSM-R614 of this invention and labeled with anti-human antibody Fc-PE commercial conjugate.

FIG. 12 depicts a graph of fluorescence of the cells as in FIG. 2 expressing CCR6 or other GPCRs, in the presence of human antibody IgG1 MSM-R615 of this invention and labeled with anti-human antibody Fc-PE commercial conjugate.

FIG. 13 depicts a graph of fluorescence of the cells as in FIG. 2 expressing CCR6 or other GPCRs, in the presence of human antibody IgG1 MSM-R617 of this invention and labeled with anti-human antibody FC-PE commercial conjugate.

FIG. 14 depicts a graph of fluorescence of the cells as in FIG. 2 expressing CCR6 or other GPCRs, in the presence of human antibody IgG1 MSM-R619 of this invention and labeled with anti-human antibody Fc-PE commercial conjugate.

FIG. 15 depicts a graph of fluorescence of the cells as in FIG. 2 expressing CCR6 or other GPCRs, in the presence of human antibody IgG1 MSM-R621 of this invention and labeled with anti-human antibody Fc-PE commercial conjugate.

FIG. 16 depicts a graph of fluorescence of the cells as in FIG. 2 expressing CCR6 or other GPCRs, in the presence of human antibody IgG1 MSM-R622 of this invention and labeled with anti-human antibody Fc-PE commercial conjugate.

FIG. 17 depicts a graph of fluorescence of the cells as in FIG. 2 expressing CCR6 or other GPCRs, in the presence of human antibody IgG1 MSM-R623 of this invention and labeled with anti-human antibody Fc-PE commercial conjugate.

FIG. 18 depicts a graph of fluorescence of CHO cells expressing human CCR6, cynoCCR6, and CHO host cells in the presence of varying concentration of IgG1 antibodies of this invention MSM-R601 and labeled with a commercial anti-human Fc PE-conjugate.

FIG. 19 depicts a graph of fluorescence of CHO cells expressing human CCR6, cynoCCR6, and CHO host cells in the presence of varying concentration of IgG1 antibodies of this invention MSM-R602 and labeled with a commercial anti-human Fc PE-conjugate.

FIG. 20 depicts a graph of fluorescence of CHO cells expressing human CCR6, cynoCCR6, and CHO host cells in the presence of varying concentration of IgG1 antibodies of this invention MSM-R604 and labeled with a commercial anti-human Fc PE-conjugate.

FIG. 21 depicts a graph of fluorescence of CHO cells expressing human CCR6, cynoCCR6, mouse CCR6 and CHO host cells in the presence of varying concentration of IgG1 antibodies of this invention MSM-R605 and labeled with a commercial anti-human Fc PE-conjugate.

FIG. 22 depicts a graph of fluorescence of CHO cells expressing human CCR6, cynoCCR6, mouse CCR6 and CHO host cells in the presence of varying concentration of IgG1 antibodies of this invention MSM-R606 and labeled with a commercial anti-human Fc PE-conjugate.

FIG. 23 depicts a graph of fluorescence of CHO cells expressing human CCR6, cynoCCR6, mouse CCR6 and CHO host cells in the presence of varying concentration of IgG1 antibodies of this invention MSM-R607 and labeled with a commercial anti-human Fc PE-conjugate.

FIG. 24 depicts a graph of fluorescence of CHO cells expressing human CCR6, cynoCCR6, mouse CCR6 and CHO host cells in the presence of varying concentration of IgG1 antibodies of this invention MSM-R608 and labeled with a commercial anti-human Fc PE-conjugate.

FIG. 25 depicts a graph of fluorescence of CHO cells expressing human CCR6, cynoCCR6, mouse CCR6 and CHO host cells in the presence of varying concentration of IgG1 antibodies of this invention MSM-R609 and labeled with a commercial anti-human Fc PE-conjugate.

FIG. 26 depicts a graph of fluorescence of CHO cells expressing human CCR6, cynoCCR6, mouse CCR6 and CHO host cells in the presence of varying concentration of IgG1 antibodies of this invention MSM-R612 and labeled with a commercial anti-human Fc PE-conjugate.

FIG. 27 depicts a graph of fluorescence of R1610 cells expressing human CCR6, and CHO cells expressing cynoCCR6, and mouse CCR6 and R1610 and CHO host cells in the presence of varying concentration of IgG1 antibodies of this invention MSM-R614 and labeled with a commercial anti-human Fc PE-conjugate.

FIG. 28 depicts a graph of fluorescence of R1610 cells expressing human CCR6, and CHO cells expressing cynoCCR6, and mouse CCR6 and R1610 and CHO host cells in the presence of varying concentration of IgG1 antibodies of this invention MSM-R615 and labeled with a commercial anti-human Fc PE-conjugate.

FIG. 29 depicts a graph of fluorescence of CHO cells expressing human CCR6, cynoCCR6, mouse CCR6 and CHO host cells in the presence of varying concentration of IgG1 antibodies of this invention MSM-R629 and labeled with a commercial anti-human Fc PE-conjugate.

FIG. 30 depicts a graph of fluorescence of CHO cells expressing human CCR6, cynoCCR6 and CHO host cells in the presence of varying concentration of IgG1 antibodies of this invention MSM-R619 and labeled with a commercial anti-human Fc PE-conjugate.

FIG. 31 depicts a graph of fluorescence of CHO cells expressing human CCR6, cynoCCR6, mouse CCR6 and CHO host cells in the presence of varying concentration of IgG1 antibodies of this invention MSM-R621 and labeled with a commercial anti-human Fc PE-conjugate.

FIG. 32 depicts a graph of fluorescence of CHO cells expressing human CCR6, cynoCCR6, mouse CCR6 and CHO host cells in the presence of varying concentration of IgG1 antibodies of this invention MSM-R622 and labeled with a commercial anti-human Fc PE-conjugate.

FIG. 33 depicts a graph of fluorescence of CHO cells expressing human CCR6, cynoCCR6, mouse CCR6 and CHO host cells in the presence of varying concentration of IgG1 antibodies of this invention MSM-R623 and labeled with a commercial anti-human Fc PE-conjugate.

FIG. 34 depicts a graph of inhibition of the increase in the intracellular Ca concentration in response to addition of human CCL20 to Chem-1 cells expressing human CCR6 in the presence of inhibiting IgG1 antibodies MSM-R605 at varying concentration.

FIG. 35 depicts a graph of inhibition of the increase in the intracellular Ca concentration in response to addition of human CCL20 to Chem-1 cells expressing human CCR6 in the presence of inhibiting IgG1 antibodies MSM-R606 at varying concentration.

FIG. 36 depicts a graph of inhibition of the increase in the intracellular Ca concentration in response to addition of human CCL20 to Chem-1 cells expressing human CCR6 in the presence of inhibiting IgG1 antibodies MSM-R607 at varying concentration.

FIGS. 37A and 37B depict sequence alignments of antibodies of this invention. FIG. 37A depicts sequence alignment of heavy chain variable regions (VH) of antibodies of this invention. FIG. 37B depicts sequence alignment of light chain variable regions (VL) of antibodies of this invention.

FIGS. 38A-38C depict graphs of MFI of cells exposed to IgG antibodies (MSM R605) of this invention. FIG. 38A depicts human PBMCs, FIG. 38B depicts Cynomologus monkey PBMCx, and FIG. 38C depicts mouse splenocytes.

FIG. 39 depicts graphs of binding of an antibody against CCR6 (MSM R605) to human PBMCs, cynomologus monkey PBMCs, and mouse splenocytes.

FIG. 40 depicts a summary histogram of binding of anti-CCR6 antibodies to a variety of cells that express GPCRs.

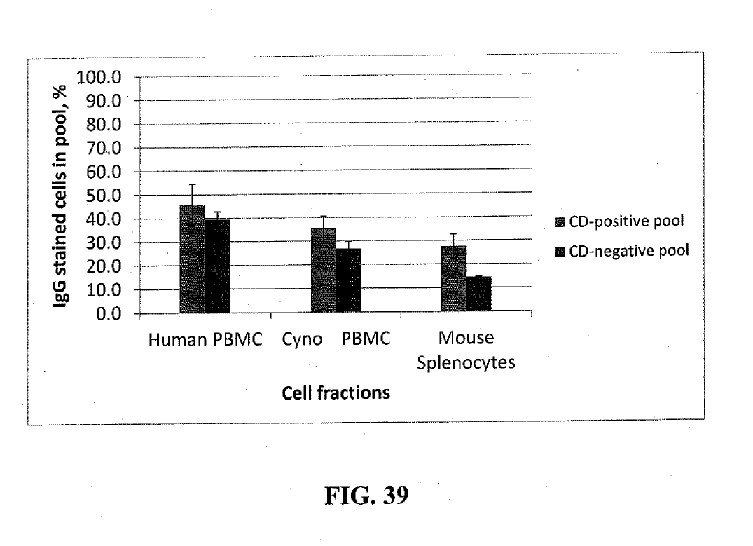

FIG. 41 depicts binding curves of an antibody of this invention (MSM 627 HC/JSJ R633LC) to human, cynomologus monkey, murine, and parental cells.

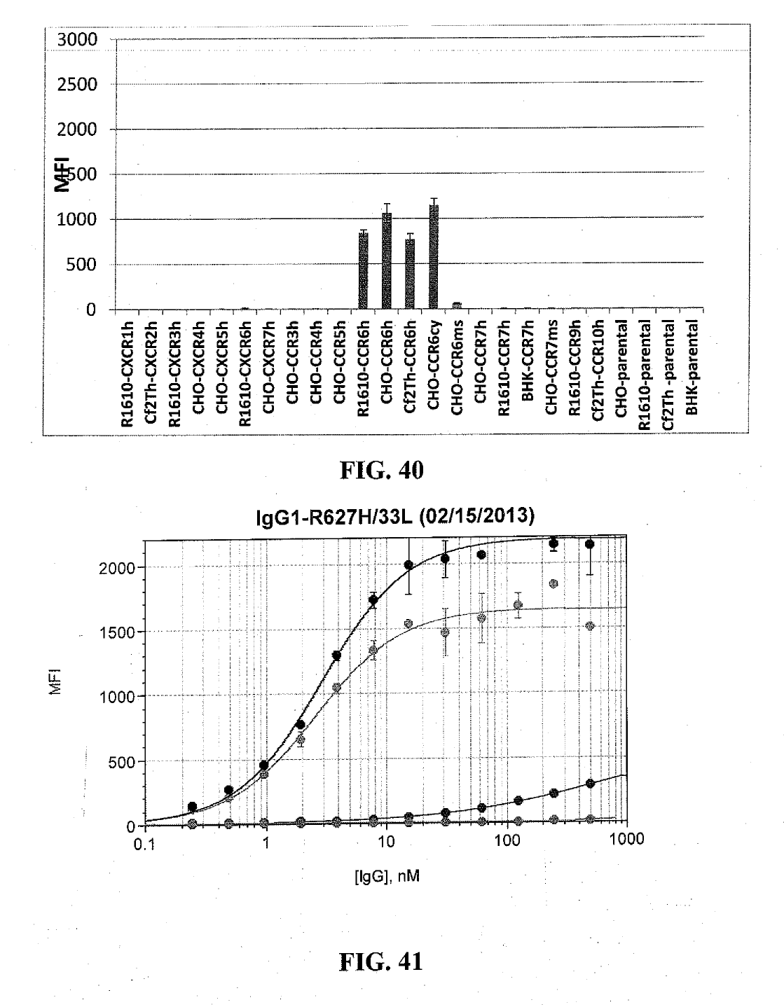

FIG. 42 depicts a photograph of a SDS-PAGE gel run under non-reducing (lanes 1-7) and reducing conditions (lanes 8-14) of the Antibodies of this invention in IgG4 format; respectively, MSM-R605 (lanes 2 and 8); R606 (3 and 9); R625 (4 and 10); R628 (5 and 11); R624 (6 and 12); also analyzed—a prior art IgG4 antibody, Rituximab (7 and 13); molecular weight markers were loaded onto the lanes 1 and 14; 3 microgram IgG4/lane.

### DETAILED DESCRIPTION

Aspects of this invention include fully human antibodies and fragments thereof directed against CCR6 and aimed at inhibiting CCL20 signaling via CCR6/CCL20 complex in cells expressing CCR6. The receptor is expressed on dendritic cells (DCs) and subsets of T- and B-cells and is implicated in lymphocyte activation and trafficking. The chemotaxis through CCR6/CCL20 is plays important role in homeostatic and inflammatory processes in mucosal surfaces, skin, brain, and eye. Accordingly, the disruption of the signaling via CCR6-CCL20 interaction was confirmed or strongly suggested to be beneficent for dumping aggravating inflammation in these tissues in a number of inflammatory diseases and conditions. As reported by a number of research groups and reviewed in Ito T, Carson W F 4th, Cavassani K A, Connett J M, Kunkel S L. CCR6 as a mediator of immunity in the lung and gut. Exp Cell Res. 2011 Mar. 10; 317(5):613-9 incorporated herein fully by reference, the involvement of CCR6 in mucosal immunity regulation is well documented. The involvement includes recruitment to the sites of inflammation of immature DCs and mature DCs, and professional antigen presenting cells (APCs). In the gut mucosal immunity, CCR6 is involved in homing of CD4(+) T (T-helper; Th) cells and DCs to the gut mucosal lymphoid tissue.

Several mechanisms might be involved, in particular, the CCR6-dependent engagement of pro-inflammatory cell types like Th-17 and others into the inflamed areas of the body. For mucosal surfaces, CCR6/CCL20-mediated response of goblet cells in the form of enhanced mucus production was recently suggested (Kim S, Lewis C, Nadel J A. CCL20/CCR6 feedback exaggerates epidermal growth factor receptor-dependent MUC5AC mucin production in human airway epithelial (NCI-H292) cells J. Immuno 2011 Mar. 15; 186(6):3392-400). Also, in inflammation and particularly autoimmune diseases, autocrine and paracrine mechanisms based in part on signaling via CCR6-CCL20 axes can be involved, in which inflammation is maintained via malicious cycle in which pro-inflammatory Th17 cells recruit via CCL20 immature DCs that upon maturation drive cell-mediated immunity fueling inflammation. In Rheumatoid Arthritis, as described by Hirota K, Yoshitomi H, Hashimoto M, Maeda S, Teradaira S, Sugimoto N, Yamaguchi T, Nomura T, Ito H, Nakamura T, Sakaguchi N, Sakaguchi S. Preferential recruitment of CCR6-expressing Th17 cells to inflamed joints via CCL20 in rheumatoid arthritis and its animal model. J Exp Med. 2007 Nov. 26; 204(12):2803-12, CCR6-expressing Th-17 cells also secrete the receptor's cognate ligand CCL20, that attract CCR6-expressing DC cells, and in addition, synoviocytes from arthritic joints of mice and humans also produce a large amount of CCL20. Mouse arthritis was inhibited by administration of a commercial mouse blocking anti-CCR6 monoclonal antibody. These results indicate that CCR6-CCL20 axis is intimately involved in autoimmune diseases, especially in autoimmune arthritis such as RA.

Regardless of whether the above described and/or other mechanisms are responsible for improvements of an inflammatory condition, a body of evidence was accumulated that supports the notion that fully human antibodies inhibiting CCR6 signaling of this invention can be used for treatment of a number of diseases, among which besides Rheumatoid Arthritis are pulmonary disorders such as COPD, Asthma, and RSV. It has become increasingly clear that various factors, such as external irritants or pathogens induce airway epithelial cells to produce CCL20, that in turn attracts from blood vessel immature dendritic cells (DCs) via chemotaxis mediated by CCR6 on their membrane, which upon uptake of the irritant or pathogen migrate to draining lymph node, where antigen is presented to T cells, which in turn proliferate and traffic back to the site of infection or irritation. For example, in COPD, as reported in the article titled “Accumulation of Dendritic Cells and Increased CCL20 Levels in the Airways of Patients with Chronic Obstructive Pulmonary Disease”; by Ingel K. Demedts et al., Am J Respir Crit Care Med V. 175. pp 998-1005, 2007, there is a significant increase in DC number in the epithelium and adventitia of small airways of patients with COPD compared with never-smokers and smokers without COPD. DC number in epithelium and adventitia increases along with disease severity. CCL20 mRNA expression in total lung and CCL20 protein levels in induced sputum are significantly higher in patients with COPD compared with never-smokers and smokers without COPD. DC isolated from human lung express CCR6 both at mRNA and at protein level.

In RSV (or hRSV; Human Respiratory Syncytial Virus Lung Disease), similarly role of CCL20/CCR6 axis in attracting DCs is well documented as a key element of immune response and often as an aggravating factor in the progression of the disease to a devastating level. The initial cause of the disease is the virus of hRSV. The virus infects multiple cell lines in vitro, whereas the epithelial cells of the respiratory tract are the major sites of virus replication in vivo. In adults and older children, RSV usually causes mild cold-like signs and symptoms, however, infants are most severely affected by RSV. They may markedly draw in their chest muscles and the skin between their ribs, indicating that they are having trouble breathing, and their breathing may be short, shallow and rapid. RSV is a common cause of bronchiolitis in infants (it is complicated by pneumonia in approximately 10% of cases). In the United States alone, RSV was estimated to be responsible for 70,000-126,000 pediatric hospitalizations yearly due to bronchiolitis or pneumonia, with an estimated 90-1900 deaths yearly (Shay et al., 1999; World Health Organization, 2005). Many of these infants progress to a chronic pulmonary condition that can contribute to development of airways disease.

Although RSV is considered as a pediatric disease it is also recognized as an important pathogen for the elderly and patients with chronic lung disease such as chronic obstructive pulmonary disease (COPD) and asthma and is associated with a mortality rate of 30-100% in immunosuppressed individuals. Moreover the role of RSV in acute exacerbations of COPD (AECOPD) causing prolonged episodes of illness has been recently appreciated. Recurrent infections with RSV are common and the disease is known to exist long after the virus has been cleared. The RSV disease pathology is clinically characterized by airway hyperreactivity (AHR), increased mucus production and inflammation. Evidences that the host response rather than the RSV virus is irresponsible for disease are:

(A) RSV infection is associated with a profound inflammatory response: RSV infection results in activation of NF-kB which leads to the induction of inflammatory chemokines and cytokines that contribute to inflammation by recruiting neutrophils, macrophages and lymphocytes to the airways (Miller et al., J Infect Dis 2004; 1898: 1419);

(B) RSV is not cytopathic: RSV can infected cells remain stably infected for several weeks. During this period large amounts of pro-inflammatory cytokines are produced by the cells, but in the absence of host effector cells, no cell injury is apparent. This is in stark contrast to the effect of influenza virus in the same system, which results in cell lysis within 48 h (Zhang at al.; J Virol 2002; 76(11): 5654-5666);

(C) Viral load correlates poorly with the disease severity. At the time of admission, viral load has usually reached its peak and falls at the same time as the illness becomes more severe. The anti-viral drug ribavirin is effective in reducing viral load, but does not affect disease outcome (Wright et al., J Infect Dis 2002; 185(8):1011-1018);

(D) Effect of formalin-inactivated vaccine: A formalin inactivated whole virus (FI-RSV) was used as a vaccine. On exposure to natural infection, vaccinees developed more severe disease than controls, resulting in 80% requiring hospital admission compared to 5% of controls and 2 children died (Kim et al., Am J Epidemiol 1969; 89(4): 422-434).

In an aspect of this invention therefore fully human antibodies of this invention blocking CCR6-CCL20 signaling can be used to reduce recruitment of DCs to dampen inflammation in RSV. In support of this, in an RSV model utilizing virus-challenged CCR6 knock-out mice a reduced pathophysiology and lower number of activated T-cells was observed in the lungs, viral clearance was early, and the beneficial effects of the absence of CCR6 were reversed supplementation of airways with appropriately obtained DCs (Kallal L E, Schaller M A, Lindell D M, Lira S A, Lukacs N W, CCL20/CCR6 blockade enhances immunity to RSV by impairing recruitment of DC. Eur J Immunol, 2010 April; 40(4):1042-52).

Furthermore, the fully human antibodies blocking CCR6-CCL20 signaling of this invention can also reduce mucus production, as was demonstrated to occur in the above mentioned CCR6-knock out model, which can be also partially due to inhibition of mucus-producing goblet cells that can express CCR6. The goblet cells inhibition by the fully human blocking antibodies of this invention can be helpful for treatment of Cystic Fibrosis, in which the body produces unusually thick, sticky mucus that clogs the lungs and leads to life-threatening lung infections, and in other disorders exacerbated by abnormal production of mucus.

As pertained to pulmonary disorders, certain embodiments of the invention include fragments of anti-CCR6 blocking antibodies that can be not fully human but have their frame modified to allow for formulation for inhalation of the antibody preparations that can be particular advantageous for reducing mucus production and inflammation in patients with COPD, asthma, cystic fibrosis, allergy, and others, as well as patients with pulmonary viral infection RSV in which the very viral infection is dramatically exacerbated by the body's own immune response. Further improvements are now available, due to the production of fully human anti-human CCR6 antibodies. Being fully human, these antibodies do not have the same potential problems associated with either non-human antibodies or chimeric antibodies.

Thus, other aspects of this invention include fully human antibodies and fragments thereof directed against CCR6 and conjugates of these antibodies or antibody fragments with toxins or radionuclides aimed at destroying cells to which such antibodies or ADC bind. CCR6 is involved in a number of malignances, and antibodies and fragments there that bind to CCR6 can result in decreased cancer growth and in suppression of homing of malignant cells and of metastases. In aspects of this invention, antibodies and fragments thereof are fully human, and in other aspects of this invention the fully human antibodies or their fragments are conjugated with toxins or radionuclides to directly kill cancer cells or cells involved in development of cancer or metastasis. The involvement of CCR6 in cancer is well documented for several types of cancer.

Squamous cell carcinoma of the head and neck (Wang, J., et al., Chemokine receptors 6 and 7 identify a metastatic expression pattern in squamous cell carcinoma of the head and neck. Adv Otorhinolaryngol, 2005. 62: p. 121-33). Thus, squamous cell carcinoma of the head and neck can be advantageously treated using antibodies of this invention.

Colorectal cancer (Ghadjar, P., et al., The chemokine CCL20 and its receptor CCR6 in human malignancy with focus on colorectal cancer. Int J Cancer, 2009. 125(4): p. 741-5; and, Brand, S., et al., Cell differentiation dependent expressed CCR6 mediates ERK-1/2, SAPK/JNK, and Akt signaling resulting in proliferation and migration of colorectal cancer cells. Journal of Cellular Biochemistry, 2006. 97(4): p. 709-723), as well as in colorectal cancer metastasis (Rubie, C., et al., Involvement of chemokine receptor CCR6 in colorectal cancer metastasis. Tumour Biol, 2006. 27(3): p. 166-74). These disorders also can be advantageously used to treat these types of cancer.

Hepatocellular carcinoma (Uchida, H., et al., Chemokine receptor CCR6 as a prognostic factor after hepatic resection for hepatocellular carcinoma. Journal of Gastroenterology and Hepatology, 2006. 21(1): p. 161-168). Additionally, B-Cell Lymphomas (Rehnm, A., at al.; Identification of a chemokine receptor profile characteristic for mediastinal large B-cell lymphoma. International Journal of Cancer, 2009. 125(10): p. 2367-2374), myeloma (Giullani, N., at al., CC-Chemokine Ligand 20/Macrophage Inflammatory Protein-3 (alpha) and CC-Chemokine Receptor 6 Are Overexpressed in Myeloma Microenvironment Related to Osteolytic Bone Lesions. Cancer Res, 2008. 68(16): p. 6840-6850) and prostate cancer (Ghadjar, P., at al., Chemokine receptor CCR6 expression level and aggressiveness of prostate cancer. Journal of Cancer Research and Clinical Oncology, 2008. 134(11): p. 1181-1189), as evidenced from the facts that CCR6 and its ligand CCL20 are both expressed in human samples derived from Prostate Cancer (PC) patients, PC derived cell line (PC3) also express CCR6 and CCL20, and in vivo data on suppression of tumor growth of PC3 cells additionally expressing CCL20 SC injected into a flank region of SCID/beige mice by SC injection of mouse Anti-human CCL20 mAb. (20 ug/mouse, 3 times/week), as described in WO2009156994 incorporated herein fully by reference. Thus, numerous different cancer types can be treated using antibodies of this invention.

In addition to cancer, fibrotic diseases also can be advantageously treated using fully human anti-human CCR6 antibodies. Such disorders include pulmonary fibrosis, inflammatory bowel disease, and psoriasis. In many such conditions, the concentration of chemokine CCL18 is increased, which has been linked to increase in the severity of the disease. CCL18 might be another natural ligand of CCR6. The chemokine can serve as early predictor of development of idiopathic pulmonary fibrosis and other types of fibrotic disorders. The administration of anti-CCR6 antibodies of this invention for treatment of these fibrotic disorders can be advantageously enhanced when the diagnostics based upon determination of CCL18 as a marked of disease progression is employed, which is one of the embodiments of the disclosure.

Even in the absence of antagonistic effects, important therapeutic effects of antibodies can arise from binding of antibodies to CCR6. In such cases, activation of ADCC and CDC mechanisms can result to the elimination of CCR6 expressing cells. Part of the mechanism is similar to the mechanism of how Rituxan™ is thought to eliminate CD20-expressing cells.

It should be appreciated that the above mechanisms are for purposes of illustration only, and are not considered to be the only mechanisms possible. The proper scope of this invention includes all possible mechanisms of action of the fully human antibodies of this invention.

In yet another embodiment of this invention, a combination of treatments aimed at (A) First, complete or partial elimination of CCR6-expressing cells by means of IgG1 fully human antibodies via CDC/ADCC or by means of ADC of the antibodies or antibody fragments; and, then (B) inhibiting signaling in residual and/or nascent CCR6-expressing cell populations via CCR6/CCL20 axis by means of blocking CCR6 with fully human IgG4 antibodies or antibody fragments of this invention. Such combination of this invention can be advantageous or even synergistic, as compare to employment of each individual treatment.

Besides above mentioned cancer and inflamatory conditions in can be in particular useful for treating autoimmune diseases such as Psoriasis (Mabuchi T, Chang. T W, Quinter S, Hwang S T. Chemokine receptors in the pathogenesis and therapy of psoriasis. J Dermatol Sci. 2012 January; 65(1):4-11), IBD (Kaser at al., Increased expression of CCL20 in human inflammatory bowel disease J Clin Immunol. 2004 January; 24(1):74-85), Autoimmune Uveitis (Chang-Rong Yu et al., Therapeutic Targeting of STAT3 (Signal Transducers and Activators of Transcription 3) Pathway Inhibits Experimental Autoimmune Uveitis, PLoS One. 2012; 7(1): e29742), Multiple Sclerosis (Meares G P, Ma X, Qin H, Benvaniste E N. Regulation of CCL20 expression in astrocytes by IL-6 and IL-17. Glia. 2012 May; 60(5):771-81), as well as other diseases.

Methods for Producing CCR6 Receptors

In certain embodiments of this invention, CCR6 receptors can be isolated from membranes of cells expressing the protein and used as an immunogen to produce CCR6-specific antibodies. In general, we used methods described in PCT International Patent Application No: PCT/US2007/003169, filed 5 Feb. 2007 (WO 2007/092457). This application is expressly incorporated herein fully by reference. The production of CCR6 in cell lines was confirmed by staining of the cells with a commercial anti-CCR6 antibody conjugated with a fluorescence protein PE and fluorescence flow cytometry, as described in details under Example 1 below.

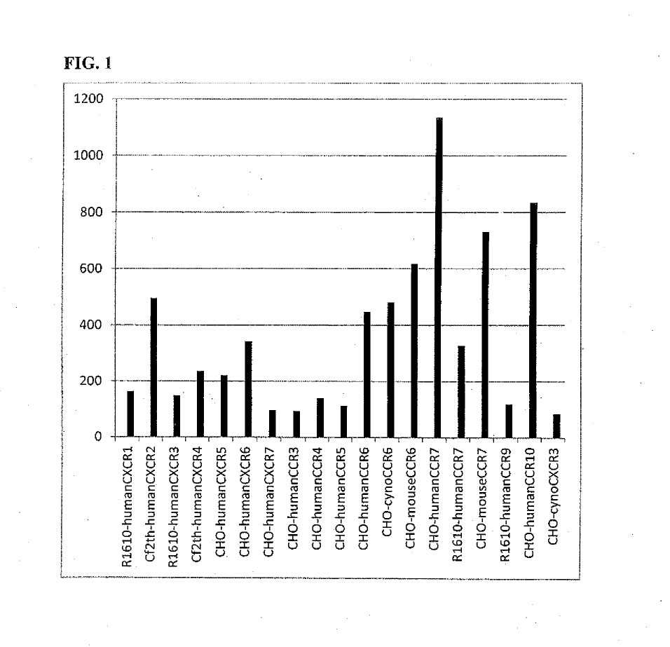

The data so obtained are provided in FIG. 1. The staining confirmed that all cell lines so produced displayed a high level of expression of their respective GPCR and thus were suitable for analysis of specificity of binding of anti-CCR6 antibodies of this invention.

### Applications of Human Antibodies Against Human CCR6

Fully human anti-CCR6 antibodies and/or fragments thereof and/or ADC generated by incorporation of toxins or radionuclides into the body of said antibodies as known to those skillful in the art (as reviewed, for example by Adair J R, Howard P W, Hartley J A, Williams D G, Chester K A. in “Antibody-drug conjugates—a perfect synergy.” Expert Opin Biol Ther. 2012 Jun. 1, which and references therein are incorporated herein fully by reference) can be used as therapeutic agents for different types of cancer and immunologic diseases where CCR6 plays a role. The types of cancer include: (1) Squamous cell carcinoma of the head and neck; (2) Colorectal cancer, as well as colorectal cancer metastasis; (3) Hepatocellular carcinoma; (4) B-Cell Lymphomas (5) Myeloma; (6) Prostate cancer; and others in which CCR6 can be implicated. Anti-CCR6 antibodies and fragments and/or ADC thereof may be used as a therapeutics for treatment of inflammatory diseases such as (1) Rheumatoid Arthritis; (2) Inflammatory Bowel Disease; (3) Psoriasis; (4) Multiple Sclerosis, (5) Asthma, (6) Allergies, and (7) Uveitis, and in a number of other pulmonary disease, such as (8) COPD, (9) RSV, (10) Cystic Fibrosis

However, it can be appreciated that other disorders involving CCR6 can be treated as well. Thus, the full scope of therapeutics involving antibodies and fragments thereof of this invention includes any disorder in which CCR6's actions are at least partially responsible for the disorder. As can be appreciated from Examples provided further herein, anti CCR6 antibodies of this invention in IgG1 format recognized well CCR6 on the surface of CCR6-expressing cells, including certain subsets of human PBMC, cynomolgus monkey PBMC and mouse splenocytes. The binding to CCR6 expressing cells was characterized by EC50 in nM range, which is a typical range for therapeutic antibodies. The binding can be used to induce elimination of CCR6-expressing cells in the body by means of CDC/ADCC, or alternatively the same goal can be achieved by using ADC. Several antibodies of this invention inhibit binding of CCL20, the natural ligand of CCR6, thereby demonstrating that therapeutic uses of fully human CCR6 antibodies can be a viable alternative to existing treatments for such disorders.

### Description of Antibodies

We have identified two functional classes of fully human anti-human CCR6 (“anti-CCR6”) antibodies:

1. Monoclonal Antibodies (MAbs) and fragments thereof which just bind to CCR6 but do not affect its natural ligand binding properties and signaling;

2. MAbs and fragments thereof which bind to CCR6 and inhibit the binding of natural ligands CCL20 to CCR6 (antagonists). Therefore they are called neutralizing MABs.

Antibodies of this invention can be IgG1, IgG2, IgG3, or IgG4 format, or other immune globulin formats. For some applications, it can be desirable to use anti-CCR6 antibodies of the IgG1 class. In contrast, IgG3 antibodies are strong activators of complement, IgG1 are also high, IgG2 antibodies are less able to activate complement, and IgG4 antibodies may activate complement only weakly.

In addition to full-length antibodies, portions of antibodies of this invention can also be used to target and/or bind to CCR6. For example, as shown in the Examples below, scFv or Fab fragments having CDRs of a respective full antibody, with typically CDR3 Heavy Chain (HC) regions determining their binding characteristics, can provide highly specific binding to target molecules.

All the antibodies were identified from phage display human antibody libraries presenting broad repertoire (up to 10¹²) of different human antibody Fab fragments (Fab libraries) using CCR6 Target Presentation Material of this invention in the form of CCR6-Golik™.

The search for antibodies or antibody fragments that bind external domains of membrane proteins remains a laborious and inefficient process. Two major approaches to the discovery of such ligands employ.

(1) Immunization with preparations of trans-membrane protein, such as cells, viral particles, or cellular membranes, or with peptide fragments of a trans-membrane protein, and discovery of antibodies that bind the target from obtained by immunization plurality of cells expressing various antibodies via formation of hybridoma cells or focused libraries obtained by PCR of B-cells from the serum of immunized animals; or

(2) A phage or other type of library containing a very large number of antibodies or antibody fragments linked to their respective genotype information (phage libraries, molecular libraries, mammalian cells libraries, bacterial libraries, yeast libraries, in which a member carries an antibody portion capable of binding to an antigen and genetic information on the variable portions of such antibody). Screening of such a library can result in the isolation from the library containing those library members that bind to a preparation of trans-membrane protein, such as cells, viral particles or cellular membranes, or to peptide fragments of a trans-membrane protein.

Both approaches are critically dependent upon the quality of membrane protein preparation, in the following referred to as Target Presentation Material (TPM): (a) When a trans-membrane protein in a TPM is present at a low concentration, immune response is poor, and isolation of antibodies that bind to the protein is problematic. (b) The presence of other protein and non-protein contaminants in the TPM, which is typically the case when whole cells, virus particles, or crude membrane preparations are used, makes background signal to be so strong that one is unable to identify the ligands in question. (c) The loss of native conformation of such a protein often renders both approaches at best inefficient and often futile.

The important requirements for quality (a) through (c) are poorly addressed by cell-based, viral particle-based, liposome-based, or membrane-based preparations of membrane proteins. Also, the use of fragments of membrane proteins has been proven inefficient because such fragments cannot maintain native conformation, and even when a ligand that binds such a peptide is discovered, such a ligand often is unable to exert a desirable change upon the function of a membrane protein.

Magnetic Proteoliposomes (MPLs) disclosed in the U.S. Pat. No. 6,761,902 titled ‘Proteoliposomes containing an integral membrane protein having one or more transmembrane domains’ by Joseph Sodroski and Tajib Mirzabekov, Jul. 13, 2004, and the US patent application 20010034432, A1, Oct. 25, 2001 titled ‘Proteoliposomes containing an integral membrane protein having one or more transmembrane domains’ by the same inventors, and the US patent application 20040109887, A1, Jun. 10, 2004 titled ‘Immunogenic proteoliposomes, and uses thereof’ by Wyatt, Richard T. et al, have been used as membrane protein preparations. Each of these publications and patent applications is incorporated herein fully by reference as if separately so incorporated.

MPLs allow one to purify a membrane protein in its native, functional conformation, and stabilize the protein in proper orientation and at high concentration on the surface of easy-to-handle magnetic beads. Membrane proteins in MPLs remain functionally intact due to carefully crafted membrane environment that encompasses certain added lipids. While MPLs have been proven effective in human antibody development using both transgenic mouse immunization and by selection of antibodies from phage display libraries, the need for carefully crafted and laborious selection of lipids and lipid reconstitution procedures makes this approach time consuming, expensive, and demanding highly sophisticated labor. In one embodiment of present invention, the methods of manufacturing and use of membrane-protein carrying particles that do not require laborious and expensive lipid-involving procedures are disclosed. The particles of this invention can carry CCR6 membrane protein molecules on their surface that are in proper orientation, highly concentrated and can be stabilized by certain detergents in native-like or native state (“naked particles”, or “Golik particles”). Using such Golik particles can dramatically reduce the time for selection of ligands from various libraries, such as chemical library, phage, aptamer, shpigelmer, nanobody, antibody fragment, scFv, minibody, anticalin or other protein scaffold library, call library, and any other library.

One of the embodiments of present invention discloses Golik™ particles that carry the CCR6 protein on their surface, and yet another embodiment of present invention discloses antibody-ligands that can bind the CCR6 protein exposed on the surface of these preparations and CCR6 on the surface of cells.

In contrast to the MPL preparation, no step of addition of lipid in order to reconstitute the lipid bilayer was employed. Presumably the lipid bilayer was not reconstituted completely, and only molecules of detergent—lipid mixture stabilize the protein structure. The Golik-CCR6 TPM of this invention was successfully used for selection of anti-CCR6 fully human antibodies of this invention from human antibody libraries.

In certain embodiments, the use of CCR6-Golik™ materials can be appreciated for other human GPCRs. For example, further description of manufacture and desirable physicochemical properties of GPCR-Golik™ materials are described in PCT/US2013/30865, filed Mar. 13, 2013. This patent application is herein incorporated fully by reference.

### Applications of Fully Human Anti-CCR6 Antibodies for Diagnostics and Research

Fully human antibodies and fragments thereof against CCR6 can be useful diagnostic and/or therapeutic agents in treatment of a variety of conditions in which CCR6 is overexpressed, or in which the ligand for CCR6 is over-expressed or released in pathological situations.

Fully human antibodies against human CCR6 of this invention that cross react with cynomolgus monkey CCR6 and/or mouse CCR6 can be useful in further development of drugs affecting CCR6 in human beings for treatment of a variety of diseases and conditions. Numerous mouse models have been and can be employed to demonstrate an efficacy of anti-CCR6 antibodies. Additionally, novel mouse and cynomolgus, marmoset, green monkey, and other monkey models relevant to human diseases are being developed for discovery of new drugs, and the CCR6 antibodies of this invention used these models are also predictive of effects seen in human beings. Cross reactivity of human antibodies of this invention with cyno CCR6 and mouse CCR6 can make the use of these models straightforward. Therefore, data obtained in these models using fully human antibodies are reasonably predictive of effects observed in human beings.

Thus, the antibodies of this invention can be useful for treatment of human diseases and conditions in which CCR6 and its natural ligand CCL20 are involved. For example, antibodies of this invention can be used to treat the following diseases.

Pulmonary Diseases

Immuno-Fibrotic Lung Diseases (IFLD) are poorly responsive to NSAID's, that are effective in Th1/Th2 inflammation and that inhibit iDC maturation. They are also poorly responsive to glucocorticoids (“GC”). However, GC's suppress IL-8 secretion and therefore neutrophil activation, GC's inhibit the production of interleukin (IL)-12, interferon (IFN)-gamma, IFN-alpha, and tumor necrosis factor (TNF)-alpha to down regulate Th1 response. Further, GC's inhibit iDC maturation and induce CCL20, IL-10 and IL-4 and therefore induce iDC, Th17, and CCR6⁺ neutrophil chemotaxis, activation and resistance to apoptosis as well as CCL18 expression. Immuno-fibrotic lung disease is poorly responsive to immuno-suppressive methotrexate that down regulates IL-12R and the CXCR3 receptors to inhibit the TH1 population. IFLDs are poorly responsive to calcineurin inhibitors that decrease Th1 responses by inhibiting IL-12 production and increase Th2 biased IL-10. IFLDs are poorly responsive to anti-TNF and anti-IL-1β therapies. (Modulate Th1 responses and have shown poor to mixed results in Th17 mediated diseases, e.g., IPF, asthma and COPD).

However, therapeutic intervention using anti-CCR6 antibodies of this invention is based on the following.

1. The “checkpoint” CCR6 chemokine controls, the Th17 pathway and systemic-to-local cell Th17 cell chemotaxis that are dysregulated in Immuno-fibrotic lung diseases including
Respiratory Syncytial Virus (“RSV”) Induced fibrosis.
Idiopathic Pulmonary Fibrosis (“IPF”)
Chronic Obstructive Pulmonary Disease (“COPD”)
Forms of severe Asthma
Fibrotic Sequelae of Acute Lung Injury/Adult Respiratory Distress Syndrome
There are no disease modifying drugs for these immuno-fibrotic pulmonary diseases.
The fully human, antibody drugs (in Mab or scFv formats) that can either block the CCR6 GPCR or that deplete CCR6+ cells.

Immunological Diseases

Cell mediated innate and adaptive Th17 dysregulation drive pulmonary Immuno-fibrosis, not humoral auto-antibodies. Excess Th17 responsive cell populations, including epithelial, dendritic, neutrophil, macrophage and fibrocytes drive local cellular Th17 mediated auto-immunity and fibrosis. Th17 cells express the CCR6⁺ cell surface marker. CCR6 mediates systemic Th17 cell trafficking to local sites of immuno-fibrosis and the Th17 cascade at sites of immuno-fibrosis. CCR6 blockade reduces Th17 cell migration to sites of immuno-fibrosis and reduces Th17 cell mediated immune cell activation, signaling and survival at sites of immuno-fibrosis. CCR6 is the “check point” chemokine receptor for iDC and Th17 cell chemotaxis and CCR6⁺ cell activation as well as for CCR6⁺ Akt pathway resistance to apoptosis (confirmed in CCR6 neutrophils).

Therefore, CCR6 blockade and/or CCR6 cell depletion can arrest excess iDC populations in immuno-fibrotic loci, reduce secretion of MUC-1 and MUC-5, reduce iDC IL-23 and CCL18 signaling in the lung, reduce IL-6, CCL18, IL-8 and Th17 activation of epithelial cells, neutrophils and macrophages, reduce activation and expansion of Th17 epithelial cells Th17 Fibrocyte and other CCR6+ cells, initiated by iDC's and inhibit down-stream cascades of CCL20/CCR6, IL-6, IL-8, IL-17, IL-23, CCL17, CCL18, CCL19 and CCL22 and mediated chemotaxis, activation and expansion of innate/adaptive immune cell and fibrocyte populations in autoimmune fibrotic lung diseases initiated by iDC's and other CCR6+ cells. It is known that systemic IL-17 cytokine blockade alone does not arrest excess immune cell chemotaxis and accumulation at sites of inflammation nor local fibrosis induced by invasive CCR6 Th17 cells and apoptosis resistant fibroblasts. Therefore anti-CCR6 antibodies of this invention can have an important therapeutic use in treating immunological disorders associate with either CCR6 over-expression or over-activation of CCR6 by ligands.

CCR6 KO/Blockade Proof of Concept Studies Suggest Efficacy in Immuno-Fibrotic Lung Diseases

Therapeutic uses of antibodies of this invention is supported by the findings that CCR6 blockade has shown anti-fibrosis efficacy in an RSV model of lung viral infection and immuno-fibrosis. Cigarette Smoke induced Pulmonary inflammation and emphysema are attenuated in CCR6 KO mice. Cockroach induced asthmatic responses are attenuated in CCR6 KO mice CCR6 blockade has shown anti-inflammatory efficacy in a model of Rheumatoid Arthritis. SNP's in the promoter region of CCR6 show high association of increased CCR6 expression with RA and other Th17 mediated autoimmune diseases, and CCR6 is required for induction of IL-23 psoriasis-like mouse model.

Respiratory Syncytial Virus

Respiratory Syncytial Virus (RSV) is a serious pulmonary disease that affects thousands of individuals. In pediatric patients, it is known that:

1. 70,000-126,000 pediatric hospitalizations for RSV induced bronchiolitis or pneumonia per year in the US

2. Many infants progress to chronic pulmonary conditions contribute to development of airways disease.

3. An unknown percent of pediatric patients require mechanical ventilation

4. 90-1900 RSV induced deaths yearly

5. Reduced Mechanical ventilation is a validated clinical outcome benefit.

6. US RSV healthcare costs in children <5>$US1 B;

7. >30% due to hospitalizations with mechanical ventilation.

In adults, it is know that:

1. RSV is an important pathogen for the elderly and patients with IPF, COPD and asthma

2. RSV causes acute exacerbations of IPF, COPD and asthma leading to prolonged episodes of illness and mechanical ventilation.

3. Recurrent infections with RSV are common and the immuno-fibrotic disease continues long after the virus has been cleared.

4. RSV is associated with a mortality rate of 30-100% in immuno-suppressed individuals.

Treatment of RSV Using Antibodies Against CCR6

There are several possible mechanisms of action of anti-CCR6 antibodies to treat RSV. Some mechanisms are based on the following:

1. Th17 activation suppresses Th1 mediated antiviral clearance and activates cellular immuno-fibrosis in RSV.

2. CCR6 blockade induces more rapid clearance of RSV by inducing a more robust Th1 response and blocking Th17 activation.

3. CCR6 blockade inhibit Th17 cellular auto-activation and immuno-fibrosis.

4. CCR6 will reduce acute mucus and inflammation leading to improved lung function.

5. Faster viral clearance, reduced inflammation and mucus secretion and reduced immuno-fibrosis in RSV infection will result in (i) better lung function, (i) fewer patients requiring ventilation, (iii) reduced patient mechanical ventilation days and (iv) reduced overall morbidity and mortality caused by RSV infection.

Idiopathic Pulmonary Fibrosis

Idiopathic pulmonary fibrosis (IPF) is a very serious set of disorders. By its name, the pathogenesis of IPF is poorly identified. IPF affects 140,000 patients, and is and drugs to treat IPF are considered orphan drugs. There is no approved standard of care or efficacious off label regimen. Biologic and cellular markers confirm Auto-immune dysregulation in IPF. These factors can lead to

1. Chronic; accelerating inflammation and mucus secretion,

2. Progressive auto-immune acceleration mediated by excess populations of apoptosis resistant: iDC's, CCR6⁺ neutrophils, alternatively activated macrophages, Th17 cells and IL-17 responsive fibrocytes,

3. Progressive remodeling of airways and fibrosis of the lung,

4. Exacerbations incident to viral infections, including RSV,

5. Progressive loss of pulmonary function,

6. Death within 3 to 5 years of diagnosis; >80% due to respiratory failure.

Therefore, therapeutic goals include:

1. Treatment of acute, disease modifying regimen targeted against Acute exacerbations of IPF induced by RSV

2. Reduce the need for mechanical ventilation in IPF

3. Reduce morbidity and mortality associated with mechanical ventilation in IPF

4/ Treat acute, disease modifying regimen targeted against Acute exacerbations of IPF not-induced by RSV

Cellular Populations At sites of pulmonary fibrosis and inflammation in IPF include: activated TH17⁺ epithelial cells, increased CCR6⁺ iDC's, increased CCR6⁺ Neutrophils, increased alternatively activated alveolar macrophages, increased local CCR7⁺ TH17⁺ fibroblasts and circulating fibrocytes, increased but anergized Tregs, increased TH17 leukocytes, increased circulating Cell Populations in IPF, and CXCR4⁺/CCR7⁺ fibrocytes.

Impact of CCR6 Blockade

By use of the antibodies of this invention, CCR6 blockade can reduce one or more of CCR6⁺ iDC population and survival, IL-17 activated epithelial cell CCL2 expression, CCR iDC IL-8, CCL18 and CCL20 secretion, alternatively activated macrophage CCL-18 responses, CCR6⁺ Th17 cell population and survival, CCR6⁺ neutrophil population and survival, CCR6⁺ mediated fibrocyte and Th17 cell migration to local foci of immuno-fibrosis, and/or CCR6⁺ Th17 mediated cell trafficking, inflammation and fibrosis.

Detection of Natively Configured CCR6

In certain embodiments of this invention, anti-CCR6 antibodies can be useful for detection of expressed CCR6 in native configuration. Prior methods of determining expression of CCR6 inadequately identify non-natively configured CCR6, and as such, may misrepresent the true amount of such CCR6 in a particular state. For example, RNA arrays and PCR assays (including quantitative PCR or “qPCR”) measure only the mRNA for CCR6 and do not reflect expression of the mature protein. Because CCR6 and other GPCRs are multispanning membrane proteins, misfolding of nascent protein chains may be important aspects of loss of CCR6 function and may lead to pathological conditions.

Additionally, anti-CCR6 antibodies raised against non-natively configured CCR6 may not detect mis-folded or mis-inserted CCR6 into cell membranes. Thus, using the antibodies of this invention, better understanding of the CCR6 status of patients can be achieved. In certain aspects, use of antibodies of this invention along with more routine analyses (qPCR, RNA arrays, and/or prior art antibodies) can shed light upon the functional state of a cell's CCR6 status.

### Physicochemical Properties of Monoclonal Antibodies Against Human CCR6

For a diagnostic, prognostic, and/or therapeutic use of antibodies, it can be desirable that the antibodies have physicochemical properties suitable for manufacture, formulation, packaging, and storage. Therefore antibodies of this invention can be easily manufactured, using methods known in the art. We also found that antibodies of this invention are stable in the face of changes in temperature are resistant to protease degradation, and do not undesirably degrade with time.

Compositions Containing Human Monoclonal Antibodies of this Invention

Antibodies of this invention can be formulated in a variety of ways to produce liquid solutions, suspensions, packaged into liposomes, attached to beads, or other types of compositions for therapeutic uses. For example, antibodies of this invention can be placed in a physiologically compatible solvent (e.g., phosphate buffered saline having physiologically compatible osmotic pressure, etc.). Additionally, antibodies may be formulated with other agents, including lipids; detergents, solubilizing agents, or other materials. Table 1 below shows some formulations of antibodies that can be used with antibodies to CCR6.

TABLE 1
Formulations for Antibody Drugs

| | | | Powder | Stock | | | | |
|---|---|---|---|---|---|---|---|---|
| Commere. | | Route of | or | Conc. | | | NaCl | |
| Name | Type | Admin. | Solution | mg/mL | pH | Buffer | mg/mL | Other Components/ |
| ABThrax | Anti-B, | | | | | | | |
| | anthrasis PA; | | | | | | | |
| | Human IgG1 | | | | | | | |
| Actemra | Anti-IL6R; | IV | Solution. | 20 | ~6.5 | Na | 0 | Disodium phosphate |
| | Humanized | | | | | Phos. | | dodecahydrate and sodium |
| | IgG1 | | | | | | | dihydrogen phosphate |
| | | | | | | | | dehydrate (as a 15 mM |
| | | | | | | | | phosphate buffer), |
| | | | | | | | | polysorbate 80 (0.5 |
| | | | | | | | | mg/mL), and sucrose (50 |
| | | | | | | | | mg/mL). |
| Numax | Anti-RSV; | | | | | | | |
| | Humanized | | | | | | | |
| | IgG1 | | | | | | | |
| Prolia | Anti-RANK- | SubCut | Solution | 60 | 5.2 | Na-Ac | 0 | Each 1 mL single-use |
| | L; Human | | | | | | | prefilled syringe of Prolia |
| | IgG2 | | | | | | | contains 60 mg |
| | | | | | | | | denosumab (60 mg/mL |
| | | | | | | | | solution), 4.7% sorbitol, |
| | | | | | | | | 17 mM acetate, 0.01% |
| | | | | | | | | polysorbate 20, NaOH. |
| Arzerra | Anti-CD20; | IV | Solution | 20 | 6.5 | Na-Cit | 5.85 | 8.55 mg/mL sodium |
| | Human IgG1 | | | | | | | citrate and 0.195 mg/mL |
| | | | | | | | | citric acid monohydrate as |
| | | | | | | | | buffering agents, 5.85 |
| | | | | | | | | mg/mL sodium chloride as |
| | | | | | | | | an isotonic agent. |
| Stelara | Anti-IL12/23; | SC | Solution | 90 | 5.7- | L-his | 0 | L-bistidine and L-histidine |
| | Human IgG1 | | | | 6.3 | HCl | | monohydrochloride |
| | | | | | | | | monohydrate (1 mg/mL), |
| | | | | | | | | Polysorbate 80 (0.04 |
| | | | | | | | | mg/mL), and sucrose (76 |
| | | | | | | | | mg/mL). |
| Ilaris | Anti-IL1β; | SC | Powder | 150 | N/A | L-his | 0 | 92.38 mg/mL sucrose, and |
| | Human IgG1 | | | | | HCl | | 0.60 mg/mL polysorbate |
| | | | | | | | | 80. L-histidine and L- |
| | | | | | | | | histidine hydrochloride |
| | | | | | | | | monohydrate are used to |
| | | | | | | | | adjust and buffer pH. |
| Simponi | Anti-TNFa; | SC | Solution. | 100 | 5.5 | L-his | 0 | 0.88 mg/mL L-histidine |
| | Human IgGI | | | | | HCl | | and L-histidine |
| | | | | | | | | monohydrochloride |
| | | | | | | | | monohydrate, 41 mg/mL |
| | | | | | | | | sorbitol, 0.16 mg/mL |
| | | | | | | | | polysorbate 80 |
| Cimzia | Anti-TNFα; | SC | Solution. | 200 | 4.7 | Na-Ac | 7.31 | 1.36 mg/mL sodium |
| | Hu-manized | | | | | | | acetate |
| | Fab pegyl. | | | | | | | |
| Soliris | Anti-C5; | IV | Solution. | 10 | 7.0 | Na | 8.77 | 0.46 mg/mL sodium |
| | Humanized | | | | | Phos. | | phosphate monobasic, |
| | IgG2/4 | | | | | | | 1.78 mg/mL sodium |
| | | | | | | | | phosphate dibasic, 0.22 |
| | | | | | | | | mg/mL polysorbate 80 |
| | | | | | | | | (vegetable origin) |
| Vectibix | Anti-EGFR; | IV | Solution. | 20 | 5.8 | Na-Ac | 5.80 | 6.8 mg/mL sodium acetate |
| | Human IgG2 | | | | | | | |
| Lucentis | Anti-VEGF; | Intra | Solution. | 10 | 5.5 | L-his | 0 | 10 mM histidine HCl, |
| | Humaniz | ocular | | | | HCl | | 10% α,α-trehalose |
| | IgG1 Fab | | | | | | | dihydrate, 0.01% |
| | | | | | | | | polysorbate 20 |
| Tysabri | Anti-α4 | IV | Solution | 20 | 6.1 | Na | 0.8 | 1.13 mg/mL sodium |
| | integrin; | | | | | Phos. | | phosphate, monobasic, |
| | Humanized | | | | | | | monohydrate; 0.48 mg/mL |
| | IgG4 | | | | | | | sodium phosphate, |
| | | | | | | | | dibasic, heptahydrate; 0.2 |
| | | | | | | | | mg/mL polysorbate 80 |
| Avastin | Anti-VEGF; | IV | Solution. | 25 | 6.2 | Na | 0 | 60 mg/mL α,α-trehalose |
| | Humanized | | | | | Phos. | | dihydrate, 5.8 mg/mL Na. |
| | IgG1 | | | | | | | phosphate (monobasic, |
| | | | | | | | | mono-hydrate), 1.2 |
| | | | | | | | | mg/mL Na phosphate |
| | | | | | | | | (dibasic, anhydrous), 0.4 |
| | | | | | | | | mL polysorbate 20 |
| Erbitux | Anti-EGFR; | IV | Solution | 2 | 7.0- | Na | 8.48 | 1.88 mg/mL sodium |
| | Chimeric | | | | 7.4 | Phos. | | phosphate dibasic |
| | IgG1 | | | | | | | heptahydrate, 0.41 mg/mL |
| | | | | | | | | sodium phosphate |
| | | | | | | | | monobasic monohydrate |
| Raptiva | Anti-CD11a; | SC | Powder | 100 | 6.2 | L-his | 0 | 98.56 mg/mL sucrose, |
| | Humanized | | | | | HCl | | 5.44 mg/mL L-histidine |
| | IgG1 | | | | | | | hydrochloride |
| | | | | | | | | monohydrate, 3.44 mg/mL |
| | | | | | | | | L-histidine and 2.4 |
| | | | | | | | | mg/mL polysorbate 20 |
| Bexxar | Anti-CD20; | IV | Solution. | 14 | 7.2 | Na | 145 | 10% (w/v) maltose, 10 |
| | Murine IgG2a | | | | | Phos, | mM | mM phosphate |
| Xolair | Anti-IgE; | SC | Powder | 125 | N/A | L-his | 0 | L-histidine (1.5 mg/mL), |
| | Humanized | | | | | HCl | | L-histidine hydrochloride |
| | IgG1 | | | | | | | monohydrate (2.33 |
| | | | | | | | | mg/mL), polysorbate 20 |
| | | | | | | | | (0.42 mg/mL), sucrose |
| | | | | | | | | (121.3 mg/mL) |
| Humira | Anti-TNFα; | SC | Solution | 50 | 5.2 | Phosph | 6.16 | 0.86 mg/mL monobasic |
| | Human IgG1 | | | | | Citrate | | sNa phosphate dihydrate, |
| | | | | | | | | 1.525 mg/mL dibasic Na |
| | | | | | | | | phosphate dihydrate, 03 |
| | | | | | | | | mg/mL, Na citrate, 1.3 |
| | | | | | | | | mg/mL citric acid |
| | | | | | | | | monohydrate, 12 mg/mL |
| | | | | | | | | mannitol, 1.0 mg/mL |
| | | | | | | | | polysorbate 80, NaOH to |
| | | | | | | | | adjust pH. |
| Zevalin | Anti-CD20; | IV | Solution | 1.6 | N/A | N/A | 9.0 | |
| | Murine IgG1- | | | | | | | |
| | tiuxetan conj. | | | | | | | |
| Campath- | Anti-CD52; | IV | Solution | 30 | 6.8- | Na-K | 8.0 | 1.44 mg/mL dibasic Na. |
| 1H | Humanized | | | | 7A | Phos. | | phosphate, 0.2 mg/mL |
| | IgG1 | | | | | | | KCl, 0.2 mg/mL |
| | | | | | | | | monobasic K phosph-ate, |
| | | | | | | | | 0.1 mg/mL polysorbate |
| | | | | | | | | 80, 0.0187 mg/mL |
| | | | | | | | | disodium edetate |
| | | | | | | | | dihydrate [EDTA]. |
| Mylotarg | Anti-CD33; | IV | Powder | 1.0 | N/A | Na | N/A | Conj. with calicheamicin |
| | Humanized | | | | | Phos. | | N-acetyl-gamma |
| | IgG4-Conjug. | | | | | | | calicheamicin via a |
| | | | | | | | | bifunctional linker. 50% |
| | | | | | | | | of the antibody loaded |
| | | | | | | | | with 4-6 moles |
| | | | | | | | | calicheamicin per mole of |
| | | | | | | | | antibody. The remaining |
| | | | | | | | | 50% of the antibody is not |
| | | | | | | | | linked to the |
| | | | | | | | | calicheamicin derivative, |
| | | | | | | | | dextran 40; sucrose; |
| | | | | | | | | sodium chloride; |
| | | | | | | | | monobasic and dibasic |
| | | | | | | | | sodium phosphate. |
| Herceptin | Anti-HER-2; | IV | Powder | 21 | ~6 | L-his | 0 | 20 mg/mL α,α-trehalose |
| | Humanized | | | | | HCl | | dihydrate, 0.495 mg/mL L- |
| | IgG1 | | | | | | | histidine HCl, 0.32 mg/mL |
| | | | | | | | | L-histidine, and 0.09 |
| | | | | | | | | mg/mL polysorbate 20, |
| | | | | | | | | 1.1% benzyl alcohol |
| Remicade | Anti-TNFα: | IV | Powder | 10 | 7.2 | Na | 0 | 50 mg/mL sucrose, 0.05 |
| | Chimeric | | | | | Phos. | | mg/mL polysorbate 80, |
| | IgG1 | | | | | | | 0.22 mg/mL monobasic |
| | | | | | | | | sodium phosphate, |
| | | | | | | | | monohydrate, and 0.61 |
| | | | | | | | | mg/mL dibasic sodium |
| | | | | | | | | phosphate dihydrate. |
| Synagis | Anti-RSV; | IM | Soltn. | 100 | 6.0 | His/Gly | 0 | 3.9 mg/mL histidine, 0.1 |
| | Humanized | | | | | HCl | | ing/mL glycine, and 0.5 |
| | IgG1 | | | | | | | mg/mL chloride. |
| Simulect | Anti-IL2R: | IV | Powder | 4 | N/A | Na-K | 0.32 | 1.44 mg/mL monobasic K |
| | Chimeric | | | | | Phos | | phosphate, 0.2 mg/mL |
| | IgG1 | | | | | . | | disodiurn hydrogen |
| | | | | | | | | phosphate (anhydrous, 4 |
| | | | | | | | | mg/mL sucrose, 16 mg/mL |
| | | | | | | | | mannitol, and 8 mg/mL |
| | | | | | | | | glycine |
| Zenapax | Anti-IL2R; | IV | Soltn. | 5 | 6.9 | Na | 4.6 | 3.6 mg/mL, sodium |
| | Humanized | | | | | Phos. | | phosphate monobasic |
| | IgG1 | | | | | | | monohydrate, 11 mg/mL |
| | | | | | | | | sodium phosphate dibasic |
| | | | | | | | | heptahydrate, 0.2 mg/mL |
| | | | | | | | | polysorbate 80, HCl or |
| | | | | | | | | NaOH to adjust the pH. |
| Rituxan | Anti-CD20; | IV | Soltn. | 10 | 6.5 | Na-Cit | 9.0 | 7.35 mg/mL sodium |
| | Chimeric | | | | | | | citrate dihydrate, 0.7 |
| | IgG1 | | | | | | | mg/mL polysorbate 80 |
| Reopro | Anti- | IV | Soltn. | 2 | 7.2 | Na | 150 | 0.01 M sodium phosphate, |
| | GPIIb/IIIa; | | | | | Phos. | mM | 0.001% polysorbate 80 |
| | Chimeric | | | | | | | |
| | IgG1 Fab | | | | | | | |
| Na-Ac: | Sodium acetate | | | | | | | |
| Na-Cit: | Sodium citrate; | | | | | | | |
| Na-Phos. | Sodium phosphate | | | | | | | |

For NaCl, 150 mM is ~9 mg/mL

Additionally, antibodies of this invention can be used in diagnostic kits, which can contain antibodies, antibodies linked to streptavidin or biotin (for conjugation), solubilizing agents, mixing vials, and instructions for carrying out in vitro analysis of the presence of CCR6 in samples obtained from human beings or other animals that express CCR6. For example, biotinilation of anti-CCR6 antibodies using a commercial biotinilation reagent EZ-Link Sulfo-NHS-LC-Biotin (ThermoScientific, Catalog Number 21335) was performed as per the manufacturer recommendation and so derivatized antibodies displayed binding EC50 comparable to that for original antibodies. The biotinilated antibodies bound to CCR6 on PBMCs and CCR6-expressing reporter cell lines were further stained with Streptavidin-PE and cells were analyzed with fluorescence flow cytometer. Antibodies can also be linked to detectable tags (e.g., fluorescent tags) enabling their detection using a variety of analytic methods.

### Embodiments of the Invention

It can be readily appreciated that aspects of this invention can be implemented in numerous ways. Some embodiments are described herein below. It can also be appreciated that other embodiments can be carried out by persons of skill in the art without undue experimentation. All such embodiments are considered part of this invention.

Some embodiments include a fully human antibody against human chemokine receptor CCR6.

Other embodiments further comprise an antibody that selectively binds to human CCR6 and is in IgG1, IgG2, IgG3, or IgG4, or other immune globulin format Other embodiments of any one or more of the above embodiments can further comprise an antibody is essentially free of contaminants.

Additional embodiments of any of the above embodiments further comprise a fragment capable of specifically binding to human, mouse, and to cynomologus monkey CCR6.

Additional embodiments of the above can comprise an antibody, antibody fragment thereof that binds to CR6 with a binding affinity of between about 1 nM and about 100 nM.

Further embodiments of the above can include a pharmaceutical composition comprising a fully human antibody against human CCR6; and a pharmaceutically acceptable carrier or excipient.

Still further embodiments of the above include an antibody of Claim 1, wherein said antibody is essentially free of contaminants.

Additional embodiments of one or more of the above comprise a fully human antibody fragment, said fragment capable of specifically binding to human CCR6.

Additional embodiments include a library of fully human anti-CCR6 antibodies, of scFv or Fab fragments of said CCR6 antibodies.

Certain embodiments of one or more of the above embodiments include a fully human anti-CCR6 antibody, scFv or Fab fragment having a CDR3 HC sequence or a CDR3 LC sequence selected from any of Tables 3 through 41.

Additional embodiments of one or more of the above embodiments include a fully human anti-CCR6 antibody having a CDR3 sequence selected from Tables 3 through 41.

Yet additional embodiments of the above embodiments include an antibody of against human CCR6 in IgG1 format.

Other embodiments of one or more of the above embodiments comprise an antibody against human CCR6, said antibody in IgG4 format.

Still further embodiments of one or more of the above embodiments comprise an antibody fragment, comprising an scFv or Fab fragment of an antibody of Claim 1, where said antibody fragment specifically binds to human CCR6 with an affinity of between about 1 nM to about 100 nM.

Further embodiments of one or more of the above embodiments include a composition comprising an antibody or antibody fragment of any preceding embodiment, further comprising a physiologically compatible solution.

Additional embodiments include a composition of any preceding embodiment, father comprising one or more physiologically compatible excipients or binders.

Other embodiments include methods for inhibiting an abnormal effect of CCR6, comprising administering to a mammal in need thereof an antibody or fragment thereof of a human anti-CCR6 antibody.

Further embodiments include methods of any preceding embodiment, wherein said fully human antibody against CCR6 is an antibody fragment capable of specifically binding to human CCR6 receptor.

Still further embodiments include methods of any preceding embodiment, where said antibody fragment is m scFv fragment or a Fab fragment of said antibody having a binding affinity of about 1 nM to about 100 nM.

Additional embodiments include use of a fully human antibody against CCR6, an scFv or Fab fragment thereof in the manufacture of a medicament useful for treating a disorder in an animal caused by ligand-mediated overactivity of CCR6.

Other embodiments include any of the preceding uses, where said antibody or fragment thereof is selected from any of Tables 3-41.

Alternative embodiments include any of the preceding uses, where the abnormal effect of CCR6 is an abnormal inflammatory response.

Further alternative embodiments of any of the preceding uses include uses where said abnormal effect is a fibrotic disease, inflammation, or infection.

In certain alternative uses of any of the preceding uses, said abnormal effect is a pulmonary disease.

In some embodiments of any of the uses above, said pulmonary disease is Respiratory Syncytial Virus (“RSV”) induced fibrosis, Idiopathic Pulmonary Fibrosis (“IPF”), Chronic Obstructive Pulmonary Disease (“COPD”), severe Asthma, Fibrotic Sequellae of Acute Lung Injury, or Adult Respiratory Distress Syndrome (ARDS)

Additional uses of any of the above embodiments include uses where said abnormality is cancer.

Further uses include a kit, comprising one or more fully human antibodies or fragments thereof against human CCR6 receptor, said antibodies or fragments thereof capable of selectively binding to said CCR6 receptor, a solution comprising a pharmaceutically acceptable excipient; and instructions for use.

Yet further uses include methods for determining the presence of CCR6 receptor in a biological sample, comprising providing a biological sample, exposing said sample to an antibody or fragment thereof that specifically binds to CCR6; and detecting the presence of said antibody bound to said biological sample.

Additional embodiments include methods of manufacturing a fully human antibody or fragment thereof against human CCR6, comprising the steps producing a codon-optimized DNA plasmid encoding human CCR6, expressing said plasmid an a cell capable of producing CCR6, extracting said CCR6 from said cell using a detergent-containing solution.

Further embodiments of any of the above embodiments include attaching said CCR6 antibody or fragment thereof to a bead;

Additional embodiments include producing a library of human IgG antibodies or fragments thereof; and selecting from said library, antibodies that bind to human CCR6.

Still further embodiments include any preceding embodiment further including a kit for detecting human CCR6, comprising a fully human antibody or a fragment thereof directed against human CCR6; a vial for preparing said antibody for use, solutions for use in an in vitro assay; and instructions for use.

Additional embodiments of the above further include biotinylating said antibody or fragment thereof.

### EXAMPLES

The following examples are presented to illustrate aspects and embodiments of this invention. As such, they are not intended to limit the scope of the invention. Rather, persons of skill in the art can use the descriptions and teachings herein to create, modify or produce other human antibodies and uses thereof without undue experimentation. All such embodiments are considered part of this invention.

### Example 1

### Binding of Anti-CCR6 Antibodies to Cell Lines

To determine whether fully human antibodies of this invention bind to CCR6 or other GPCRs, we carried out studies using a series of cell lines in culture. The cell lines used were:

A. CHO-K1 (Chinese Hamster Ovary cells, ATCC Cat #CCL-61);

B. BHK-21 (Syrian Hamster Fibroblasts, ATCC Cat #CCL-10);

C. CF2Th (Canine Thymocytes, ATCC Cat #CRL-1430);

D. R1610 (Chinese Hamster Lung Fibroblasts, ATCC Cat #CRL-1657); and

E. HEK-293T (Human Embryonic Kidney cells, Cat #CRC-1573).

The lines were also adapted to stably express different G-Protein Coupled Receptors (GPCRs). The mammalian cells adapted to stable expression of GPCRs included cell lines expressing human and orthologous GPCRs that also belong to the subclass of chemokine receptors and are closely related to CCR6; the cell lines were generated and tested for the expression of respective GPCRs using staining with commercially available anti-respective-GPCR conjugates with fluorescent moiety PE:

R1610-human CXCR1 (Extracellular staining with anti-human CXCR1 mouse antibody conjugated to PE. BD Pharmigen, Cat. #555940).

Cf2Th-human CXCR2 (Extracellular staining with anti-human CXCR2 mouse antibody conjugated to PE. BD Pharmigen Cat. #555933).

R1610-human CXCR3 (Extracellular staining with anti-human CXCR3 mouse antibody conjugated to PE. R&D Systems, Cat. #FAB160P).

Cf2Th-human CXCR4 (Extracellular staining with anti-human CXCR4 CD1864/12G5 mouse antibody conjugated to PE. BD Pharmigen, Cat. #557145).

CHO-hmaen CXCR5 (Extracellular staining with anti-human CXCR5 mouse antibody conjugated to PE. R&D Systems, Cat. #FAB190P).

CHO-human CXCR6 (Extracellular staining with anti-human CXCR6 mouse antibody conjugated to PE. R&D Systems, Cat. #FAB699P).

CHO-human CXCR7 (Extracellular staining with anti-human CXCR7 mouse antibody conjugated to PE. R&D Systems, Cat. #FAB42271P).

CHO-human CCR3 (Extracellular staining with anti-human CCR3 mouse antibody conjugated to PE. BD Pharmigen, Cat. #558165).

CHO-human CCR4 (Extracellular staining with anti-human CCR6 mouse antibody conjugated to PE. R&D Systems, Cat. #FAB1567P).

CHO-human CCR5 (Extracellular staining with anti-human CCR5 mouse antibody conjugated to PE. BD Pharmigen, Cat. #556042).

R1610-human CCR6 (Extracellular staining with anti-human CCR6 mouse antibody conjugated to PE. R&D Systems, Cat. #FAB195P).

CHO-human CCR6 (Extracellular staining with anti-human CCR6 mouse antibody conjugated to PE. R&D Systems, Cat. #FAB195P).

CHO-cyno CCR6 (Extracellular staining with anti-human CCR6 mouse antibody conjugated to PE. R&D Systems; Cat. #FAB195P).

CHO-mouse CCR6 (Intracellular staining with Streptavidin PE R&D Systems, Cat. #F0040).

CHO-human CCR7 (Extracellular staining with anti-human CCR7 mouse antibody conjugated to PE. BD Pharmigen, Cat. #12-1979-42).

R1610-human CCR7 (Extracellular staining with anti-human CCR7 mouse antibody conjugated to PE. BD Pharmigen, Cat. #12-1979-42).

CHO-mouse CCR7 (Extracellular staining with anti-human CCR7 mouse antibody conjugated to PE. BD Pharmigen, Cat. #12-1979-42).

R1610-human CCR9 (Extracellular staining with anti-human CCR9 mouse antibody conjugated to PE. BD Pharmigen, Cat. #557975).

CHO-human CCR10 (Extracellular staining with anti-human CCR10 mouse antibody conjugated to PE. R&D Systems, Cat. #FAB3478P).

CHO-cyno CXCR3 (Extracellular staining with anti-human CXCR3 mouse antibody conjugated to PE. R&D Systems, Cat. #FAB160P).

The histogram presented in FIG. 1 depicts the Mean Fluorescence Intensity (MFI) obtained by staining of the above cell lines with their respective over-expressed GPCR antibody-PE conjugates; although not shown, cells that did not overexpress that GPCR displayed much smaller MPI that was below 10 units. Thus, the set of cell lines above indeed each expressed their respective GPCR and therefor was useful for assaying the specificity of binding of anti-CCR6 antibodies of this invention.

In the assay, the following cell staining procedure was carried out 5,000-10,000 cell suspension in 10 μl FACS buffer (1×PBS, 2.0% FBS, 0.2% sodium azide) was mixed with 10 μl of 200 nM the corresponding anti-CCR6 MAB and incubated on ice for 30 min. Washing step—after the incubation 150 μl of FACS buffer was added to the cell sample, the samples were mixed gently by up-down pipetting the cell suspension. Then the samples were centrifuged at 1100 rpm for 5 minutes, and supernatants were removed. Washing step was repeated ones. Then 10 μl of anti-human PE-(Fab)2 form Jackson Immuno Research Lab. #709-116-098 diluted 40 times in FACS buffer were added to the calls and the cells were re-suspended by up-down pipetting. After 20 min. incubation on ice in the dark the washing step was repeated twice. The washed cells were mixed with 100 μl of FIX buffer (0.5% Paraformaldehyde solution in PBS). Fixed samples were stored in the dark on ice and analyzed by FACS on Guava PCA-96 flow cytometer.

### Example 2

### Preparation of CCR6 Target Presentation Materials CCR6-Golik™ of this Invention

The manufacturing of CCR6-Golik particles was comprised of the following protocols of the present invention: Paramagnetic particles, which can be M-280 Tosylactivated Dynabeads produced by Dynal Biotech Inc. chemically derivatized with a capture agent, using protocol provided by the beads manufacturer, the capture agent can be an anti-protein tag antibody that is capable of selective binding its respective tag, or streptavidin that can bind a known peptide called Strep-Tag™. Either of the tags was genetically attached at the C-terminus of a given membrane protein. While the following describes the protocol for preparing CCR6-Golik TPM using Streptavidin-coupled Dynabeads already having their surface derivatized with streptavidin, other tag-capture agent pairs can be used. Human CCR6 having a Strep-tag capable of binding to its respective capture agent, Streptavidin on the bead surface was over-expressed in a mammalian cell culture. Cells that over-expressed the human CCR6 protein were harvested, and the membranes of the cells were solubilized in a detergent, or mixture of detergents, or in mixture of detergents also containing lipids (e.g. phosphatidylcholine, phosphatidylserine phosphatidethanolamine, or lipid mixtures isolated form tissues or plants, or cholesterol hemisuccinate (CHS). Appropriate solubilization Buffers are described in Example 5 below. Solubilization was performed by pelleting cells down and adding a detergent mixture (˜3-5 volumes of cell pellet; typically, 10̂7 cells pellet was used, which was ˜100 uL as a pellet, and 300-500 uL of a detergent mixture was added). After 1 hour solubilization an ice, the solubilized material was collected as a supernatant after pelleting down debris by centrifugation. Capturing of CCR6 from the solubilized material was then achieved by adding to an Eppendorf tube containing the above solubilized material a suspension 50 uL of the commercial Streptavidin beads FACS-washed; then the tube was kept at 4° C. under slow rotation overnight. The solubilization and capturing of the CCR6 protein molecules via the Strep-tag on the Streptavidin-coupled Dynabeads™ was performed under varying a broad matrix of conditions that included various detergents in the presence and absence of the lipids.

The samples obtained using each of the matrix conditions were washed 2-3 times with FACS buffer, stained with commercial anti-human CCR6 antibodies conjugated with PE, and analyzed by Guava PCA-96 fluorescence cell flow cytometer measuring the MFI signal of the antibody-PE conjugate bound to so obtained beads. The beads so produced are TMP in the form of CCR6-Golik of this invention. The highest MFI was achieved using the following solubilization and capturing buffer (CCR6 Solubilization Buffer): 1% DDM, 20 mM Tris-HC, pH7.5, 100 mM Ammonium Sulfate, 10% Glycerol. In contrast to the MPL preparation, no step of addition of lipid in order to reconstitute the lipid bilayer was employed. Presumably the lipid bilayer was not reconstituted completely, and we found that only molecules of detergent—lipid mixture were needed to stabilize the protein structure. This mechanism is presented only as a possibility. Other mechanisms may be responsible, and all are considered part of this invention.

### Example 3

### Selections from Human Antibody Libraries

Phage-display libraries are among the most used technologies for generation and optimization of fully human antibodies (see Hoogenboom, H. R. Selecting and screening recombinant antibody libraries. Nature Biotechnol. 23, 1105-1116 (2005); Bradbury, A. R. & Marks, J. D. Antibodies from phage antibody libraries. J. Immunol. Methods 290, 29-49 (2004); and Fredericks, Z. L. et al. Identification of potent human anti-IL-1R I antagonist an bodies. Protein Eng. Des. Sel. 17, 95-106 (2004)).

Fully human antibodies against CCR6 were derived from Fab phage display libraries with diversity of about 10¹², in which each phage particle carries on its surface at least one and up to four molecules of Fab and in its genome complete genetic information about the amino acid composition of the Fab molecule. The diversity was achieved by introducing randomization into all three CDRs of the heavy chain, and into CDR3 of the light chain.

For selections, the CCR6-Golik presentation material was washed with FACS buffer and all selections were performed in FACS buffer. For each selection a freshly prepared CCR6-Golik TPM was used, upon quality control—MFI for the commercial anti-CCR6 antibody PE conjugate exceeding MFI of non-stained beads by at least 20 times.

The selections started with the initial depletion step, in which 10¹² phage particles in 1 ml FACS buffer were first incubated with Streptavidin-Dynabeads™ (pre-treated with solubilized material obtained from parental cells that did not expressed CCR6 in the same manner as described above for preparing the CCR6-Golik beads) under slow rotation at 4° C. for 30-60 min. Then the Streptavidin-Dynabead™ were removed, and the phage suspension was mixed with the suspension of CCR6-Golik beads (total volume ˜1 mL) and incubated under gentle rotation for 1 hour at 4° C.

The CCR6-Golik beads with bound phage particles were collected by washing non-specifically bound and file phage particles three times with FACS (1 ml washing volume each time) and three times with the CCR6 SB buffer (again, 1 ml washing volume each time), then one more time with PBS, after which the phage particles bound to the CCR6-Golik beads were eluted by acidic treatment using 0.5 mL Acidic Buffer (0.1 M Glycine-HCl, pH 2.2). Beads were removed. The acid eluted phage suspension was neutralized with 1.0M Tris-HCl, pH 8.0. Phage then was propagated by infecting E. Coli bacteria, collected by precipitation with 2.5M NaCl, 20% PEG (6,000 MW), and used in the next round of selections.

In some selection rounds, CCR6-expressing cells (about 10⁶-10⁷ cells per selection) instead of CCR6-Golik were employed as TPM. In the initial depletion step in these selections respective parental cells that did not express CCR6 were mixed with other cell lines that expressed other GPCRs but again not CCR6 in equal proportion (total about 10⁷ cells of 3 different types per selection), that followed by incubation of the cells with phage particles (1 mL total volume) under mild rotation for 20 min at 4° C., after which cells were removed by centrifugation and supernatant was mixed again with fresh cell mixture; total 3 cycles of incubation of phage with cells were performed. The depleted phage suspension was mixed with cells expressing CCR6, incubated for 1 hour under gentle rotation, cells with bound phage were washed with 1 mL FACS buffer 3 to 5 times, and then one time with PBS, and phage was acid eluted and propagated as described above. The total 3 to 4 rounds of selections were performed to obtain the antibodies of this invention. The protocol combining selections on CCR6-Golik and cells constitute an embodiment of the present invention. Also, obtained outputs of the 3^{rd} and 4^{th} rounds of selections contained about 10⁶ phage particles, of which roughly 1% to 10% carry Fab molecules that bind CCR6. Only a fraction of these outputs (roughly 10,000 clones) were screened and a large number of anti-CCR6 fully human antibodies remains in these outputs untapped. The outputs thus are the enriched libraries of anti-CCR6 potential binders of this invention.

### Example 4

### Construction and Expression of Codon-Optimized and Natural CCR6 Genes

A number of service providers, such as for example Genewiz (http://www.genewiz.com/public/gene-synthesis.aspx) currently offer commercial service of synthesis of protein genes, and the human CCR6 gene was synthesized using such a service. The cynomolgus monkey and mouse CCR6 ortholog genes were derived from commercially available libraries—cDNA library for mouse and total RNA library for Macaca Fascicularis (Cynomolgus) monkey by means of convention molecular biology techniques.

### Example 5

### Solubilization Buffers

Detergents were used as components of solubilization buffers. The detergents, with abbreviations and critical micelle concentrations in parentheses, were n-octyl-β-D-glucopyranoside (23.4 mM), n-decyl-β-D-maltoside (1.8 mM), n-dodecyl-β-D-maltoside (DDM; 0.17 mM), cyclohexyl-butyl-β-D-maltoside (Cymal™-4; 7.6 mM), cyclohexyl-pentyl-β-D-maltoside (Cymal™-6; 0.56 mM), cyclohexyl-heptyl-β-D-maltoside (Cymal™-7; 0.19 mM), cyclo-hexylpropanoyl-N-hydroxyethylglucamide (108 mM), cyclohexylbutanoyl-N-hydroxyethylglucamide (35 mM), cyclohexylpentanoyl-N-hydroxyethylglucamide (11.5 mM), N-octylphosphocholine (Fos-Choline™ 8; 114 mM), N-decylphosphocholine (Fox-Choline™ 10; 1 mM), N-dodecylphosphocholine (Fos-Choline™ 12; 1.5 mM), N-tetradeylphosphocholine (Fox-Choline™ 14; 0.12 mM), Triton X-100 (0.02 mM), CHAPS (8 mM), Nonidet P-40 (0.02 mM), and diheptanoyl-phosphocholine (DHPC; 1.4 mM). All detergents were purchased from Anairace (Maumee, Ohio) except DHPC, which was purchased from Avanti Polar Lipids (Alabaster, Ala.).

### Example 6

### Isolation and Characterization of Binding of Anti-CCR6 Antibodies

Anti-CCR6 antibodies of this invention were produced in transiently transfected CHO cells in serum-free medium in IgG1 format and harvested on day 5 or 6 according to the protocol licensed from Canadian Research Council and purified under endotoxin-free condition using affinity chromatography on Protein A, acidic elution followed by immediate neutralization to pH6.0 and dialysis against a storage buffer. According to the SDS-PAGE, the purified antibodies were 95% pure.

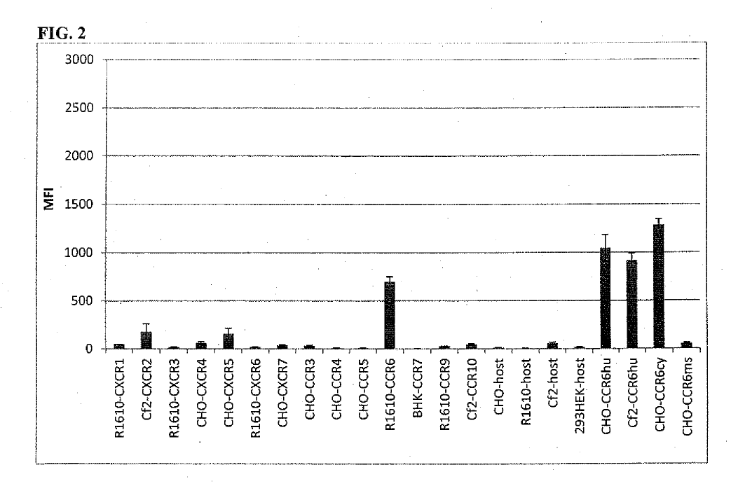

Sixteen histograms are shown in FIGS. 2-17 demonstrating the signal collected from the cells expressing human CCR6, cyno CCR6, and mouse CCR6 and different other GPCRs (used as controls). Cells have been incubated with fully human anti-human CCR6 antibodies in IgG1 format. After washing out residual unbound antibodies the cells were incubated with commercial anti-human Fc antibodies conjugated to the fluorescent dye phycoerythrin (IgG-PE). In more details, the cell staining procedure was: 5000-1000 cell suspension in 10 μl FACS buffer (1×PBS, 2.0% FBS, 0.2% sodium azide) was mixed with 10 μl of 200 nM the corresponding anti-CCR6 MAB and incubated on ice for 30 min. Washing step—after the incubation 150 μl of FACS buffer was added to the cell sample, the samples were mixed gently by up-down pipetting the cell suspension. Then the samples were centrifuged at 1100 rpm for 5 minutes, and supernatants were removed. Washing step was repeated ones. Then 10 μl of anti-human PE-(Fab)2 form Jackson Immuno Research Lab. #709-116-098 diluted 40 times in FACS buffer were added to the cells and the cells were re-suspended by up-down pipetting. After 20 min. incubation on ice in dark the washing step was repeated twice. The washed cells were mixed with 100 μl of FIX buffer (0.5% Paraformaldehyde solution in PBS). Fixed samples were stored in dark on ice and analyzed by FACS on Guava PCA-96 flow cytometer.

FIG. 2 depicts a graph of fluorescence of cells expressing human CCR6, or its cynomolgus monkey or mouse orthologs, or other GPCRs, in the presence of human antibody IgG1 MSM-R601 of this invention, and labeled with anti-human antibody Fc-PE commercial conjugate. Columns 1-22 show results for the cells: (1) R1610-human CXCR1; (2) Cf2th-human CXCR2; (3) R1610-human CXCR3; (4) CHO-human CXCR4; (5) CHO-human CXCR5; (6) R1610-human CXCR6; (7) CHO-human CXCR7; (8) CHO-human CCR3; (9) CHO-human CCR4; (10) CHO-human CCR5; (11) R1610-human CCR6; (12) CHO-human CCR7; (13) R1610-human CCR9; (14) CHO-human CCR10; (15) CHO host cell line; (16) R1610 host cell line; (17) Cf2Th host cell line; (18) 293HEK host cell line; (19) CHO-human CCR6; (20) Cf2Th-human CCR6; (21) CHO-cyno[molgus monkey] CCR6; and (22) CHO mouse CCR6.

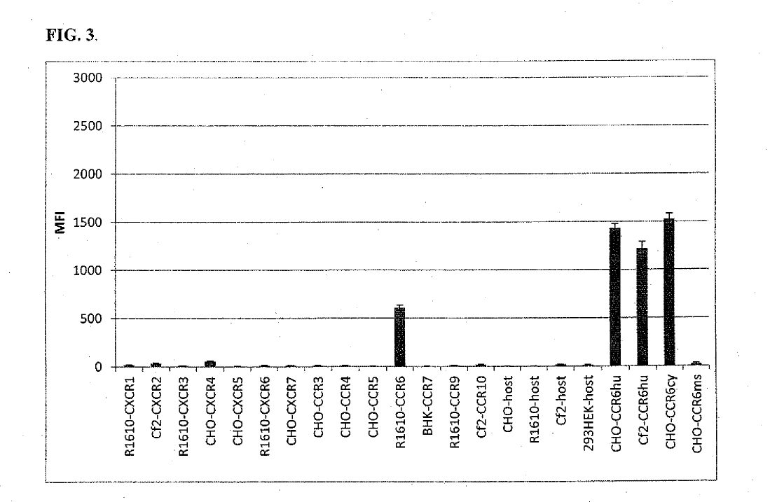

FIG. 3 depicts a graph of fluorescence of the cells as in FIG. 2 expressing CCR6 or other GPCRs, in the presence of human antibody IgG1 MSM-R602 of this invention and labeled with anti-human antibody Fc-PE commercial conjugate.

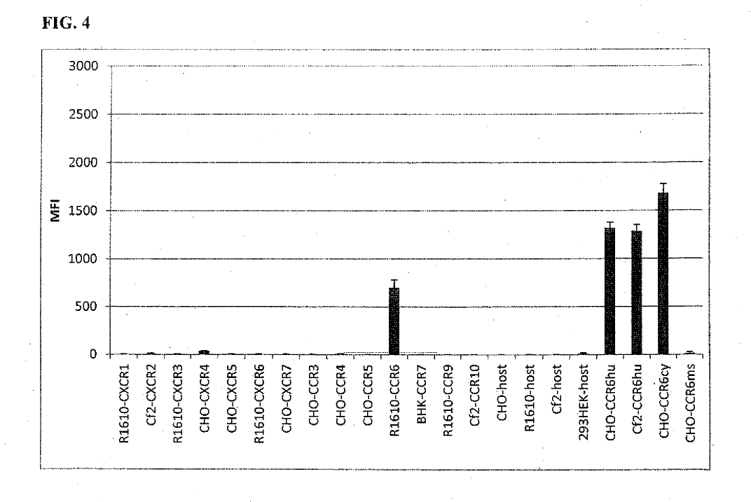

FIG. 4 depicts a graph of fluorescence of the cells as in FIG. 2 expressing CCR6 or other GPCRs, in the presence of human antibody IgG1 MSM-R604 of this invention and labeled with anti-human antibody Fc-PE commercial conjugate.

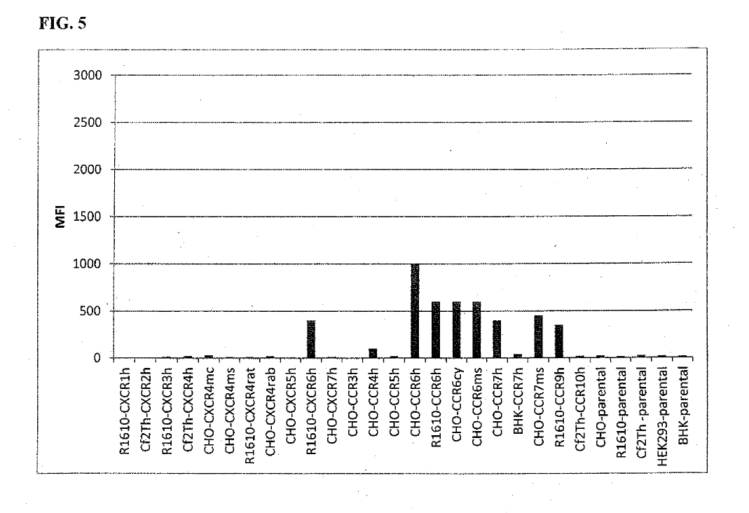

FIG. 5 depicts a graph of fluorescence of the cells as in FIG. 2 expressing CCR6 or other GPCRs, in the presence of human antibody IgG1 MSM-R605 of this invention and labeled with anti-human antibody Fc-PE commercial conjugate.

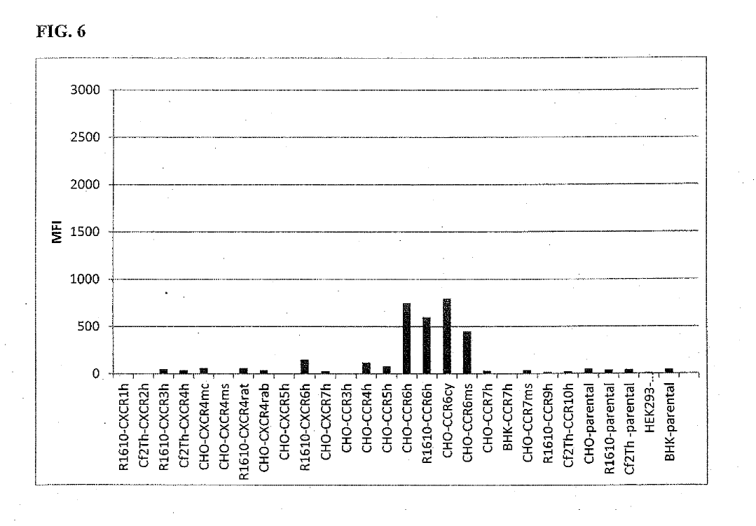

FIG. 6 depicts a graph of fluorescence of the cells as in FIG. 2 expressing CCR6 or other GPCRs, in the presence of human antibody IgG1 MSM-R606 of this invention and labeled with anti-human antibody Fc-PE commercial conjugate.

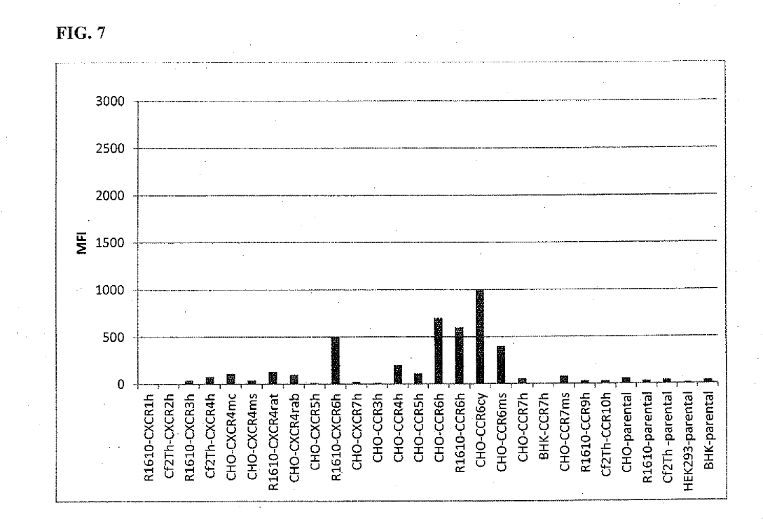

FIG. 7 depicts a graph of fluorescence of the cells as in FIG. 2 expressing CCR6 or other GPCRs, in the presence of human antibody IgG1 MSM-R607 of this invention and labeled with anti-human antibody Fc-PE commercial conjugate.

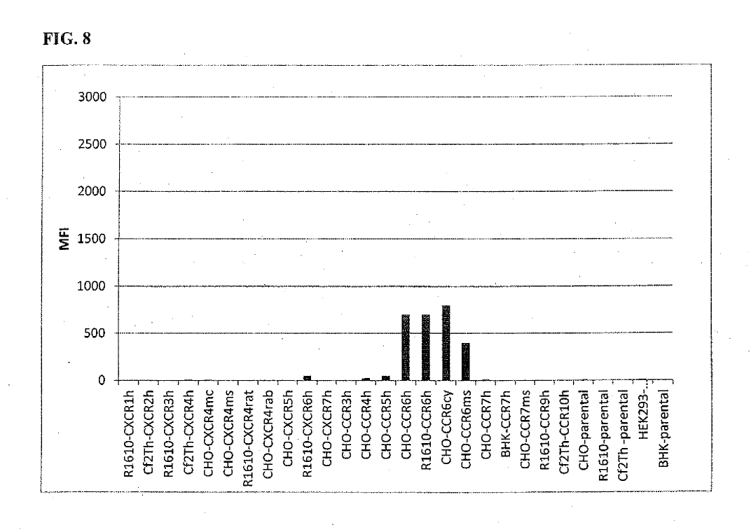

FIG. 8 depicts a graph of fluorescence of the cells as in FIG. 2 expressing CCR6 or other GPCRs, in the presence of human antibody IgG1 MSM-R608 of this invention and labeled with anti-human antibody Fc-PE commercial conjugate.

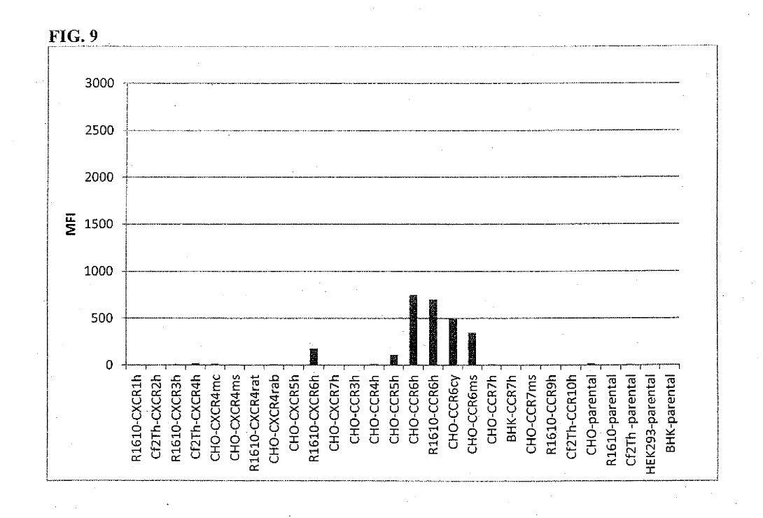

FIG. 9 depicts a graph of fluorescence of the cells as in FIG. 2 expressing CCR6 or other GPCRs in the presence of human antibody IgG1 MSM-R609 of this invention and labeled with anti-human antibody-PE commercial conjugate.

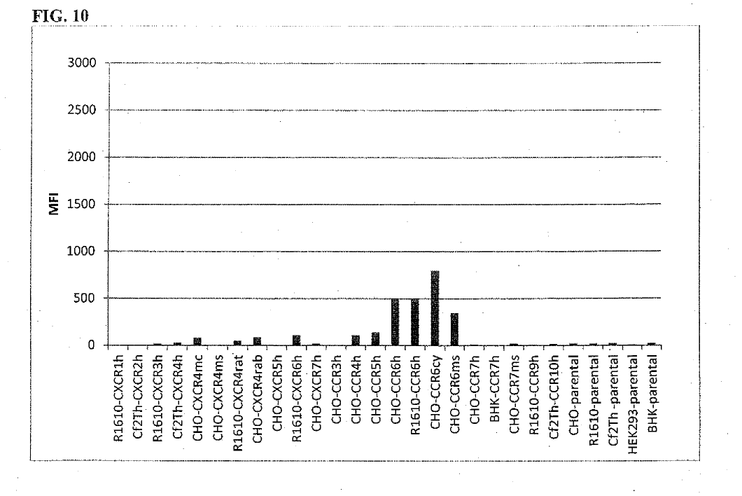

FIG. 10 depicts a graph of fluorescence of the cells as in FIG. 2 expressing CCR6 or other GPCRs, in the presence of human antibody IgG1 MSM-R612 of this invention and labeled with anti-human antibody Fc-PE commercial conjugate.

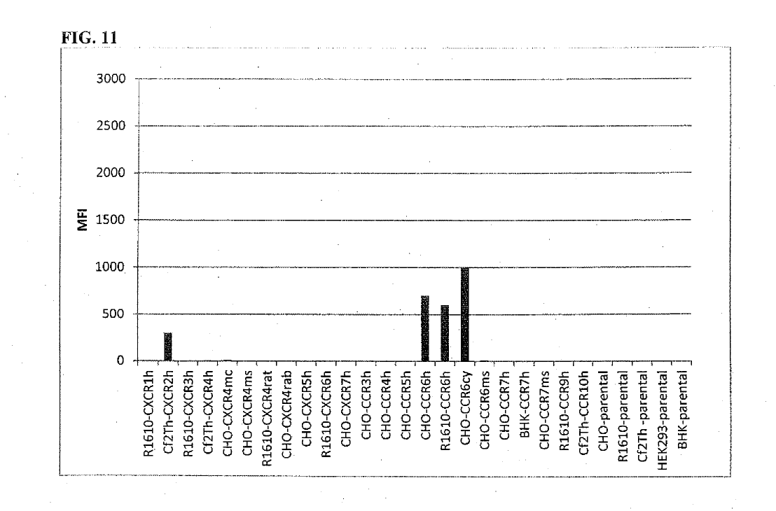

FIG. 11 depicts a graph of fluorescence of the cells as in FIG. 2 expressing CCR6 or other GPCRs, in the presence of human antibody IgG1 MSM-R614 of this invention and labeled with anti-human antibody Fc-PE commercial conjugate.

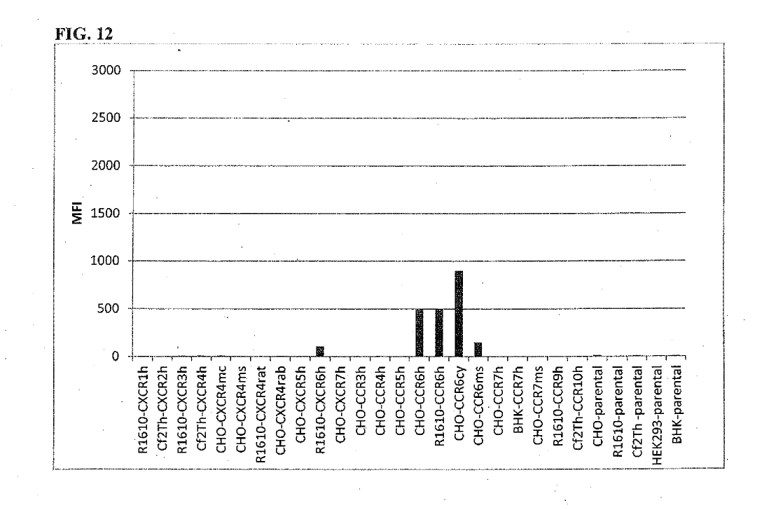

FIG. 12 depicts a graph of fluorescence of the cells as in FIG. 2 expressing CCR6 or other GPCR, in the presence of human antibody IgG1 MSM-R615 of this invention and labeled with anti-human antibody Fc-PE commercial conjugate.

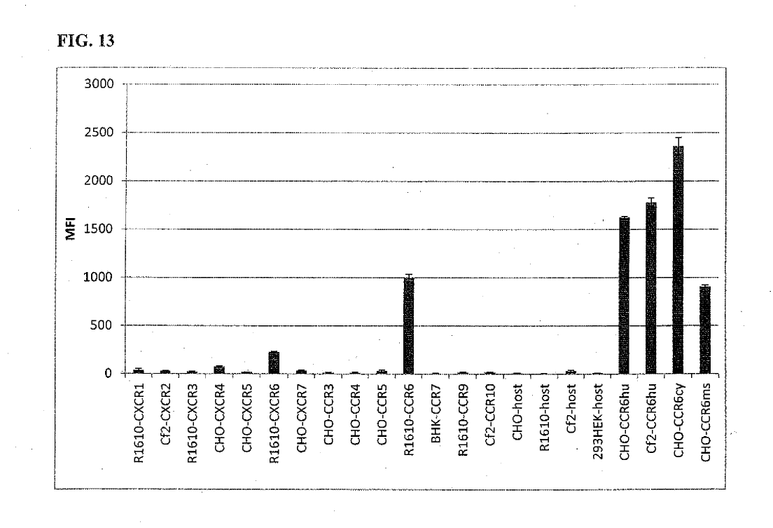

FIG. 13 depicts a graph of fluorescence of the cells as in FIG. 2 expressing CCR6 or other GPCRs in the presence of human antibody IgG1 MSM-R617 of this invention and labeled with anti-human antibody Fc-PE commercial conjugate.

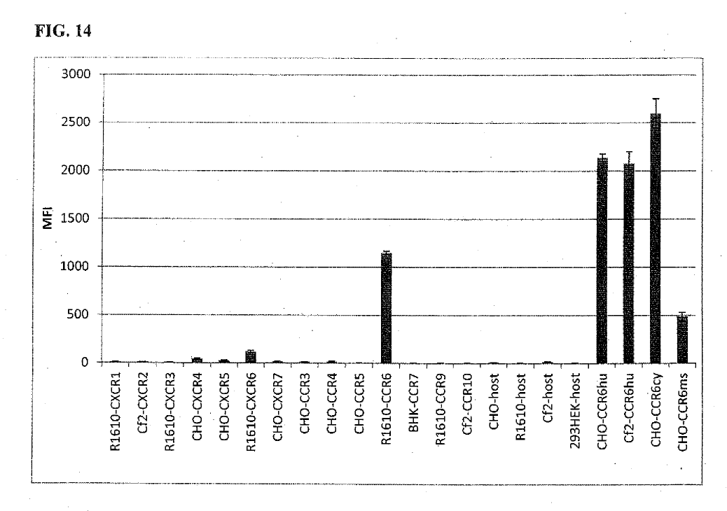

FIG. 14 depicts a graph of fluorescene of the cells as in FIG. 2 expressing CCR6 or other GPCRs, in the presence of human antibody IgG1 MSM-R619 of this invention and labeled with anti-human antibody Fc-PE commercial conjugate.

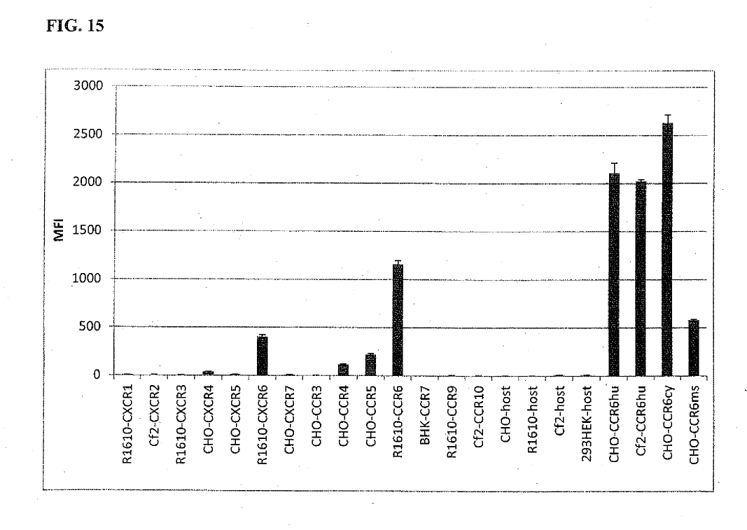

FIG. 15 depicts a graph of fluorescence of the cells as in FIG. 2 expressing CCR6 or other GPCRs, in the presence of human antibody IgG1 MSM-R621 of this invention and labeled with anti-human antibody Fc-PE commercial conjugate.

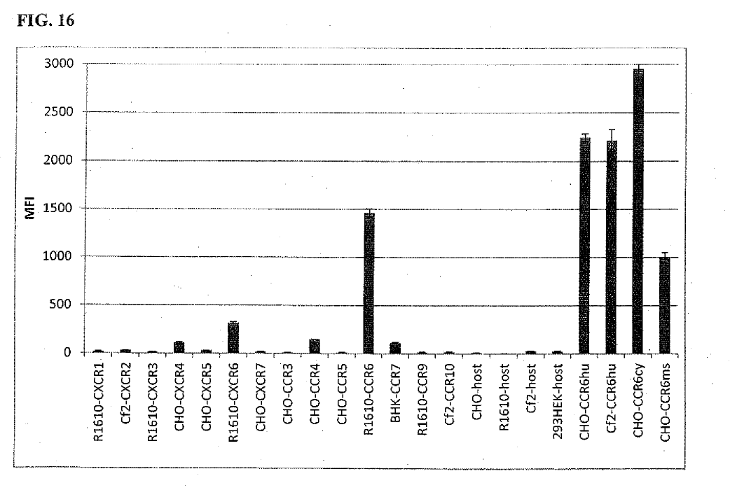

FIG. 16 depicts a graph of fluorescence of the cells as in FIG. 2 expressing CCR6 or other GPCRs, in the presence of human antibody IgG1 MSM-R622 of this invention and labeled with anti-human antibody Fc-PE commercial conjugate.

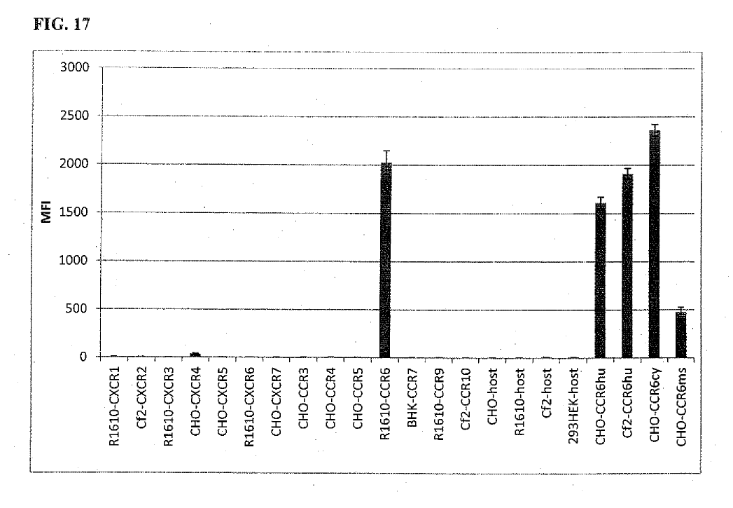

FIG. 17 depicts a graph of fluorescence of the cells as in FIG. 2 expressing CCR6 or other GPCRs, in the presence of human antibody IgG1 MSM-R623 of this invention and labeled with anti-human antibody Fc-PE commercial conjugate.

The data shown in FIGS. 2-17 demonstrated that all CCR6 IgG1 antibody clones selectively bind to human CCR6 and cyno CCR6, whereas no or only weak binding to other G-protein coupled receptors was observed. Several IgG1 antibodies of this invention also displayed binding to the mouse CCR6 ortholog, which can be advantageous for exploring the role of CCR6 in a broader range of mouse models of human diseases, as well as in validating novel animal models. Mouse and human CCR6 molecules are homologous only to a certain degree and the cross-reactivity was desired but not quite expected. The discovery of human-mouse cross-reactivity in some antibodies of this invention helps in the following animal based evaluation studies on the antibodies for selection of antibody candidates for therapeutics development. Because of this cross-reactivity, studies performed using murine systems are reasonably predictive of effects observed in human beings.

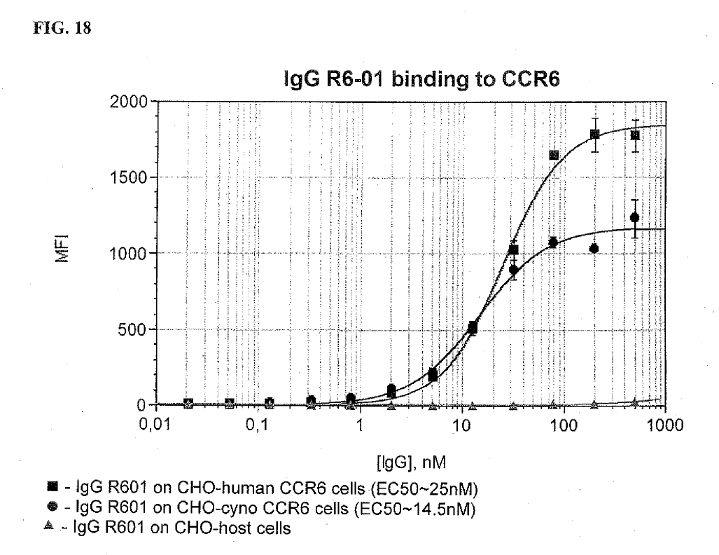

The affinity of IgG1 antibodies to human CCR6 or cyno CCR6 was evaluated by measuring mean fluorescence value (MFI) of cells expressing CCR6 in the presence of varying concentration of antibody in question, upon staining of bound to the cells antibodies with a commercial anti-human antibody-PE conjugate. The data of such experiments are depicted in FIGS. 18-33. In these experiments also binding of some antibodies to parental cells or cells expressing mouse CCR6 was also quantitatively characterized by obtaining EC₅₀ value (a concentration of IgG1 at which half-maximum MFI is achieved) for each curve using the SoftMaxPro5 program.

The EC₅₀ values for human CCR6 and cyno CCR6 binding of the antibodies of this invention were in nanomolar range. Thus, FIG. 18 depicts a graph of fluorescence of CHO cells expressing human CCR6, cynoCCR6, and CHO host cells in the presence of varying concentration of IgG1 antibodies of this invention MSM-R601 and labeled with a commercial anti-human Fc PE-conjugate.

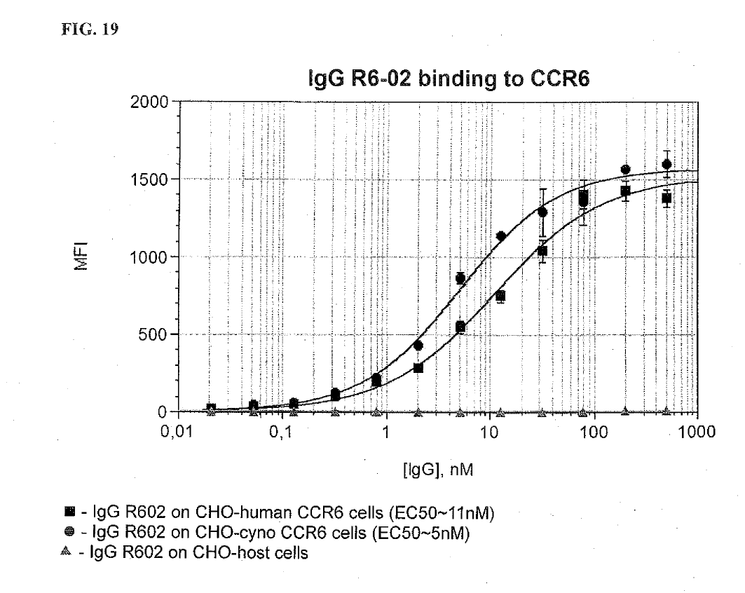

FIG. 19 depicts a graph of fluorescence of CHO cells expressing human CCR6, cynoCCR6, and CHO host cells in the presnce of varying concentration of IgG1 antibodies of this invention MSM-R602 and labeled with a commercial anti-human Fc PE-conjugate.

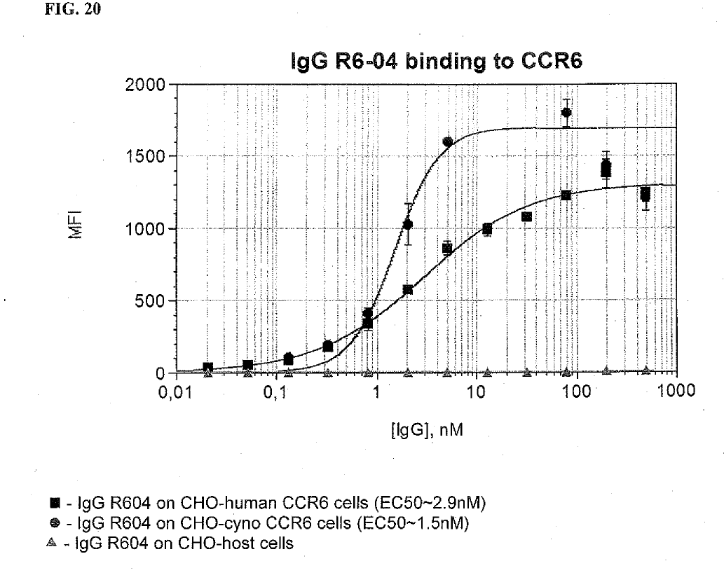

FIG. 20 depicts a graph of fluorescence of CHO cells expressing human CCR6, cynoCCR6, and CHO host cells in the presence of varying concentration of IgG1 antibodies of this invention MSM-R604 and labeled with a commercial anti-human Fc PE-conjugate.

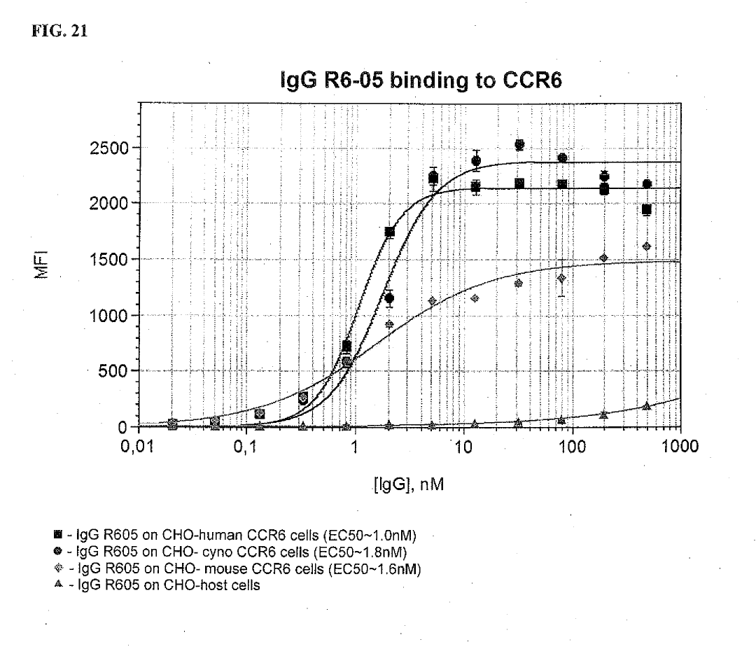

FIG. 21 depicts a graph of fluorescence of CHO cells expressing human CCR6, cynoCCR6, mouse CCR6 and CHO host cells in the presence of varying concentration of IgG1 antibodies of this invention MSM-R605 and labeled with a commercial anti-human Fc PE-conjugate.

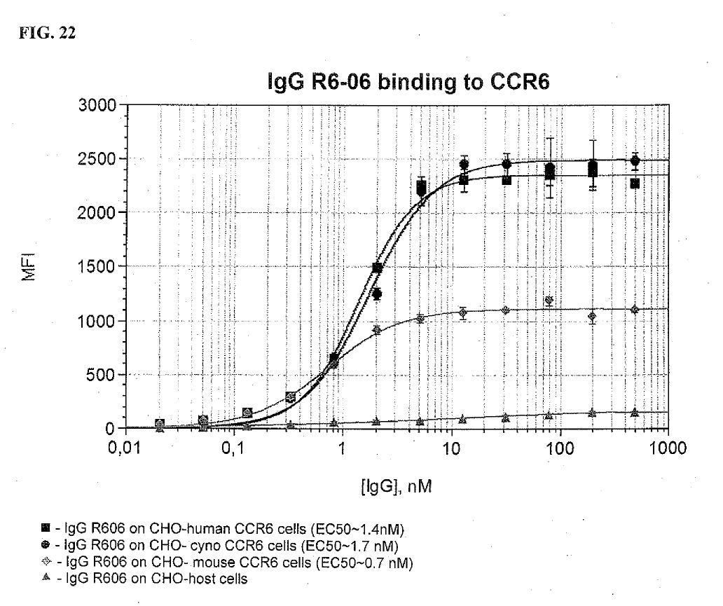

FIG. 22 depicts a graph of fluorescence of CHO cells expressing human CCR6, cynoCCR6, mouse CCR6 and CHO host cells in the presence of varying concentration of IgG1 antibodies of this invention MSM-R606 and labeled with a commercial anti-human Fc PE-conjugate.

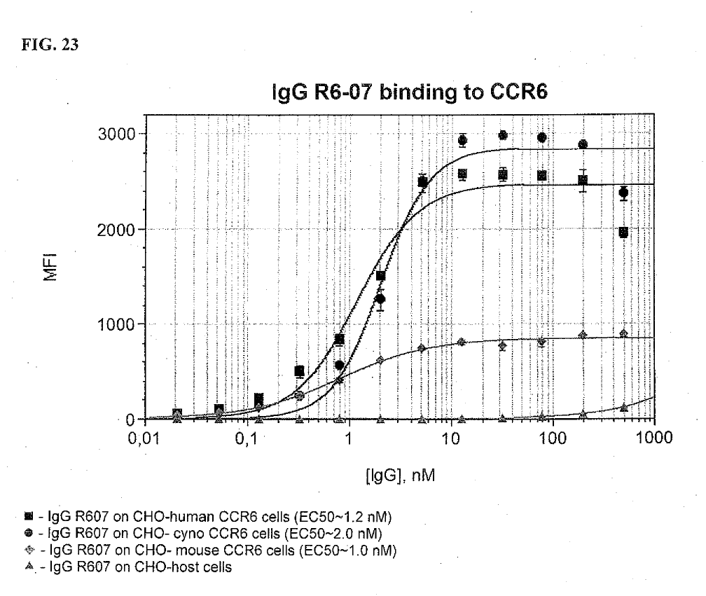

FIG. 23 depicts a graph of fluorescence of CHO cells expressing human CCR6, cynoCCR6, mouse CCR6 and CHO host cells in the presence of varying concentration of IgG1 antibodies of this invention MSM-R607 and labeled with a commercial anti-human Fc PE-conjugate.

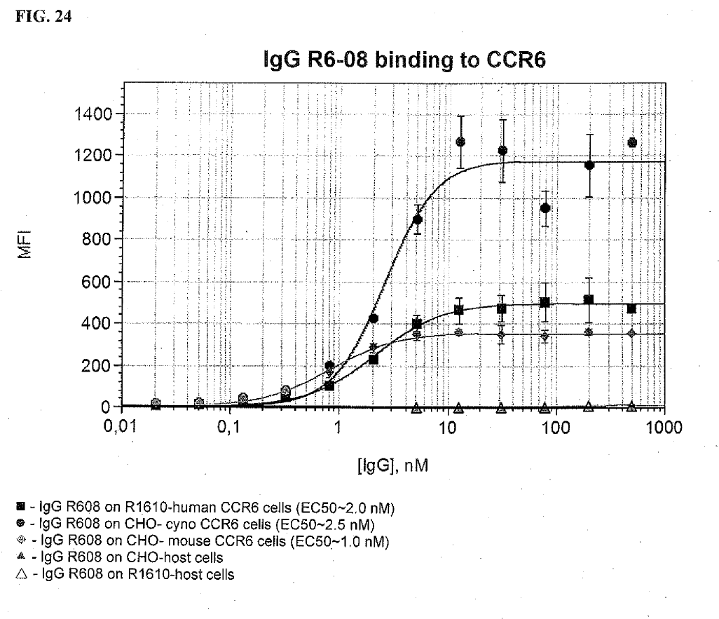

FIG. 24 depicts a graph of fluorescence of CHO cells expressing human CCR6, cynoCCR6, mouse CCR6 and CHO host cells in the presence of varying concentration of IgG antibodies of this invention MSM-R608 and labeled with a commercial anti-human Fc PE-conjugate.

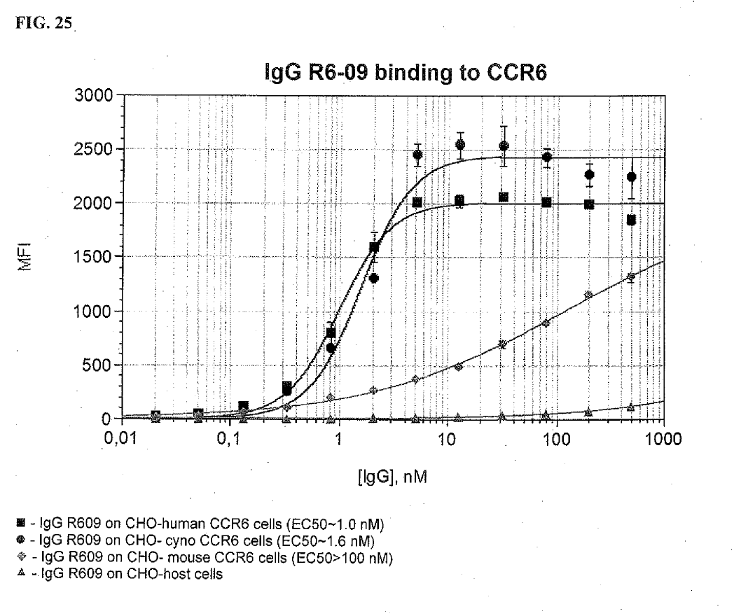

FIG. 25 depicts a graph of fluorescence of CHO cells expressing human CCR6, cynoCCR6, mouse CCR6 and CHO host cells in the presence of varying concentration of IgG1 antibodies of this invention MSM-R609 and labeled with a commercial anti-human Fc PE-conjugate.

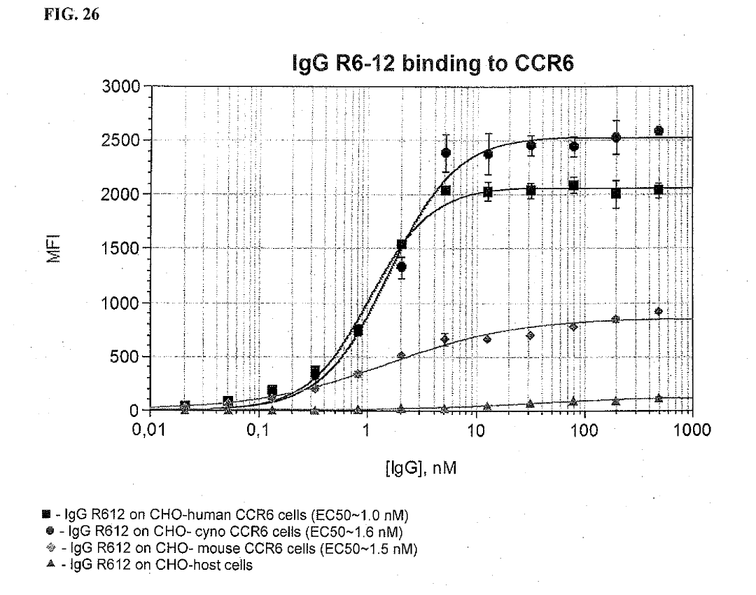

FIG. 26 depicts a graph of fluorescence of CHO cells expressing human CCR6, cynoCCR6, mouse CCR6 and CHO host cells in the presnce of varying concentration of IgG1 antibodies of this invention MSM-R612 and labeled with a commercial anti-human Fc PE-conjugate.

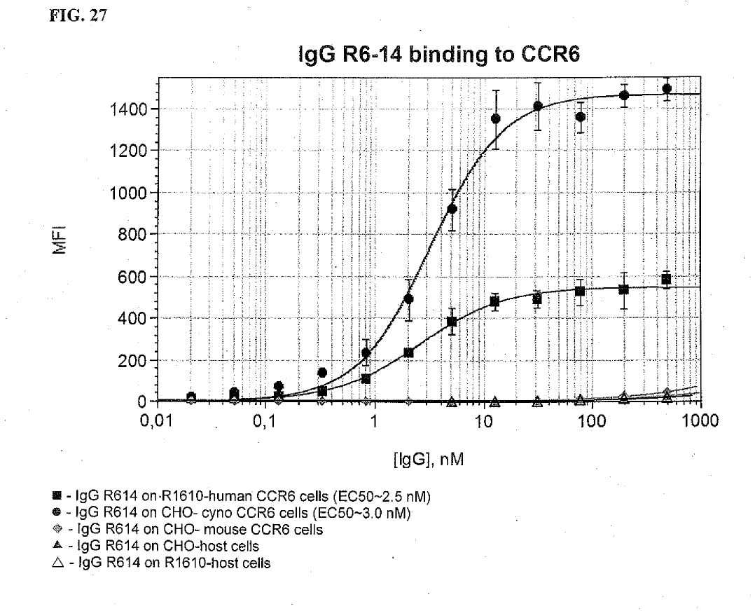

FIG. 27 depicts a graph of fluorescence of R1610 cells expressing human CCR6, and CHO cells expressing cynoCCR6, and mouse CCR6 and R1610 and CHO host cells in the presence of varying concentration of IgG1 antibodies of this invention MSM-R614 and labeled with a commercial anti-human Fc PE-conjugate.

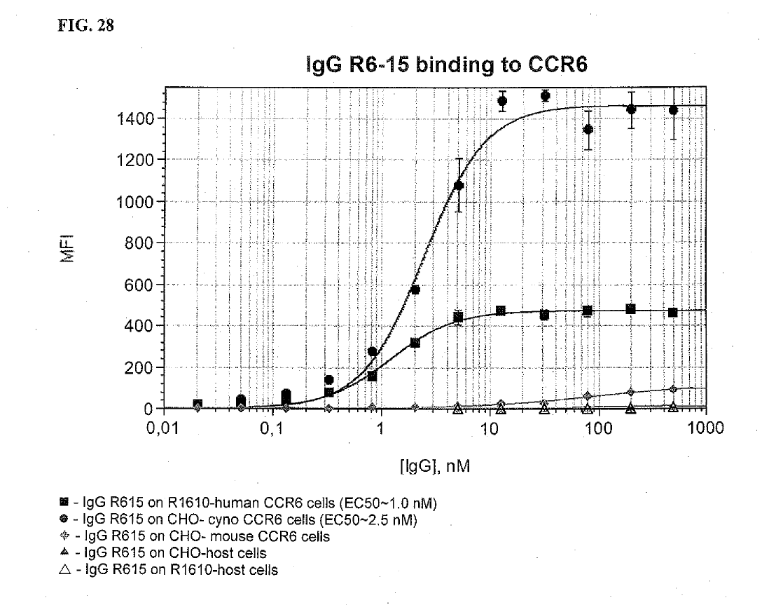

FIG. 28 depicts a graph of fluorescence of R1610 cells expressing human CCR6, and CHO cells expressing cynoCCR6, and mouse CCR6 and R1610 and CHO host cells in the presence of varying concentration of IgG1 antibodies of this invention MSM-R615 and labeled with a commercial anti-human Fc PE-conjugate.

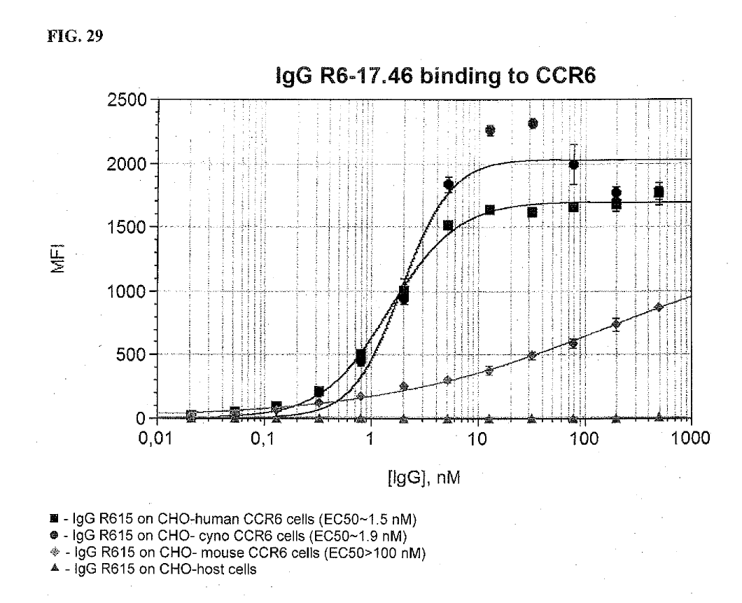

FIG. 29 depicts a graph of fluorescence of CHO cells expressing human CCR6, cynoCCR6, mouse CCR6 and CHO host cells in the presence of varying concentration of IgG1 antibodies of this invention MSM-R629 and labeled with a commercial anti-human Fc PE-conjugate.

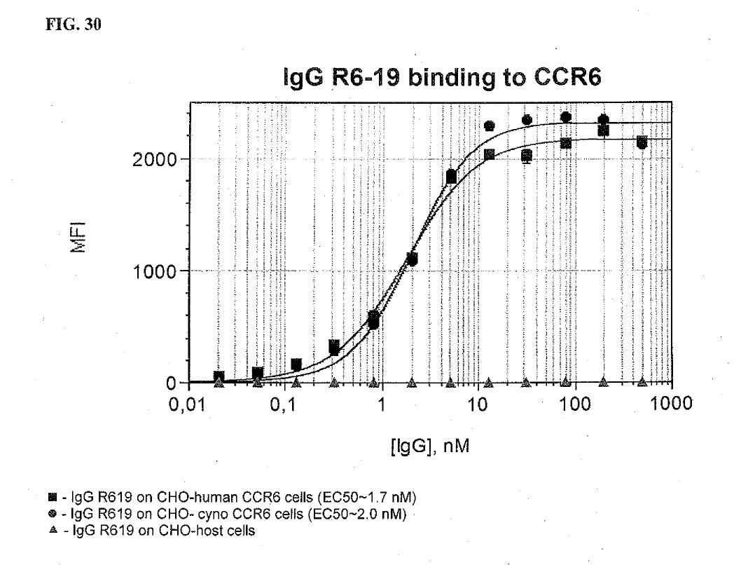

FIG. 30 depicts a graph of fluorescence of CHO cells expressing human CCR6, cynoCCR6 and CHO host cells in the presence of varying concentration of IgG1 antibodies of this invention MSM-R619 and labeled with a commercial anti-human Fc PE-conjugate.

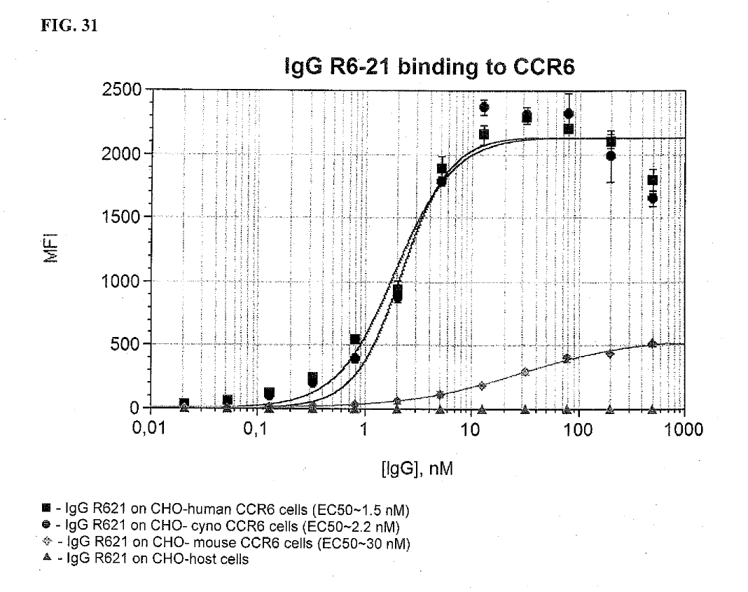

FIG. 31 depicts a graph of fluorescence of CHO cells expressing human CCR6, cynoCCR6, mouse CCR6 and CO host cells in the presence of varying concentration of IgG1 antibodies of this invention MSM-R621 and labeled with a commercial anti-human Fc PE-conjugate.

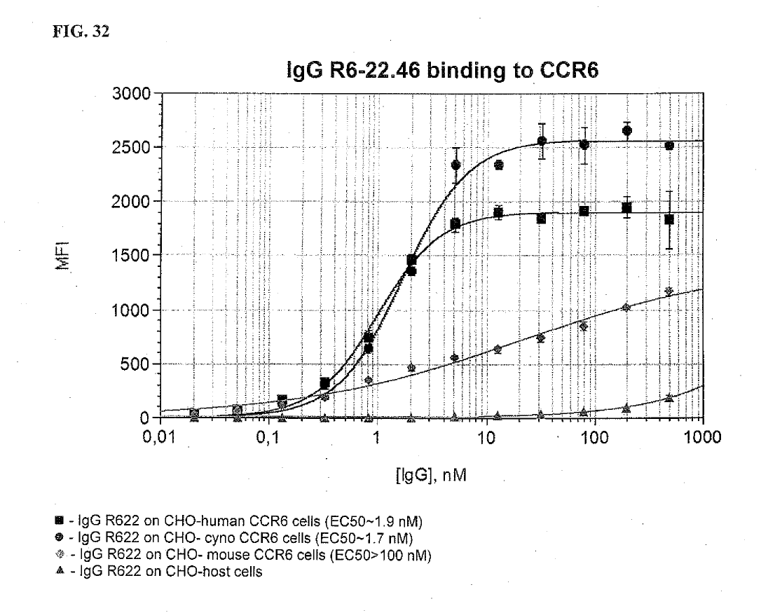

FIG. 32 depicts a graph of fluorescence of CHO cells expressing human CCR6, cynoCCR6, mouse. CCR6 and CHO host cells in the presence of varying concentration of IgG1 antibodies of this invention MSM-R622 and labeled with a commercial anti-human Fc PE-conjugate.

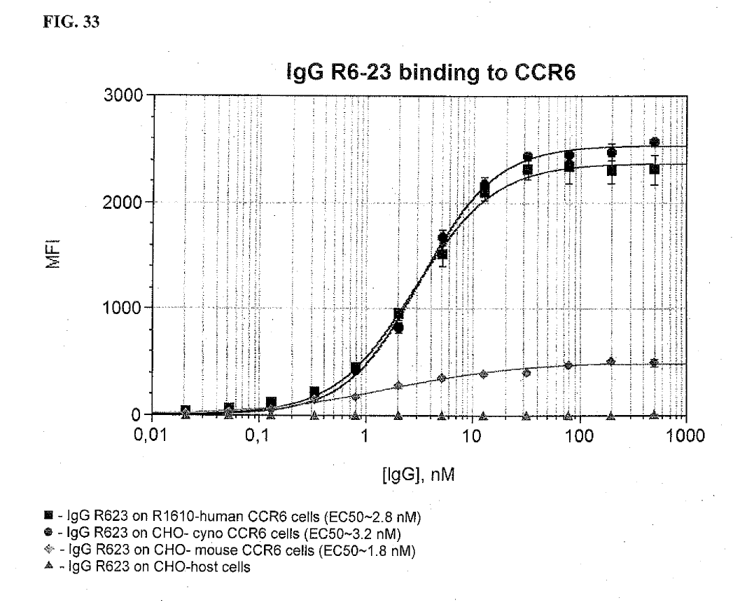

FIG. 33 depicts a graph of fluorescence of CHO cells expressing human CCR6, cynoCCR6, mouse CCR6 and CHO host cells in the presence of varying concentration of IgG1 antibodies of this invention MSM-R623 and labeled with a commercial anti-human Fc PE-conjugate.

The EC50 values for human CCR6 and its cyno and mouse orthologs obtained as presented in the FIGS. 18-33 are provided in the Table 2 below;

TABLE 2
EC₅₀ and IC₅₀ of Anti-CCR6 IgG1 Antibodies of This Invention

| MSM-Number | EC₅₀, nM | | | IC₅₀, nM | |
|---|---|---|---|---|---|
| IgG1 | CCR6 human | CCR6 cyno | CCR6 mouse | CCL20-Ca-Flux | Specificity |
| R601 | 25 | 14.5 | n/b* | n/f* | OK |
| R602 | 11 | 5 | n/b | n/f | OK |
| R604 | 2.9 | 1.5 | n/b | n/f | OK |
| R605 | 1.0 | 1.8 | 1.6 | 1.5 | |
| R606 | 1.4 | 1.7 | 0.7 | 8.3 | |
| R607 | 1.2 | 2.0 | 1.0 | 13.5 | Cross CXCR6 |
| R608 | 2.0 | 2.5 | 1.0 | n/f | |
| R609 | 1.0 | 1.6 | >100 | n/f | |
| R610 | >100 | >100 | n/b | n/f | |
| R612 | 1.0 | 1.6 | 1.5 | n/f | |
| R614 | 2.5 | 3.0 | n/b | n/f | |
| R615 | 1.0 | 2.5 | n/b | n/f | |
| R617 | 1.5 | 1.9 | >100 | n/f | OK |
| R619 | 1.7 | 2.0 | n/b | n/f | OK |
| R621 | 1.5 | 2.2 | 30 | n/f | Cross CXCR6 |
| R622 | 1.9 | 1.7 | >100 | n/f | Cross CXCR6 |
| R623 | 2.8 | 3.2 | 1.8 | n/f | OK |

*n/b - non-binder;
**n/f - non-functional

### Example 7

### Anti-CCR6 Antibodies Inhibit Signaling of the Natural Ligand to CCR6

To determine if fully human, antibodies against CCR6 can be useful in inhibiting CCR6 certain mediated disorder in which signaling via CCR6-CCL20 axis can be desirable to block, we performed experiments to determine whether such antibodies can inhibit signaling by naturally occurring CCR6 ligand CCL20.

Upon binding of a natural ligand human CCL20 to human CCR6 on cell surface, the cell typically respond by transiently increasing intracellular concentration of calcium cations, known as Ca-flux. The Ca-flax was fluorescence measured in commercial Chem-1 cells expressing CCR6 (EMD Millipore) pre-loaded with a Ca-sensitive fluorescent dye using protocol and Calcium 5 Assay Kit of Molecular Devices (Catalog number R8185), upon addition of CCL20 to final concentration 30 nM, respectively. The IC₅₀ value corresponding to the concentration of an antibody at which half inhibition of Ca-Flux occurs was determined for each curve using the SoftMaxPro5 program; the values are provided in Table 2 above.

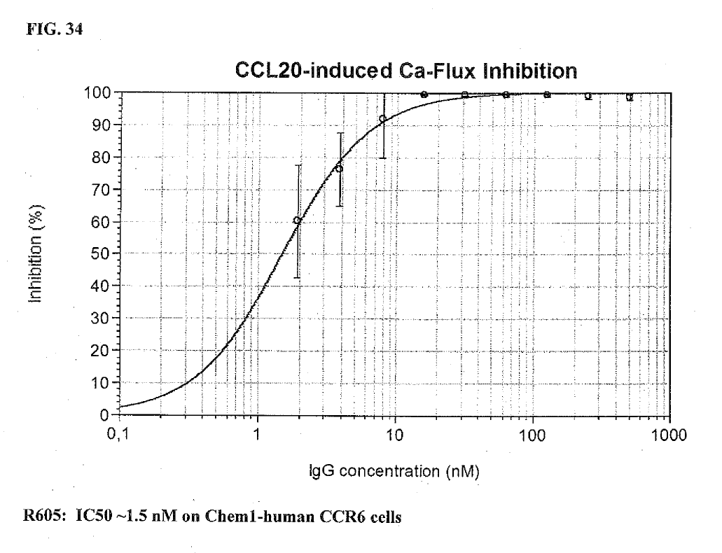

FIG. 34 depicts a graph of inhibition of the increase in the intracellular Ca concentration in response to addition of human CCL20 to Chem-1 cells expressing human CCR6 in the presence of inhibiting IgG1 antibodies MSM-R605 at varying concentration.

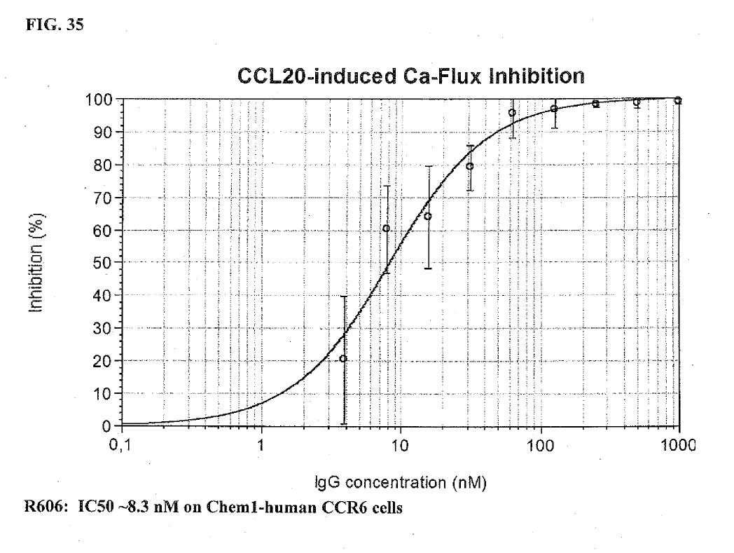

FIG. 35 depicts a graph of inhibition of the increase in the intracellular Ca concentration in response to addition of human CCL20 to Chem-1 cells expressing human CCR6 in the presence of inhibiting IgG1 antibodies MSM-R606 at varying concentration.

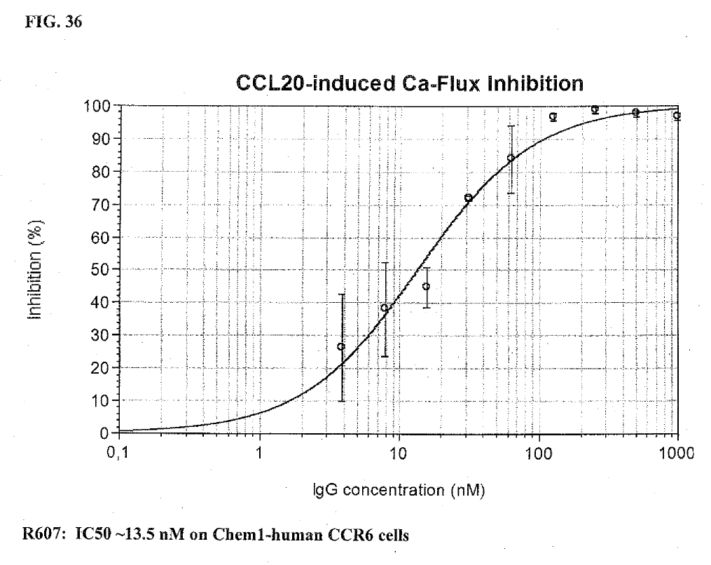

FIG. 36 depicts a graph of inhibition of the increase in the intracellular Ca concentration in response to addition of human CCL20 to Chem-1 cells expressing human CCR6 in the presence of inhibiting IgG1 antibodies MSM-R607 at varying concentration.

We conclude from these studies that these three antibodies, IgG1 MSM-R605, MSM-R606, and MSM-R607 inhibited signaling by natural ligand for CCR6 with IC₅₀s in nanomolar range. Without being bound by any particular theory of operation, we believe that the antibodies of this invention bound to natively configured CCR6 in such a way as to at least partially cover the binding site for the CCR6 ligand, therefore decreasing binding of the ligand for the binding site of CCR6. Regardless of the theory, we conclude that fully human antibodies of this invention can be used to decrease the effects of over-stimulation of CCR6 in a variety of conditions and disorders, including cancers.

### Example 8

### Sequencing of Antibody Fragments

All 17 fully human antibodies characterized as described above and summarized in Table 2 were sequenced. In addition, we disclose sequences of two more antibodies that were discovered as described above, purified in IgG1 format, and shown to bind cells expressing human CCR6 and cyno CCR6 but not parental cells. All these sequences are provided in Tables 3 through 30. Each of the 28 clones has a unique sequence and thus Tables 3 through 30 do not include duplications. For each antibody in Tables 2 and 3-20 sequences of heavy chain variable domain in DNA format (VH DNA) and of light chain in DNA format (VL DNA), the same sequences in amino acid format (HC AA and LC AA, respectively), as well as amino acid sequences of 1^{st}, 2^{nd}, and 3d CDRs of heavy chain (CDR1 HC AA, CDR2 HC AA, CDR3 BC AA) and of light chain (CDR1 LC AA, CDR2 LC AA, CDR3 LC AA). For the antibodies disclosed in Tables 21-30, only the amino acid sequences for heavy chain (HC), light chain (LC), and CDRs1-3 for heavy chain and light chain.

TABLE 3
Sequences of Anti-CCR6 Antibody MSM R601

| Sequence | | |
|---|---|---|
| Id No: | Sequence of | Sequence |
| SEQ ID NO: 1 | VH DNA | GAAGTTCAACTGCTGGAGTCCGGTGGTGGTCTGGTACAGCCG |
| | | GGTGGTTCTCTGCGTCTGAGTTGCGCGGCCAGTGGCTTTACCT |
| | | TCAGTAGCTATGCCATGAGCTGGGTGCGTCAGGCGCCGGGCA |
| | | AAGGTCTGGAATGGGTTAGCGCGATTAGCGGTAGTGGCGGTA |
| | | GCACGTACTATGCGGATAGCGTGAAAGGCCGTTTTACCATTTC |
| | | TCGCGACAACAGCAAGAACACGCTGTACCTGCAGATGAACTC |
| | | ACTGCGTGCCGAAGATACGGCCATGTATTACTGTGCGAGACA |
| | | TGGCTACTCCGCTACAAGGCAGGGACTCGACTACTGGGGCCA |
| | | GGGAACCTTGGTCACCGTCTCG |
| SEQ ID NO: 2 | VL DNA | GAAATTGTGCTGACCCAGTCTCCGGGCACGTTATCTCTGAGCC |
| | | CTGGTGAGCGCGCCACTCTGTCATGCCGGGCTTCTCAAAGTGT |
| | | TAGCAGTAGCTACCTGGCGTGGTATCAGCAAAAACCGGGCCA |
| | | GGCCCCGCGTCTGCTGATTTACGGTGCATCCAGCCGTGCCACC |
| | | GGCATTCCAGATGGTTTTTGCGGTAGTGGTTCTGGGACGGACT |
| | | TCACTCTGACAATCTCACGCCTGGAACCGGAGGATTTTGCGGT |
| | | GTATTACTGCCAGCAATATTCTTCTTCTCCTCTCACGTTCGGCC |
| | | AAGGGACCAAGGTGGAAATCAAA |
| SEQ ID NO: 3 | HC AA | EVQLLESGGGLVQPGGSLRLSCAASGFTFSSYAMSWVRQAPGKG |
| | | LEWVSAISGSGGSTYYADSVKGRFTISRDNSKNTLYLQMNSLRA |
| | | EDTAMYYCARHGYSATRQGLDYWGQGTLVTVSSASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKRVE |
| | | PKSCDKTHTCPPCPAPELLGGPSVFLFPPKPKDTLMISRTPEVTCV |
| | | VVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSREEMTKNQVSLTCLVKGFYPSDIAVEWESNGQPENNYK |
| | | TTPPVLDSDGSFFLYSKLTVDKSRWQQGNVFSCSVMHEALHNH |
| | | YTQKSLSLSPGK |
| SEQ ID NO: 4 | LC AA | EIVLTQSPGTLSLSPGERATLSCRASQSVSSSYLAWYQQKPGQAP |
| | | RLLIYGASSRATGIPDRFSGSGSGTDFTLTISRLEPEDFAVYYCQQ |
| | | YSSSPLTFGQGTKVEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYACEVTHQGLSSPVTKSFNRGEC |
| SEQ ID NO: 5 | CDR1 HC AA | FTFSSYAMSWVR |
| SEQ ID NO: 6 | CDR2 HC AA | VSAISGSGGSTYYADS |
| SEQ ID NO: 7 | CDR3 HC AA | CARHGYSATRQGLDYWGQ |
| SEQ ID NO: 8 | CDR1 LC AA | RASQSVSSSYLA |
| SEQ ID NO: 9 | CDR2 LC AA | GASSRAT |
| SEQ ID NO: | CDR3 LC AA | CQQYSSSPLTFGQ |
| 10 | | |

TABLE 4
Sequences of Anti-CCR6 Antibody MSM R602

| Sequence | | |
|---|---|---|
| Id No: | Sequence of: | Sequence |
| SEQ ID NO: 11 | VH DNA | GAAGTTCAACTGCTGGAGTCCGGTGGTGGTCTGGTACAG |
| | | CCGGGTGGTTCTCTGCGTCTGAGTTGCGCGGCCAGTGGC |
| | | TTTACCTTCAGTAGCTATGCCATGAGCTGGGTGCGTCAG |
| | | GCGCCGGGCAAAGGTCTGGAATGGGTTAGCGCGATTAG |
| | | CGGTAGTGGCGGTAGCACGTACTATGCGGATAGCGTGA |
| | | AAGGCCGTTTTACCATTTCTCGCGACAACAGCAAGAACA |
| | | CGCTGTACCTGCAGATGAACTCACTGCGTGCCGAAGATA |
| | | CGGCCGTGTATTACTGTGCGAGAAAAGGCTATTACGGAG |
| | | GTATGGCAATGGACTACTGGGGCCAGGGAACCTTGGTC |
| | | ACCGTCTCG |
| SEQ ID NO: 12 | VL DNA | GAAATTGTGCTGACCCAGTCTCCGGGCACGTTATCTCTG |
| | | AGCCCTGGTGAGCGCGCCACTCTGTCATGCCGGGCTTCT |
| | | CAAAGTGTTAGCAGTAGCTACCTGGCGTGGTATCAGCAA |
| | | AAACCGGGCCAGGCCCCGCGTCTGCTGATTTACGGTGCA |
| | | TCCAGCCGTGCCACCGGCATTCCAGATCGTTTTTCCGGT |
| | | AGTGGTTCTGGGACGGACTTCACTCTGACAATCTCACGC |
| | | CTGGAACCGGAGGATTTTGCCGTGTATTACTGCCAGCAA |
| | | TATTCTTCTTCTCCTCTCACGTTCGGCCAAGGGACCAAG |
| | | GTGGAAATCAAA |
| SEQ ID NO: 13 | HC AA | EVQLLESGGGLVQPGGSLRLSCAASGFTFSSYAMSWVRQA |
| | | PGKGLEWVSAISGSGGSTYYADSVKGRFTISRDNSKNTLYL |
| | | QMNSLRAEDTAVYYCARKGYYGGMAMDYWGQGTLVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTY |
| | | ICNVNHKPSNTKVDKRVEPKSCDKTHTCPPCPAPELLGGPS |
| | | VFLFPPKPKDTLMISRTPEVTCVVVDVSHEDPEVKFNWYV |
| | | DGVEVHNAKTKPREEQYNSTYRVVSVLTVLHQDWLNGKE |
| | | YKCKVSNKALPAPIEKTISKAKGQPREPQVYTLPPSREEMT |
| | | KNQVSLTCLVKGFYPSDIAVEWESNGQPENNYKTTPPVLD |
| | | SDGSFFLYSKLTVDKSRWQQGNVFSCSVMHEALHNHYTQ |
| | | KSLSLSPGK |
| SEQ ID NO: 14 | LC AA | EIVLTQSPGTLSLSPGERATLSCRASQSVSSSYLAWYQQKP |
| | | GQAPRLLIYGASSRATGIPDRFSGSGSGTDFTLTISRLEPEDF |
| | | AVYYCQQYSSSPLTFGQGTKVEIKRTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSP |
| | | VTKSFNRGEC |
| SEQ ID NO: 15 | CDR1 HC AA | FTFSSYAMSWVR |
| SEQ ID NO: 16 | CDR2 HC AA | VSAISGSGGSTYYADS |
| SEQ ID NO: 17 | CDR3 HC AA | CARKGYYGGMAMDYWGQ |
| SEQ ID NO: 18 | CDR1 LC AA | RASQSVSSSYLA |
| SEQ ID NO: 19 | CDR2 LC AA | GASSRAT |
| SEQ ID NO: 20 | CDR3 LC AA | CQQYSSSPLTFGQ |

TABLE 5
Sequences of Anti-CCR6 Antibod MSM R604

| Sequence | | |
|---|---|---|
| Id No: | Sequence of: | Sequence |
| SEQ ID NO: 21 | VH DNA | GAAGTTCAACTGCTGGAGTCCGGTGGTGGTCTGGTAC |
| | | AGCCGGGTGGTTCTCTGCGTCTGAGTTGTGCGGCCAG |
| | | TGGCTTTACCTTCACTAACTATTCTATGCATTGGGTGC |
| | | GTCAGGCTCCGGGCAAAGGTCTGGAATGGGTTAGCTT |
| | | GATTAGTCCGTATAGTAGCTCGACCTACTATGCTGAT |
| | | AGCATGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGAGGT |
| | | CGTTACTACGGTGCTTACTCTTACCCGGGCTTCGACTA |
| | | CTGGGGCCAGGGAACCTTGGTCACCGTCTCG |
| SEQ ID NO: 22 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATCGGCTAAGAATCCTCTCACGTTC |
| | | GGCCAAGGGACCAAGGTGGAAATCAAA |
| SEQ ID NO: 23 | HC AA | EVQLLESGGGLVQPGGSLRLSCAASGFTFTNYSMHWVR |
| | | QAPGKGLEWVSLISPYSSSTYYADSMKGRFTISRDNSKN |
| | | TLYLQMNSLRAEDTAVYYCARGRYYGAYSYPGFDYWG |
| | | QGTLVTVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIAV |
| | | EWESNGQPENNYKTTPPVLDSDGSFFLYSKLTVDKSRW |
| | | QQGNVFSCSVNMHEALHNHYTQKSLSLSPGK |
| SEQ ID NO: 24 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQSAKNPLTFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 25 | CDR1 HC AA | FTFTNYSMEWVR |
| SEQ ID NO: 26 | CDR2 HC AA | VSLISPYSSSTYYADS |
| SEQ ID NO: 27 | CDR3 HC AA | CARGRYYGAYSYPGFDYWGQ |
| SEQ ID NO: 28 | CDRI LC AA | RASQSVSSSYLA |
| SEQ ID NO: 29 | CDR2 LC AA | GASSRAT |
| SEQ ID NO: 30 | CDR3 LC AA | CQQSAKNPLTFGQ |

TABLE 6
Sequences of Anti-CCR6 Antibody MSM R605

| Sequence | | |
|---|---|---|
| Id No: | Sequence of: | Sequence |
| SEQ ID NO: 31 | VH DNA | GAAGTTCAACTGCTGGAGTCCGGTGGTGGTCTGGTAC |
| | | AGCCGGGTGGTTCTCTGCGTCTGAGTTGCGCGGCCAG |
| | | TGGCTTTACCTTCAGTAGCTATGCCATGAGCTGGGTG |
| | | CGTCAGGCGCCGGGCAAAGGTCTGGAATGGGTTAGC |
| | | GCGATTAGCGGTAGTGGCGGTAGCACGTACTATGCGG |
| | | ATAGCGTGAAAGGCCGTTTTACCATTTCTCGCGACAA |
| | | CAGCAAGAACACGCTGTACCTCTCAGATGAACTCACTG |
| | | CGTGCCGAAGATACGGCCGTGTATTACTGTGCGAGAA |
| | | ACTCTATGAAAGGTCAGCGTAAACAGCGTGGCATGGA |
| | | CTACTGGGGCCAGGGAACCTTGGTCACCGTCTCG |
| SEQ ID NO: 32 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATCTAGGGCGACTCCTCTCACGTTC |
| | | GGCCAAGGGACCAAGGTGGAAATCAAA |
| SEQ ID NO: 33 | HC AA | EVQLLESGGGLVQPGGSLRLSCAASGFTFSSYAMSWVR |
| | | QAPGKGLEWVSAISGSGGSTYYADSVKGRFTISRDNSKN |
| | | TLYLQMNSLRAEDTAVYYCARNSMKGQRKQRGMDYW |
| | | GQGTLVTVSSASTKGPSVFPLAPSSKSTSGGTAALGCLV |
| | | KDYFPEPVTVSWNSGALTSGVHTFPAVLQSSGLYSLSSV |
| | | VTVPSSSLGTQTYICNVNHKPSNTKVDKRVEPKSCDKTH |
| | | TCPPCPAPELLGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 34 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQSRATPLTFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 35 | CDR1 HC AA | FTFSSYAMSWVR |
| SEQ ID NO: 36 | CDR2 HC AA | VSAISGSGGSTYYADS |
| SEQ ID NO: 37 | CDR3 HC AA | CARNSMKGQRKQRGMDYWGQ |
| SEQ ID NO: 38 | CDR1 LC AA | RASQSVSSSYLA |
| SEQ ID NO: 39 | CDR2 LC AA | GASSRAT |
| SEQ ID NO: 40 | CDR3 LC AA | CQQSRATPLTFGQ |

TABLE 7
Sequences of Anti-CCR6 Antibody MSM R606

| Sequence | | |
|---|---|---|
| Id No: | Sequence of: | Sequence |
| SEQ ID NO: 41 | VH DNA | GAAGTTCAACTGCTGGAGTCCGGTGGTGGTCTGGTAC |
| | | AGCCGGGTGGTTCTCTGCGTCTGAGTTGTGCGGCCAG |
| | | TGGCTTTACCTTCAGTAACTATTATATCCATTGGGTGC |
| | | GTCAGGCTCCAGGCAAAGGTCTGGAATGGGTTAGCAT |
| | | TATTAATCCGTGGAGTGGCAGGACCTACTATGCGGAT |
| | | AGCGTGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGAGGC |
| | | AGGTTGCGGAGCCTCTCTTATTCCGGCATTGACTACTG |
| | | GGGCCAGGGAACCTTGGTCACCGTCTCG |
| SEQ ID NO: 42 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACCTGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATCTTCTTATTCTCCTATCACGTTCG |
| | | GCCAAGGGACCAAGGTGGAAATCAAA |
| SEQ ID NO: 43 | HC AA | EVQLLESGGGLVQPGGSLRLSCAASGFTFSNYYIHWVR |
| | | QAPGKGLEWVSIINPWSGRTYYADSVKGRFTISRDNSKN |
| | | TLYLQMNSLRAEDTAVYYCARGRLRSLSYSGIDYWGQ |
| | | GTLVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKRVEPKSCDKTHTC |
| | | PPCPAPELLGGPSVFLFPPKPKDTLMISRTPEVTCVVVDV |
| | | SHEDPEVKFNWYVDGVEVHNAKTKPREEQYNSTYRVV |
| | | SYLTVLHQDWLNGKEYKCKVSNKALPAPIEKTISKAKG |
| | | QPREPQVYTLPPSREEMTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGK |
| SEQ ID NO: 44 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQSSYSPITFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 45 | CDR1 HC AA | FTFSNYYIHWVR |
| SEQ ID NO: 46 | CDR2 HC AA | VSIINPWSGRTYYADS |
| SEQ ID NO: 47 | CDR3 HC AA | CARGRLRSLSYSGIDYWGQ |
| SEQ ID NO: 48 | CDR1 LC AA | RASQSVSSSYLA |
| SEQ ID NO: 49 | CDR2 LC AA | GASSRAT |
| SEQ ID NO: 50 | CDR3 LC AA | CQQSSYSPITFGQ |

TABLE 8
Sequences of Anti-CCR6 Antibody MSM R607

| Sequence | | |
|---|---|---|
| Id No: | Sequence of | Sequence |
| SEQ ID NO: 51 | VH DNA | GAAGTTCAACTGCTGGAGTCCGGTGGTGGTCTGGTAC |
| | | AGCCGGGTGGTTCTCTGCGTCTGAGTTGTGCGGCCAG |
| | | TGGCTTTACCTTCAGTTCGTATTATATGCATTGGGTGC |
| | | GTCAGCTCTCGGGGCAAAGGTCTGGAATGGGTTAGCTT |
| | | GATTGCTCCGGCGAATAGCGATACCTACTATGCGGAT |
| | | AGCGTGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGATCT |
| | | TCTTACACTCGTCATTCTTCTAAACGTGGCATGGACTA |
| | | CTGGGGCCAGGGAACCTTGGTCACCGTCTCG |
| SEQ ID NO: 52 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAAGCTGCTTATTCTCCTATCACGTTCG |
| | | GCCAAGGGACCAAGGTGGAAATCAAA |
| SEQ ID NO: 53 | HC AA | EVQLLESGGGLVQPGGSLRLSCAASGFTFSSYYMHWVR |
| | | QAPGKGLEWVSLIAPANSDTYYADSVKGRFTISRDNSK |
| | | NTLYLQMNSLRAEDTAVYYCARSSYTRHSSKRGMDYW |
| | | GQGTLVTVSSASTKGPSVFPLAPSSKSTSGGTAALGCLV |
| | | KDYFPEPVTVSWNSGALTSGVHTFPAVLQSSGLYSLSSV |
| | | VTVPSSSLGTQTYICNVNHKPSNTKVDKRVEPKSCDKTH |
| | | TCPPCPAPELLGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 54 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQAAYSPITFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 55 | CDR1 HC AA | FTFSSYYMHWVR |
| SEQ ID NO: 56 | CDR2 HC AA | VSLIAPANSDTYYADS |
| SEQ ID NO: 57 | CDR3 HC AA | CARSSYTRHSSKRGMDYWGQ |
| SEQ ID NO: 58 | CDR1 LC AA | RASQSVSSSYLA |
| SEQ ID NO: 59 | CDR2 LC AA | GASSRAT |
| SEQ ID NO: 60 | CDR3 LC AA | CQQAAYSPITFGQ |

TABLE 9
Sequences of Anti-CCR6 Antibody MSM R608

| Sequence | | |
|---|---|---|
| Id No: | Sequence of: | Sequence |
| SEQ ID NO: 61 | VH DNA | GAAGTGCAACTGGTACAGTCCGGTGCGGAAGTTAAA |
| | | AAGCCGGGTTCTAGTGTCAAGGTGTCATGCAAAGCCA |
| | | GTGGCGGCACCCTCACTTCTTATTATATCTCTTGGGTG |
| | | CGTCAGGCCCCGGGCCAAGGTCTGGAATGGATGGGTT |
| | | ATATTTCTCCGTCTTCTGGCTATACCTATTATGCGCAG |
| | | AAATTTCAAGGCCGTGTGACCATTACTGCTGATGAAA |
| | | GCACTTCTACAGCCTACATGGAGCTGAGTTCACTGCG |
| | | CTCCGAGGACACGGCCGTGTATTACTGTGCGAGACGT |
| | | TACCGTGGTCGTGGTTACGTTTACGGCTTGGACTACTG |
| | | GGGCCAGGGAACCCTGGTCACCGTCTCG |
| SEQ ID NO: 62 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATCTTATTATGCTCCTGTCACGTTCG |
| | | GCCAAGGGACCAAGGTGGAAATCAAA |
| SEQ ID NO: 63 | HC AA | EVQLVQSGAEVKKPGSSVKVSCKASGGTLTSYYISWVR |
| | | QAPGQGLEWMGYISPSSGYTYYAQKFQGRVTITADEST |
| | | STAYMELSSLRSEDTAVYYCARRYRGRGYVYGLDYWG |
| | | QGTLVTVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIAV |
| | | EWESNGQPENNYKTTPPVLDSDGSFFLYSKLTVDKSRW |
| | | QQGNVFSCSVMHEALHNHYTQKSLSLSPGK |
| SEQ ID NO: 64 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQSYYAPVTFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 65 | CDR1 HC AA | GTLTSYYISWVR |
| SEQ ID NO: 66 | CDR2 HC AA | MGYISPSSGYTYYAQK |
| SEQ ID NO: 67 | CDR3 HC AA | CARRYRGRGYVYGLDYWGQ |
| SEQ ID NO: 68 | CDR1 LC AA | RASQSVSSSYLA |
| SEQ ID NO: 69 | CDR2 LC AA | GASSRAT |
| SEQ ID NO: 70 | CDR3 LC AA | CQQSYYAPVTFGQ |

TABLE 10
Sequences of Anti-CCR6 Antibody MSM R609

| Sequence | | |
|---|---|---|
| Id No: | Sequence of: | Sequence |
| SEQ ID NO: 71 | VH DNA | GAAGTGCAACTGCTGGAGTCCGGTGGTGGTCTGGTAC |
| | | AGCCGGGTGGTTCTCTGCGTCTGAGTTGTGCGGCCAG |
| | | TGGCTTTACCTTCACTGCATCTTATATGAGCTGGGTGC |
| | | GTCAGGCTCCGGGCAAAGGTCTGGAATGGGTTAGCAA |
| | | TATTTCTCCGAAGTATGGCAATACCTACTATGCGGAT |
| | | AGCGTGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGAAGC |
| | | ATCAGGAGCGCCAAGTATTCCTATTACGGGATTGACT |
| | | ACTGGGGCCAGGGAACCCTGGTCACCGTCTCG |
| SEQ ID NO: 72 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTALGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATCTGCTTCTGCTCCTGTCACGTTCG |
| | | GCCAAGGGACCAAGGTGGAAATCAAA |
| SEQ ID NO: 73 | HC AA | EVQLLESGGGLVQPGGSLRLSCAASGFTFTASYMSWVR |
| | | QAPGKGLEWVSNISPKYGNTYYADSVKGRFTISRDNSK |
| | | NTLYLQMNSLRAEDTAVYYCARSIRSAKYSYYGIDYWG |
| | | QGTLVTVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIAV |
| | | EWESNGQPENNYKTTPPVLDSDGSFFLYSKLTVDKSRW |
| | | QQGNVFSCSVMHEALHNHYTQKSLSLSPGK |
| SEQ ID NO: 74 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQSASAPVTFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 75 | CDR1 HC AA | FTFTASYMSWVR |
| SEQ ID NO: 76 | CDR2 HC AA | VSNISPKYGNTYYADS |
| SEQ ID NO: 77 | CDR3 HC AA | CARSIRSAKYSYYGIDYWGQ |
| SEQ ID NO: 78 | CDR1 LC AA | RASQSVSSSYLA |
| SEQ ID NO: 79 | CDR2 LC AA | GASSRAT |
| SEQ ID NO: 80 | CDR3 LC AA | CQQSASAPVTFGQ |

TABLE 11
Sequences of Anti-CCR6 Antibody MSM R610

| Sequence | | |
|---|---|---|
| Id No: | Sequence of: | Sequence |
| SEQ ID NO: 81 | VH DNA | GAAGTTCAACTGCTGGAGTCCGGTGGTGGTCTGGTAC |
| | | AGCCGGGTGGTTCTCTGCGTCTGAGTTGCGCGGCCAG |
| | | TGGCTTTACCTTCACTGGCTCTTCTATGAGCTGGGTGC |
| | | GTCAGGCTCCGGGCAAAGGTCTGGAATGGGTTAGCCT |
| | | TATTTCGTCGAGGGGTAGCTATACCTACTATGCGGAT |
| | | AGCGTGAAAGGCCGTTTTACCATTTCTCGGGACAACA |
| | | GCAAGAACACACTGTACCTGCAGATGAACTCACTGCG |
| | | TGCCGAAGATACGGCCGTGTATTACTGTGCGAGATCT |
| | | TACGCTTACCAGTACCGTGGCTTGGACTACTGGGGCC |
| | | AGGGAACCTTGGTCACCGTCTCG |
| SEQ ID NO: 82 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAAAATAGGACGTCTCCTATCACGTTC |
| | | GGCCAAGGGACCAAGGTGGAAATCAAA |
| SEQ ID NO: 83 | HC AA | EVQLLESGGGLVQPGGSLRLSCAASGPTFTGSSMSWVR |
| | | QAPGKGLEWVSLISSRGSYTYYADSVKGRFTISRDNSKN |
| | | TLYLQMNSLRAEDTAVYYCARSYAYQYRGLDYWGQG |
| | | TLVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSREEMTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPGK |
| SEQ ID NO: 84 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQNRTSPITFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 85 | CDR1 HC AA | FTFTGSSMSWVR |
| SEQ ID NO: 86 | CDR2 HC AA | VSLISSRGSYTYYADS |
| SEQ ID NO: 87 | CDR3 HC AA | CARSYAYQYRGLDYWGQ |
| SEQ ID NO: 88 | CDR1 LC AA | RASQSVSSSYLA |
| SEQ ID NO: 89 | CDR2 LC AA | GASSRAT |
| SEQ ID NO: 90 | CDR3 LC AA | CQQNRTSPITFGQ |

TABLE 12
Sequences of Anti-CCR6 Antibody MSM R612

| Sequence | | |
|---|---|---|
| Id No: | Sequence of: | Sequence |
| SEQ ID NO: 91 | VH DNA | GAAGTTCAACTGCTGGGGTCCGGTGGTGGTCTGGTAC |
| | | AGCCGGGTGGTTCTCTGCGTCTGAGTTGCGCGGCCAG |
| | | TGGCTTTACCTTCACTACCTATTATATGCATTGGGTGC |
| | | GTCAGGCTCCGGGCAAAGGTCTGGAATGGGTTAGCCT |
| | | TATTTCGCCGAATGCGGGCAATACCTACTATGGGGAT |
| | | AGCGTGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGAGGC |
| | | CGGFCGAGGGCGGGGACTTCCGGCATGGACTACTGGG |
| | | GCCAGGGAACCTTGGTCACCGTCTCG |
| SEQ ID NO: 92 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAAGCTGCTTCTTCTCCTATCACGTTCG |
| | | GCCAAGGGACCAAGGTGGAAATCAAA |
| SEQ ID NO: 93 | HC AA | EVQLLGSGGGLVQPGGSLRLSCAASGFTFTTYYMHWVR |
| | | QAPGKGLEWVSLISPNAGNTYYADSVKGRFTISRDNSK |
| | | NTLYLQMNSLRAEDTAVYYCARGRSRAGTSGMDYWG |
| | | QGTLVTVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIAV |
| | | EWESNGQPENNYKTTPPVLDSDGSFFLYSKLTVDKSRW |
| | | QQGNVFSCSVMHEALHNHYTQKSLSLSPGK |
| SEQ ID NO: 94 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQAASSPITFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 95 | CDR1 HC AA | FTFTTYYMHWVR |
| SEQ ID NO: 96 | CDR2 HC AA | VSLISPNAGNTYYADS |
| SEQ ID NO: 97 | CDR3 HC AA | CARGRSRAGTSGMDYWGQ |
| SEQ ID NO: 98 | CDR1 LC AA | RASQSVSSSYLA |
| SEQ ID NO: 99 | CDR2 LC AA | GASSRAT |
| SEQ ID NO: 100 | CDR3 LC AA | CQQAASSPITFGQ |

TABLE 13
Sequences of Anti-CCR6 Antibody MSM R614

| Sequence | | |
|---|---|---|
| Id No: | Sequence of: | Sequence |
| SEQ ID NO: 101 | VH DNA | GAAGTGCAACTGCTGGAGTCCGGTGGTGGTCTGGTAC |
| | | AGCCGGGTGGTTCTCTGCGTCTGAGTTGCCCGGCCAG |
| | | TGGCTTTACCTTCACTAGATCTGCTATGAGCTGGGTGC |
| | | GTCAGGCTCCGGGCAAAGGTCTGGAATGGGTTAGCTA |
| | | TATTAATTCGTCTGGTGGCGGTACCTACTATGCGGAT |
| | | AGCGTGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGAGCG |
| | | AGGTTCATCTGGGGGTTCGACTACTGGGGCCAGGGAA |
| | | CCCTGGTCACCGTCTCG |
| SEQ ID NO: 102 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTGTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATGGTATTGGGCTCCTGTCACGTTCG |
| | | GCCAAGGGACCAAGGTGGAAATCAAA |
| SEQ ID NO: 103 | HC AA | EVQLLESGGGLVQPGGSLRLSCPASGFTFTRSAMSWVR |
| | | QAPGKGLEWVSYINSSGGGTYYADSVKGRFTISRDNSK |
| | | NTLYLQMNSLRAEDTAVYYCARARFIWGFDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| SEQ ID NO: 104 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQWYWAPVTFGQGTKVEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| SEQ ID NO: 105 | CDR1 HC AA | FTFTRSAMSWVR |
| SEQ ID NO: 106 | CDR2 HC AA | VSYINSSGGGTYYADS |
| SEQ ID NO: 107 | CDR3 HC AA | CARARFTWGFDYWGQ |
| SEQ ID NO: 108 | CDR1 LC AA | RASQSVSSSYLA |
| SEQ ID NO: 109 | CDR2 LC AA | GASSRAT |
| SEQ ID NO: 110 | CDR3 LC AA | CQQWYWAPVTFGQ |

TABLE 14
Sequences of Anti-CCR6 Antibody MSM R615

| Sequence | | |
|---|---|---|
| Id No: | Sequence of: | Sequence |
| SEQ ID NO: 111 | VH DNA | GAAGTTCAACTGCTGGAGTCCGGTGGTGGTCTGGTAC |
| | | AGCCGGGTGGTTCTCTGCGTCTGAGTTGCCTCGGCCAG |
| | | TGGCTTTACCTTCAGTAGCTATTATATGCATTGGGTGC |
| | | GTCAGACTCCGGGCAAAGGTCTGGAATGGGTTAGCAT |
| | | TATTAGTCCGTGGAGTAGCAGTACCTACTATGCGGAT |
| | | AGCGTGAAAGGCCGTTTACCATTTCTCGCGACAACA |
| | | GCAAGAACACGCTGTACCTGCAGATGAACTCACTGCG |
| | | TGCCGAAGATACGGCCGTGTATTACTGTGCGAGATCT |
| | | TCTTACTCTTGGGGTGAATCTTACAAACGTGCCATCG |
| | | ACTACTGGGGCCAGGGAACCTTGGTCACCGTCTCG |
| SEQ ID NO: 112 | VL DNA | GAAATTGTGCTGACCCAGTCTCCGGGCACGTTATCTC |
| | | TGAGCCATGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAC3CCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATCTTATTATTCTCCTGTCACGTTCG |
| | | GCCAAGGGACCAAGGTGGAAATCAAA |
| SEQ ID NO: 113 | HC AA | EVQLLESGGGLVQPGGSLRLSCAASGFTFSSYYMHWVR |
| | | QTPGKGLEWVSIISPWSSSTYYADSVKGRFTISRDNSKNT |
| | | LYLQMNSLRAEDTAVYYCARSSYSWGESYKRAIDYWG |
| | | QGTLVTVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIAV |
| | | EWESNGQPENNYKTTPPVLDSDGSFFLYSKLTVDKSRW |
| | | QQGNVFSCSVMHEALHNHYTQKSLSLSPGK |
| SEQ ID NO: 114 | LC AA | EIVLTQSPGTLSLSHGERATLSCRASQSVSSSYLAWYQQ |
| | | KPGQAPRLLIYGASSRATGIPDRFSGSGSGTDFTLTISRLE |
| | | PEDFAVYYCQQSYYSPVTFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 115 | CDR1 He AA | FTFSSYYMHWVR |
| SEQ ID NO: 116 | CDR2 HC AA | VSIISPWSSSTYYADS |
| SEQ ID NO: 117 | CDR3 HC AA | CARSSYSWGESYKRAIDYWGQ |
| SEQ ID NO: 118 | CDR1 LC AA | RASQSVSSSYLA |
| SEQ ID NO: 119 | CDR2 LC AA | GASSRAT |
| SEQ ID NO: 120 | CDR3 LC AA | CQQSYYSPVTFGQ |

TABLE 15
Sequences of Anti-CCR6 Antibody MSM R617

| Sequence | | |
|---|---|---|
| Id No: | Sequence of: | Sequence |
| SEQ ID NO: 121 | VH DNA | GAAGTTCAACTGCTGGAGTCCGGTGGTGGTCTGGTAC |
| | | AGCCGGGTGGTTCTCTGCGTCTGAGTTGCGCGGCCAG |
| | | TGGCTTTACCTTCAGTGGCTATGCTATGCATTGGGTGC |
| | | GTCAGGCTCCGGGCAAAGGTCTGGAATGGGTTAGCTC |
| | | TATTGGTTCGCGTAGGGGCTTTACCTCTTATGCGGATA |
| | | GCGTGAAAGGCCGTTTTACCATTTCTCGCGACAACAG |
| | | CAAGAACACGCTGTACCTGCAGATGAACTCACTGCGT |
| | | GCCGAAGATACGGCCGTGTATTACTGTGCGAGATCTT |
| | | ACGCTTACCAGTACCGTGGCTTGGACTACTGGGGCCA |
| | | GGGAACCTTGGTCACCGTCTCG |
| SEQ ID NO: 122 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGGGGTGTA |
| | | TTACTGCCAGCAAAGGTCGTCTGTCACGTTCGGCCAA |
| | | GGGACCAAGGTGGAAATCAAA |
| SEQ ID NO: 123 | HC AA | EVQLLESGGGLVQPGGSLRLSCAASGFTFSGYAMHWVR |
| | | QAPGKGLEWVSSIGSRRGFTSYADSVICGRFTISRDNSKN |
| | | TLYLQMNSLRAEDTAVYYCARSYAYQYRGLDYWGQG |
| | | TLVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDVS |
| | | HEDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSREEMTKNQVSLTCLVKGFYPSDIAVEW |
| | | ESNGQPENNYKTTPPVLDSDGSFFLYSKLTVDKSRWQQ |
| | | GNVFSCSVMHEALHNHYTQKSLSLSPGK |
| SEQ ID NO: 124 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQRSSVTFGQGTKVEIKRTVAAPSVFIFPPS |
| | | DEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| SEQ ID NO: 125 | CDR1 HC AA | FTFSGYAMHWVR |
| SEQ ID NO: 126 | CDR2 HC AA | VSSIGSRRGFTSYADS |
| SEQ ID NO: 127 | CDR3 HC AA | CARSYAYQYRGLDYWGQ |
| SEQ ID NO: 128 | CDR1 LC AA | RASQSVSSSYLA |
| SEQ ID NO: 129 | CDR2 LC AA | GASSRAT |
| SEQ ID NO: 130 | CDR3 LC AA | CQQRSSVTFGQ |

TABLE 16
Sequences of Anti-CCR6 Antibody MSM R619

| Sequence | | |
|---|---|---|
| Id No: | Sequence of: | Sequence |
| SEQ ID NO: 131 | VH DNA | GAAGTTCAACTGCTGGAGTCCGGTGGTGGTCTGGTAC |
| | | AGCCGGGTGGTTCTCTGCGTCTGAGTTGCGCGGCCAG |
| | | TGGCTTTACCTTCAGTAACTATTATATCCATTGGGTGC |
| | | GTCAGGCTCCGGGCAAAGGTCTGGAATGGGTTAGCCT |
| | | GATTTCTCCGGGTGGTAGCACTACCTACTATGCGGAT |
| | | AGCGTGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGAGGG |
| | | AGGACTTGGTGGTCGACTTACTATTCCGGCATTGACT |
| | | ACTGGGGCCAGGGAACCTTGGTCACCGTCTCG |
| SEQ ID NO: 132 | VL DNA | GAAATTGTGCTGACCCAGTCTCCGGGGACGTTATCTC |
| | | TGAGCCCTGGTGAGCGCGCCACTCTGTCATGCGGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATATTCTTCTTCTCCTATCACGTTCG |
| | | GCCAAGGGACCAAGGTGGAAATCAAA |
| SEQ ID NO: 133 | HC AA | EVQLLESGGGLVQPGGSLRLSCAASGFTFSNYYIHWVR |
| | | QAPGKGLEWVSLISPGGSTTYYADSVKGRFTISRDNSKN |
| | | TLYLQMNSLRAEDTAVYYCARGRTWWSTYYSGIDYWG |
| | | QGTLVTVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIAV |
| | | EWESNGQPENNYKTTPPVLDSDGSFFLYSKLTVDKSRW |
| | | QQGNVFSCSVMHEALHNHYTQKSLSLSPGK |
| SEQ ID NO: 134 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQYSSSPITFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 135 | CDR1 HC AA | FTFSNYYIHWVR |
| SEQ ID NO: 136 | CDR2 HC AA | VSLISPGGSTTYYADS |
| SEQ ID NO: 137 | CDR3 HC AA | CARGRTWWSTYYSGIDYWGQ |
| SEQ ID NO: 138 | CDR1 LC AA | RASQSVSSSYLA |
| SEQ ID NO: 139 | CDR2 LC AA | GASSRAT |
| SEQ ID NO: 140 | CDR3 LC AA | CQQYSSSPITFGQ |

TABLE 17
Sequences of Anti-CCR6 Antibody MSM R621

| Sequence | | |
|---|---|---|
| Id No: | Sequence of: | Sequence |
| SEQ ID NO: 141 | VH DNA | GAAGTTCAACTGCTGGAGTCCGGTGGTGGTCTGGTAC |
| | | AGCCGGGTGGTTCTCTGCGTCTGAGTTGTGCGGCCAG |
| | | TGGCTTTACCTTCACTACCTATTATATGCATTGGGTGC |
| | | GTCAGGCTCCGGGCAAAGGTCTGGAATGGGTTAGCGG |
| | | TATTGGTCCGCGTGGGAGCTGGACCGGTTATGCGGAT |
| | | AGCGTGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGAGGC |
| | | AGGGCGTTGGTGTCGAATTATTCCGGCTTGGACTACT |
| | | GGGGCCAGGGAACCTTGGTCACCGTCTCG |
| SEQ ID NO: 142 | VL DNA | GAAATTGTGCTGACCCAGTCTCCGGGCACGTTATCTC |
| | | TGAGCCCTGGTGAGCGCGCCACTCTGTCATGCCGGGC |
| | | TTCTVAAAGTGTTACCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATATTATTCTTCTCCTGTCACGTTCG |
| | | GCCAAGGGACCAAGGTGGAAATCAAA |
| SEQ ID NO: 143 | HC AA | EVQLLESGGGLVQPGGSLRLSCAASGFTFTTYYMHWVR |
| | | QAPGKGLEWVSGIGPRGSWTGYADSVKGRFTISRDNSK |
| | | NTLYLQMNSLRAEDTAVYYCARGRALVSNYSGLDYWG |
| | | QGTLVTVSSASTKGPSVFPLAPSSKSTSGGTAALGCLVK |
| | | DYFPEPVTVSWNSGALTSGVHTFPAVLQSSGLYSLSSVV |
| | | TVPSSSLGTQTYICNVNHKPSNTKVDKRVEPKSCDKTHT |
| | | CPPCPAPELLGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIAV |
| | | EWESNGQPENNYKTTPPVLDSDGSFFLYSKLTVDKSRW |
| | | QQGNVFSCSVMHEALHNHYTQKSLSLSPGK |
| SEQ ID NO: 144 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQYYSSPVTFGQGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 145 | CDR1 HC AA | FTFTTYYMHWVR |
| SEQ ID NO: 146 | CDR2 HC AA | VSGIGPRGSWTGYADS |
| SEQ ID NO: 147 | CDR3 HC AA | CARGRALVSNYSGLDYWGQ |
| SEQ ID NO: 148 | CDR1 LC AA | RASQSVSSSYLA |
| SEQ ID NO: 149 | CDR2 LC AA | GASSRAT |
| SEQ ID NO: 150 | CDR3 LC AA | CQQYYSSPVTFGQ |

TABLE 18
Sequences of Anti-CCR6 Antibody MSM R622

| Sequence | | |
|---|---|---|
| Id No: | Sequence of: | Sequence |
| SEQ ID NO: 151 | VH DNA | GAAGTTCAACTGCTGGAGTCCGGTGGTGGTGTGGTACAGC |
| | | CGGGTGGTTCTCTGCGTGTGAGTTGCGCAGCCAGTGGCTTT |
| | | ACCTTCAGTAAAAACTCTATGAGCTGGGTGCGTCAGGCTC |
| | | CGGGCAAAGGTCTGGAATGGGTTAGCCTTATTACTTCGTA |
| | | TTCTGGCTATACCTACTATGCGGATAGCGTGAAAGGCCGT |
| | | TTTACCATTTCTGGCGACAACAGCAAGAACACGCTGTACC |
| | | TGCAGATGAACTCACTGCGTGCCGAAGATACGGCCGTGTA |
| | | TTACTGTGCGAGATCTTACGCTTACCAGTACCGTGGCTTGG |
| | | ACTACTGGGGCCAGGGAACCTTGGTCACCGTCTCG |
| SEQ ID NO: 152 | VL DNA | GAAATTGTGGTGACCCAGTCTCCGGGCACGTTATCTCTGA |
| | | GCCCTGGTGAGCGCGCCACTCTGTCATGCCGGGCTTCTCA |
| | | AAGTGTTAGCAGTAGCTACCTGGCGTGGTATCAGCAAAAA |
| | | CCGGGCCAGGCCCCGCGTCTGCTGATTTACGGTGCATCCA |
| | | GCCGTGCCACCGGCATTCCAGATCGTTTTTCCGGTAGTGGT |
| | | TCTGGGACGGACTTCACTCTGACAATCTCACGCCTGGAAC |
| | | CGGAGGATTTTGCGGTGTATTACTGCCAGCAAAATGGGAG |
| | | GAGTCCTGTCACGTTCGGCCAAGGGACCAAGGTGGAAATC |
| | | AAA |
| SEQ ID NO: 153 | HC AA | EVQLLESGGGLVQPGGSLRLSCAASGFTFSKNSMSWVRQAP |
| | | GKGLEWVSLITSYSGYTYYADSVKGRFTISRDNSKNTLYLQ |
| | | MNSLRAEDTAVYYCARSYAYQYRGLDYWGQGTLVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPELLGGPSVFLFPPKP |
| | | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPAPIEKTISKAKGQPREPQVYTLPPSREEMTKNQVSLTCLVK |
| | | GFYPSDIAVEWESNGQPENNYKTTPPVLDSDGSFFLYSKLTV |
| | | DKSRWQQGNVFSCSVMHEALHNHYTQKSLSLSPGKDKSRW |
| | | QQGNVFSCSVMHEALHNHYTQKSLSLSPGK |
| SEQ ID NO: 154 | LC AA | EIVLTQSPGTLSLSPGERATLSCRASQSVSSSYLAWYQQKPGQ |
| | | APRLLIYGASSRATGIPDRFSGSGSGTDFTLTISRLEPEDFAVY |
| | | YCQQNGRSPVTFGQGTKVEIKRTVAAPSVFIFPPSDEQLKSGT |
| | | ASVVCLLNNFYPREAKVQWKVDNALQSGNSQESVTEQDSK |
| | | DSTYSLSSTLTLSKADYEKHKVYACEVTHQGLSSPVTKSFNR |
| | | GEC |
| SEQ ID NO: 155 | CDR1 HC AA | FTFSKNSMSWVR |
| SEQ ID NO: 156 | CDR2 HC AA | VSLITSYSGYTYYADS |
| SEQ ID NO: 157 | CDR3 HC AA | CARSYAYQYRGLDYWGQ |
| SEQ ID NO: 158 | CDR1 LC AA | RASQSVSSSYLA |
| SEQ ID NO: 159 | CDR2 LC AA | GASSRAT |
| SEQ ID NO: 160 | CDR3 LC AA | CQQNGRSPVTFGQ |

TABLE 19
Sequences of Anti-CCR6 Antibody MSM R623

| Sequence | | |
|---|---|---|
| Id No: | Sequence of: | Sequence |
| SEQ ID NO: 161 | VH DNA | GAAGTTCAACTGCTGGAGTCCGGrGGTGGTCTGGTAC |
| | | AGCCGGGTGGTTCTCTGCGTCTGAGTTGTGCGGCCAG |
| | | TGGCTTTACCTTCAGTAACTATTATATCCATTGGGTGC |
| | | GTCAGGCTCCGGGCAAAGGTCTGGAATGGGTTAGCAC |
| | | GATTGGTTCGAGTAATGGCACGACCTACTATGCGGAT |
| | | AGCGTGAAAGGCCGTTTTACCATTTCTCGCGACAACA |
| | | GCAAGAACACGCTGTACCTGCAGATGAACTCACTGCG |
| | | TGCCGAAGATACGGCCGTGTATTACTGTGCGAGAGGC |
| | | AGGGCGGTGAAGCTGGCTTACTATTCCGGGTTTGACT |
| | | ACTGGGGCCAGGGAACCTTGGTCACCGTCTCG |
| SEQ ID NO: 162 | VL DNA | GAAATTGTGCTGACCCAGTCTCCGGGCACGTTATCTC |
| | | TGAGCCCTGGTGAGCGCGCCACCCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATATTCTTATTCTCCTATCACGTTCG |
| | | GCCAAGGGACCAAGGTGGAAATCAAA |
| SEQ ID NO: 163 | HC AA | EVQLLESGGGLVQPGGSLRLSCAASGFTFSNYYIHWVR |
| | | QAPGKGLEWVSTIGSSNGTTYYADSVKGRFTISRDNSKN |
| | | TLYLQMNSLRAEDTAVYYCARGRAVKLAYYSGFDYW |
| | | GQGTLVTVSSASTKGPSVFPLAPSSKSTSGGTAALGCLV |
| | | KDYFPEPVTVSWNSGALTSGVHTFPAVLQSSGLYSLSSV |
| | | VTVPSSSLGTQTYICNVNHKPSNTKVDKRVEPKSCDKTH |
| | | TCPPCPAPELLGGPSVFLFPPKPKDTLMISRTPEVTCVVV |
| | | DVSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 164 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQYSYSPITFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 165 | CDR1 HC AA | FTFSNYYIHWVR |
| SEQ ID NO: 166 | CDR2 HC AA | VSTIGSSNGTTYYADS |
| SEQ ID NO: 167 | CDR3 HC AA | CARGRAVKLAYYSGFDYWGQ |
| SEQ ID NO: 168 | CDR1 LC AA | RASQSVSSSYLA |
| SEQ ID NO: 169 | CDR2 LC AA | GASSRAT |
| SEQ ID NO: 170 | CDR3 LC AA | CQQYSYSPITFGQ |

TABLE 20
Sequences of Anti-CCR6 Antibody MSM R624

| Sequence | | |
|---|---|---|
| Id No: | Sequence of | Sequence |
| SEQ ID NO: 171 | VH DNA | GAAGTTCAACTGCTGGAGTCCGGTGGTGGTCTGGTACAGC |
| | | CGGGTGGTTCTCTGCGTCTGAGTTGCGCGGCCAGTGGCTTT |
| | | ACCTTCAGTAGCTATGCCATGAGCTGGGTGCGTCAGGCGC |
| | | CGGGCAAAGGTCTGGAATGGGTTAGCGCGATTAGCGGTA |
| | | GTGGCGGTAGCACGTACTATGCGGATAGCGTGAAAGGCC |
| | | GTTTTACCATTTCTCGCGACAACAGCAAGAACACGCTGTA |
| | | CCTGCAGATGAACTCACTGCGTGCCGAAGATACGGCCGTG |
| | | TATTACTGTGCGAGACAGTCTTGGTGGCGTTCTAAAATGA |
| | | TGTACGACTCTCATCTGTCTTACTCTTCTGCTTCTGGCTTCG |
| | | ACTACTGGGGCCAGGGAACCTTGGTCACCGTCTCG |
| SEQ ID NO: 172 | VL DNA | GAAATTGTGTTGACGCAGTCTCCGGGCACGTTATCTCTGA |
| | | GCCCTGGTGAGCGCGCCACTCTGTCATGCCGGGCTTCTCA |
| | | AAGTGTTAGCAGTAGCTACCTGGCGTGGTATCAGCAAAAA |
| | | CCGGGCCAGGCCCCGCGTCTGCTGATTTACGGTGCATCCA |
| | | GCCGTGCCACCGGCATTCCAGATCGTTTTTCCGGTAGT0GT |
| | | TCTGGGACGGACTTCACTCTGACAATCTCACGCCTGGAAC |
| | | CGGAGGATTTTGCGGTGTATTACTGCCAGCAATCTTCTTCT |
| | | TCTCCTATCACGTTCGGCCAAGGGACCAAGGTGGAAATCA |
| | | AA |
| SEQ ID NO: 173 | HC AA | EVQLLESGGGLVQPGGSLRLSCAASGFTFSSYAMSWVRQAP |
| | | GKGLEWVSAISGSGGSTYYADSVKGRFTISRDNSKNTLYLQ |
| | | MNSLRAEDTAVYYCARQSWWRSKMMYDSHLSYSSASGFD |
| | | YWGQGTLVTVSSASTKGPSVFPLAPSSKSTSGGTAALGCLVK |
| | | DYFPEPVTVSWNSGALTSGVHTFPAVLQSSGLYSLSSVVTVP |
| | | SSSLGTQTYICNVNHKPSNTKVDKRVEPKSCDKTHTCPPCPA |
| | | PELLGGPSVFLFPPKPKDTLMISRTPEVTCVVVDVSHEDPEVK |
| | | FNWYVDGVEVHNAKTKPREEQYNSTYRVVSVLTVLHQDWL |
| | | NGKEYKCKVSNKALPAPIEKTISKAKGQPREPQVYTLPPSREE |
| | | MTKNQVSLTCLVKGFYPSDIAVEWESNGQPENNYKTTPPVL |
| | | DSDGSFFLYSKLTVDKSRWQQGNVFSCSVMHEALHNHYTQ |
| | | KSLSLSPGK |
| SEQ ID NO: 174 | LC AA | EIVLTQSPGTLSLSPGERATLSCRASQSVSSSYLAWYQQKPGQ |
| | | APRLLIYGASSRATGIPDRFSGSGSGTDFTLTISRLEPEDFAVY |
| | | YCQQSSSSPITFGQGTKVEIKRTVAAPSVFIFPPSDEQLKSGTA |
| | | SVVCLLNNFYPREAKVQWKVDNALQSGNSQESVTEQDSKDS |
| | | TYSLSSTLTLSKADYEKHKVYACEVTHQGLSSPVTKSFNRGE |
| | | C |
| SEQ ID NO: 175 | CDR1 HC AA | FTFSSYAMSWVR |
| SEQ ID NO: 176 | CDR2 HC AA | VSAISGSGGSTYYADS |
| SEQ ID NO: 177 | CDR3 HC AA | CARQSWWRSKMMYDSHLSYSSASGFDYWGQ |
| SEQ ID NO: 178 | CDR1 LC AA | RASQSVSSSYLA |
| SEQ ID NO: 179 | CDR2 LC AA | GASSRAT |
| SEQ ID NO: 180 | CDR3 LC AA | CQQSSSSPITFGQ |

TABLE 21
Sequences of Anti-CCR6 Antibody MSM R625

| Sequence | | |
|---|---|---|
| Id No: | Sequence of: | Sequence |
| SEQ ID NO: 181 | VH DNA | GAAGTTCAACTGCTGGAGTCCGGTGGTGGTCTGGTAC |
| | | AGCCGGGTGGTTCTCTGCGTCTGAGTTGCGCGGCCAG |
| | | TGGCTTTACCTTCAGTAGCTATGCCATGAGCTGGGTG |
| | | CGTCAGGCGCCGGGCAAAGGTCTGGAATGGGTTAGC |
| | | GCGATTAGCGGTAGTGGCGGTAGCACGTACTATGCGG |
| | | ATAGCGTGAAAGGCCGTTTTACCATTTCTCGCGACAA |
| | | CAGCAAGAACACGCTGTACCTGCAGATGAACTCACTG |
| | | CGTGCCGAAGATACGGCCGTGTATTACTGTGCGAGAC |
| | | AGAACGTTTGGTACGGTGGCTTGGACTACTGGGGCCA |
| | | GGGAACCTTGGTCACCGTCTCG |
| SEQ ID NO: 182 | VL DNA | GAAATTGTGTTGACGCAGTCTCCGGGCACGTTATCTC |
| | | TGAGCCCTGGTGAGCGCGCCACTCTGTCATGCCGGGC |
| | | TTCTCAAAGTGTTAGCAGTAGCTACCTGGCGTGGTAT |
| | | CAGCAAAAACCGGGCCAGGCCCCGCGTCTGCTGATTT |
| | | ACGGTGCATCCAGCCGTGCCACCGGCATTCCAGATCG |
| | | TTTTTCCGGTAGTGGTTCTGGGACGGACTTCACTCTGA |
| | | CAATCTCACGCCTGGAACCGGAGGATTTTGCGGTGTA |
| | | TTACTGCCAGCAATATTCTTATGCTCCTATCACGTTCG |
| | | GCCAAGGGACCAAGGTGGAAATCAAA |
| SEQ ID NO: 183 | HC AA | EVQLLESGGGLVQPGGSLRLSCAASGFTFSSYAMSWVR |
| | | QAPGKGLEWVSAISGSGGSTYYADSVKGRFTISRDNSKN |
| | | TLYLQMNSLRAEDTAVYYCARQNVWYGGLDYWGQGT |
| | | LVTVSSASTKGPSVFPLAPSSKSTSGGTAALGCLVKDYF |
| | | PEPVTVSWNSGALTSGVHTFPAVLQSSGLYSLSSVVTVP |
| | | SSSLGTQTYICNVNHKPSNTKVDKRVEPKSCDKTHTCPP |
| | | CPAPELLGGPSVFLFPPKPKDTLMISRTPEVTCVVVDVSH |
| | | EDPEVKFNWYVDGVEVHNAKTKPREEQYNSTYRVVSV |
| | | LTVLHQDWLNGKEYKCKVSNKALPAPIEKTISKAKGQP |
| | | REPQVYTLPPSREEMTKNQVSLTCLVKGFYPSDIAVEWE |
| | | SNGQPENNYKTTPPVLDSDGSFFLYSKLTVDKSRWQQG |
| | | NVFSCSVMHEALHNHYTQKSLSLSPGK |
| SEQ ID NO: 184 | LC AA | EIVLTQSPGTLSLSPGERATLSCRASQSVSSSYLAWYQQ |
| | | KPGQAPRLLIYGASSRATGIPDRFSGSGSGTDFTLTISRLE |
| | | PEDFAVYYCQQYSYAPITFGQGTKVEIKRTVAAPSVFIFP |
| | | PSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| SEQ ID NO: 185 | CDR1 HC AA | FTFSSYAMSWVR |
| SEQ ID NO: 186 | CDR2 HC AA | VSAISGSGGSTYYADS |
| SEQ ID NO: 187 | CDR3 HC AA | CARQNVWYGGLDYW |
| SEQ ID NO: 188 | CDR1 LC AA | RASQSVSSSYLA |
| SEQ ID NO: 189 | CDR2 LC AA | GASSRAT |
| SEQ ID NO: 190 | CDR3 LC AA | CQQYSYAPITFGQ |

TABLE 22
Sequences of Anti-CCR6 Antibody MSM R626

| Sequence | | |
|---|---|---|
| Id No: | Sequence of: | Sequence |
| SEQ ID NO. 191 | HC AA | EVQLLESGGGLVQPGGSLRLSCAASGFTFIYYYMSWVRQAPGKG |
| | | LEWVSSISPYYGYTSYADSVKGRFTISRDNSKNTLYLQMNSLRAE |
| | | DTAVYYCARRTWWLRGMDYWGQGTLVTVSS |
| SEQ ID NO. 192 | LC AA | EIVLTQSPGTLSLSPGERATLSCRASQSVSSSYLAWYQQKPGQAPR |
| | | LLIYGASSRATGIPDRYSGSGSGTDFTLTISRLEPEDFAVYYCQQSS |
| | | YYPITFGQGTKVEIK |
| SEQ ID NO. 193 | CDR1 HC AA | FTFIYYYMSWVR |
| SEQ ID NO. 194 | CDR2 HC AA | VSSISPYYGYTSYADS |
| SEQ ID NO. 195 | CDR3 HC AA | CARRTWWLRGMDYWGQ |
| SEQ ID NO. 196 | CDR1 LC AA | RASQSVSSSYLA |
| SEQ ID NO. 197 | CDR2 LC AA | GASSRAT |
| SEQ ID NO. 198 | CDR3 LC AA | CQQSSYYPITFGQ |

TABLE 23
Sequences of Anti-CCR6 Antibody MSM R627

| Sequence | | |
|---|---|---|
| Id No: | Sequence of | Sequence |
| SEQ ID NO. 199 | HC AA | EVQLLESGGGLVQPGGSLRLSCAASGFTISSYMAWVRQAPGKGLE |
| | | WVSSISPSSGSTYYADSVKGRFTISRDNSKNTLYLQMNSLRAEDTA |
| | | VYYCARSMWSKQMHRYGLDYWGQGTLVTVSS |
| SEQ ID NO. 200 | LC AA | EIVLTQSPGTLSLSPGERATLSCRASQSVSSSYLAWYQQKPGQAPR |
| | | LLIYGASSRATGIPDRFSGSGSGTDFTLTISRLEPEDFAVYYCQQAS |
| | | SDPITFGQGTKVEIK |
| SEQ ID NO. 201 | CDR1 HC AA | FTISSYMAWVR |
| SEQ ID NO. 202 | CDR2 HC AA | VSSISPSSGSTYYADS |
| SEQ ID NO. 203 | CDR3 HC AA | CARSMWSKQMHRYGLDYWGQ |
| SEQ ID NO. 204 | CDR1 LC AA | RASQSVSSSYLA |
| SEQ ID NO. 205 | CDR2 LC AA | GASSRAT |
| SEQ ID NO. 206 | CDR3 LC AA | CQQASSDPITFGQ |

TABLE 24
Sequences of Anti-CCR6 Antibody MSM R628

| Sequence | | |
|---|---|---|
| Id No: | Sequence of: | Sequence |
| SEQ ID NO. 207 | HC AA | EVQLLESGGGLVQPGGSLRLNCAASGFTFSSYAMSWVRQAPGKG |
| | | LEWVSAISGSGGSTYYADSVKGRFTISRDNSKNTLYLQMNSLRAE |
| | | DTAVYYCAKKGYYGGMAMDYWGQGTLVTVSS |
| SEQ ID NO. 208 | LC AA | EIVLTQSPGTLSLSPGERATLSCRASQSVSSSYLAWYQQKPGQAPR |
| | | LLIYGASSRATGIPDRFSGSGSGTDFTLTISRLEPEDFAVYYCQQYS |
| | | SSPLTFGQGTKVEIK |
| SEQ ID NO. 209 | CDR1 HC AA | FTFSSYAMSWVR |
| SEQ ID NO. 210 | CDR2 HC AA | VSAISGSGGSTYYADS |
| SEQ ID NO. 211 | CDR3 HC AA | CAKKGYYGGMAMDYWGQ |
| SEQ ID NO. 212 | CDR1 LC AA | RASQSVSSSYLA |
| SEQ ID NO. 213 | CDR2 LC AA | GASSRAT |
| SEQ ID NO. 214 | CDR3 LC AA | CQQYSSSPLTFGQ |

TABLE 25
Sequences of Anti-CCR6 Antibody MSM R629

| Sequence | | |
|---|---|---|
| Id No: | Sequence of: | Sequence |
| SEQ ID NO. 215 | HC AA | EVQLLESGGGLVQPGGSLRLSCAASGFTFSSYAMSWVRQAPGKG |
| | | LEWVSAISGSGGSTYYADSVKGRFTISRDNSKNTLYLQMNSLRAE |
| | | DTAVYYCARYHHGMKFGMDYWOQGTLVTVSS |
| SEQ ID NO. 216 | LC AA | EIVLTQSPGTLSLSPGERATLSCRASQSVSSSYLAWYQQKPGQAPR |
| | | LLIYGASSRATGIPDRFSGSGSGTDFTLTISRLEPEDFAVYYCQQSS |
| | | SSPITFGQGTKVEIK |
| SEQ ID NO. 217 | CDR1 HC AA | FTFSSYAMSWVR |
| SEQ ID NO. 218 | CDR2 HC AA | VSAISGSGGSTYYADS |
| SEQ ID NO. 219 | CDR3 HC AA | CARYHHGMKFGMDYWGQ |
| SEQ ID NO. 220 | CDR1 LC AA | RASQSVSSSYLA |
| SEQ ID NO. 221 | CDR2 LC AA | GASSRAT |
| SEQ ID NO. 222 | CDR3 LC AA | CQQSSSSPITFGQ |

TABLE 26
Sequences of Anti-CCR6 Antibody MSM R630

| Sequence | | |
|---|---|---|
| Id No: | Sequence of: | Sequence |
| SEQ ID NO. 223 | HC AA | EVQLLESGGGLVQPGGSLRLNCAASGFTFSSYAMSWVRQAPGKG |
| | | LEWVSAISGSGGSTYYADSVKGRFTISRDNSKNTLYLQMNSLRAE |
| | | DTAVYYCARKSYYSWPALDYWGQGTLVTVSS |
| SEQ ID NO. 224 | LC AA | |
| SEQ ID NO. 225 | CDR1 HC AA | FTFSSYAMSWVR |
| SEQ ID NO. 226 | CDR2 HC AA | FTFSSYAMSWVR |
| SEQ ID NO. 227 | CDR3 HC AA | CARKSYYSWPALDYWGQ |
| SEQ ID NO. 228 | CDR1 LC AA | RASQSVSSSYLA |
| SEQ ID NO. 229 | CDR2 LC AA | GASSRAT |
| SEQ ID NO. 230 | CDR3 LC AA | |

TABLE 27
Sequences of Anti-CCR6 Antibody MSM R631

| Sequence | | |
|---|---|---|
| Id No: | Sequence of: | Sequence |
| SEQ ID NO. 231 | HC AA | EVQLLESGGGLVQPGGSLRLSCAASGFTITAYAMAWVRQAPGKG |
| | | LEWVSYISSDSGYTYYADIVKGRFTISRDNSKNTLYLQMNSLRAE |
| | | DTAVYYCARSLYSRRSSSRGMDYWGQGTLVTVSS |
| SEQ ID NO. 232 | LC AA | EIVLTQSPGTLSLSPGERATLSCRASQSVSSSYLAWYQQKPGQAPR |
| | | LLIYGASSRATGIPDRFSGSGSGTDFTLTISRLEPEDFAVYYCQQSY |
| | | SAPVTFGQGTKVEIK |
| SEQ ID NO. 233 | CDR1 HC AA | FTITAYAMAWVR |
| SEQ ID NO. 234 | CDR2 HC AA | VSYISSDSGYTYYADI |
| SEQ ID NO. 235 | CDR3 HC AA | CARSLYSRRSSSRGMDYWGQ |
| SEQ ID NO. 236 | CDR1 LC AA | RASQSVSSSYLA |
| SEQ ID NO. 237 | CDR2 LC AA | GASSRAT |
| SEQ ID NO. 238 | CDR3 LC AA | CQQSYSAPVTPGQ |

TABLE 28
Sequences of Anti-CCR6 Antibody MSM R632

| Sequence | | |
|---|---|---|
| Id No: | Sequence of: | Sequence |
| SEQ ID NO. 239 | HC AA | EVQLLESGGGLVQPGGSLRLSCAASGFTLTAYSISWVRQAPGKGL |
| | | EWVSSIYSYSGSTYYADSVKGRFTISRDNSKNTLYLQMNSLRAED |
| | | TAVYYCARYYSYSYAMDYWGQGTLVTVSS |
| SEQ ID NO. 240 | LC AA | EIVLTQSPGTLSLSPGERATLSCRASQSVSSSYLAWYQQKPGQAPR |
| | | LLIYGASSRATDIPDRFSGSGSGTDFTLTISRLEPEDFAVYYCQQYS |
| | | YSPVTFGQGTKVEIK |
| SEQ ID NO. 241 | CDR1 HC AA | FTLTAYSISWVR |
| SEQ ID NO. 242 | CDR2 HC AA | VSSIYSYSGSTYYADS |
| SEQ ID NO. 243 | CDR3 HC AA | CARYYSYSYAMDYWGQ |
| SEQ ID NO. 244 | CDR1 LC AA | RASQSVSSSYLA |
| SEQ ID NO. 245 | CDR2 LC AA | GASSRAT |
| SEQ ID NO. 246 | CDR3 LC AA | CQQYSYSPVTFGQ |

TABLE 29
Sequences of Anti-CCR6 Antibody MSM R633

| Sequence | | |
|---|---|---|
| Id No: | Sequence of: | Sequence |
| SEQ ID NO. 247 | HC AA | EVQLLESGGGLVQPGGSLRLSCAASGFTFTSSYMAWVRQAPGKG |
| | | LEWVSSIAPSSGSTYYADSVKGRFTISRDNSKNTLYLQMNSLRAE |
| | | DTAVYYCARSRWEWYYHSSHGLDYWGQGTLVTVSS |
| SEQ ID NO. 248 | LC AA | EIVLTQSPGTLSLSPGERATLSCRASQSVSSSYLAWYQQKPGQAPR |
| | | LLIYGASSRATDIPDRFSGSGSGTDFTLTISRLEPEDFAVYYCQQSY |
| | | SDPITFGQGTKVEIK |
| SEQ ID NO. 249 | CDR1 HC AA | FFFTSSYMAWVR |
| SEQ ID NO. 250 | CDR2 HC AA | VSSIAPSSGSTYYADS |
| SEQ ID NO. 251 | CDR3 HC AA | CARSRWEWYYHSSHGLDYWGQ |
| SEQ ID NO. 252 | CDR1 LC AA | RASQSVSSSYLA |
| SEQ ID NO. 253 | CDR2 LC AA | GASSRAT |
| SEQ ID NO. 254 | CDR3 LC AA | CQQSYSDPITFGQ |

TABLE 30
Sequences of Anti-CCR6 Antibody MSM R634

| Sequence | | |
|---|---|---|
| Id No:. | Sequence of: | Sequence |
| SEQ ID NO. 255 | HC AA | EVQLLESGGGLVQPGGSLRLSCAASGFTFTTYYMSWVRQAPGKG |
| | | PEWVSNIAAGGATDYADSVKGRFTISRDNSKNTLYLQMNSLRAE |
| | | DTAVYYCARGPWGRYHPMGFDYWGQGTLVTVSS |
| SEQ ID NO. 256 | LC AA | EIVLTQSPGTLSLSPGERATLSCRASQSVSSSYLAWYQQKPGQAPR |
| | | LLIYGASSRATDIPDRFSGSGSGTDFTLTISRLEPEDFAVYYCQQAY |
| | | YSPVTFGQGTKVEIK |
| SEQ ID NO. 257 | CDR1 HC AA | FIFTTYYMSWVR |
| SEQ ID NO. 258 | CDR2 HC AA | VSNIAAGGATDYADS |
| SEQ ID NO. 259 | CDR3 HC AA | CARGPWGRYHPMGFDYWGQ |
| SEQ ID NO. 260 | CDR1 LC AA | RASQSVSSSYLA |
| SEQ ID NO. 261 | CDR2 LC AA | GASSRAT |
| SEQ ID NO. 262 | CDR3 LC AA | CQQAYYSPVTFGQ |

### Example 16

### Derivatives of Anti-CCR6 Antibodies

Another embodiment of this invention relates to anti-CCR6 antibodies obtained by changing one or several amino acids in CDRs by means of site directed mutagenesis. Those skillful in the art are well familiar with and often implement such an approach for generating a pool of derivative antibodies in anticipation that among so generated antibodies can be antibodies that are more suitable from the point of view of their manufacturability, storage and general stability, binding characteristics, immunogenicity, etc. Such derivatives of this invention retain or outperform certain binding characteristics. Other amino acids can be changed this way. So obtained antibodies for the CCR6 target can be generated, purified, and characterized to obtain derivative antibodies with improved properties. In general, derivative antibodies for the CCR6 target can have more than 80% homology as defined using either BLOSUM62 or PAM250 similarity matrix in HCDR3 alone or LCDR3+HCDR1+HCDR2 cumulatively as compared with an existing anti-CCR6 antibody of this invention. These derivative antibodies are also an embodiment of the invention.

In addition to the specifically identified antibodies shown in Tables 2-9, variations of these sequences can also be used. Conservative substitution of certain amino acids does not adversely affect binding of antibodies to their targets. Such conservative substitutions are shown below in Table 31.

TABLE 31
Conservative Amino Acid Substitutions

| Feature | Substituting Amino Acids |
|---|---|
| Basic side chains: | arginine, histidine, lysine |
| Acidic side chains: | aspartic acid, glutamic acid |
| Uncharged polar | asparagine, cysteine, glutamine, glycine, |
| side chains: | serine, threonine, tyrosine, tryptophan |
| Nonpolar side chains: | alanine, isoleucine, leucine, phenylalanine, |
| | proline, methionine, valine |
| Branched side chains: | isoleucine, threonine, valine |
| Aromatic side chains: | histidine, tyrosine, phenalalanine, tryptophan |

Another approach to generating antibodies for the same target of heightened properties from an original antibody is changing amino acids similar to that known for naturally occurring somatic mutations in other than CDRs sequence regions. A possible drawback of such changes can be enhanced immunogenicity of the antibodies, which can be analyzed by a combination of analytical tools and experimentally (such services are commercially available) and potentially immunogenic antibodies discarded. This approach is known to those skillful in the art as germlining and derivatives so produced also represent an embodiment of this invention.

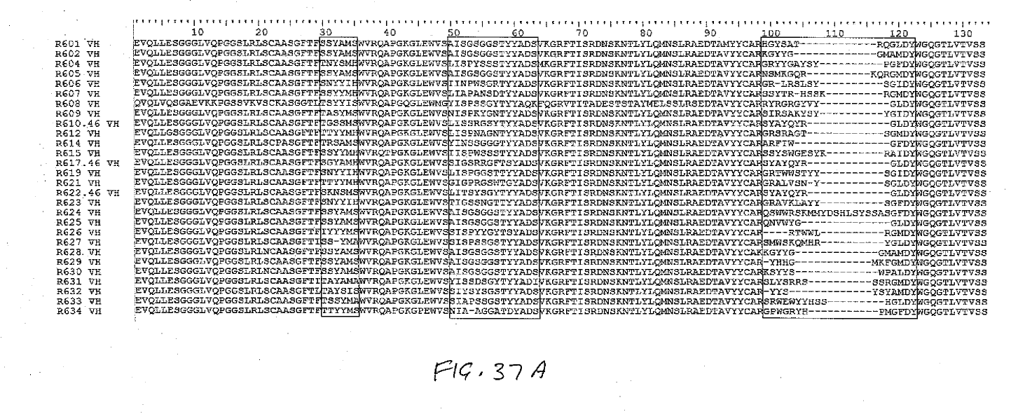

FIGS. 37A and 37B depict sequence alignments for CCR6 variable heavy chains (VH) and variable light chains (VL) of this invention. As shown in the outlined boxes of FIGS. 37A and 37B, for CDR1 (loft boxes), CDR2 (middle boxes) and CDR3 (right boxes), VL sequences are nearly all identical, with some variations in CDR3 VL. The CDR3 regions of VH show significant differences, which we believe to be largely responsible for the binding and activity of the antibodies of this invention. However, there are also some variations in sequences of CDRs 1 and 3 that may contribute to binding and activity.

### Example 17

### Binding of CCR6 Antibodies to PBMCs or Splenocytes

To determine whether CCR6 antibodies of this invention bind to peripheral blood mononuclear cells (PBMCs) or splenocytes, we carried out a series of studies in which PBMCs or splenocytes (10,000 per well) were incubated with 100M IgG-bio in FACS buffer (20 μl total volume per well) in 40 min at 4° C. Than cells were washed twice and stained in 20 min with

a. A mixture of antihuman CD4-PerCP conjugate (BioLegend, #317432) and Streptavidin-PE conjugate (R&D systems, #F0040) both diluted 1:50 in FACS (10 μl per well)—samples of Human PBMC and Cyno PBMC;

b. or a mixture of anti-mouse CD4-PE-Cy5 conjugate (eBioscience, #15-0041) and Streptavidin-PE conjugate (R&D systems, #P0040) both diluted 1:50 in FACS (10 μl per well)—samples of Mouse splenocytes.

c. Finally, cells were washed twice and fixed in FIX (75 μl per well)

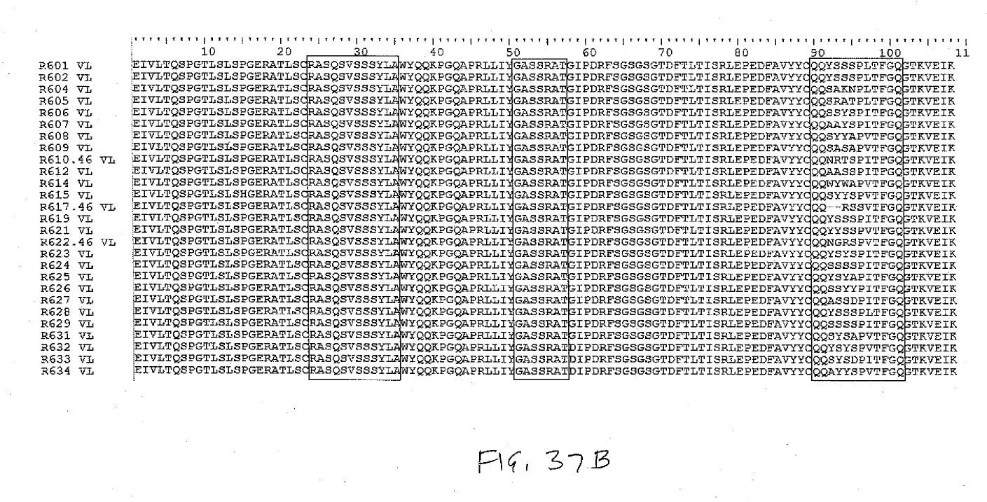

The MFI of the cells was measured by GUAVA PCA-96 at 485V on 580 nm channel and 490V on 675 nm channel (measurements were made in 2 repeats). Results of these studies is shown in FIGS. 38A-38C. FIG. 38A depicts binding of R605 IgG1 antibodies against CCR6 to human PBMCs. FIG. 38B depicts binding of MSM R605 antibodies to PMBCx of Cynomologus monkeys, and FIG. 38C depicts binding of MSM R605 antibodies to mouse splenocytes.

In another study, the amount of IgG stained cells in CD4-positive pool was calculated as function.

S1=(x1/T1)×100%;

where x1 is number of CCR7-positive/CD4-positive cells; T1 is total number of CD4-positive cells. The amount of IgG stained cells in CD4-negative pool was calculated as function

S2=(x2/T2)×100%;

where x2 is number of CCR7-positive/CD4-negative cells; 72 is total number of CD4-negative cells.

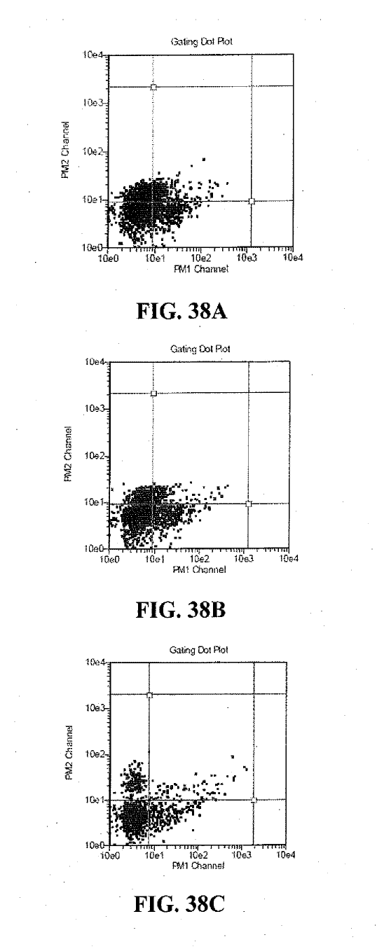

Results of this study are shown in FIG. 39. FIG. 39 depicts histograms of the number of IgG stained cells (vertical axis) vs. the cell fractions studied. For each species, both CD4-positive and CD4-negative cells were labeled by MSM R605 of this invention. However, in each case, more CD4-positive cells stained than CD4-negative cells. For human PBMCs (left two columns), the difference between CD4-positive and CD4-negative cells were quite small, and the difference was neither substantial nor statistically significant. For the Cynomologus monkey PBMCs (middle two columns), the difference was somewhat greater than for the human PBMCs, and as with the human PBMCs, the difference between CD4-positive and CD4-negative cell was neither substantial nor statistically significant. In contrast, for mouse splenocytes (right two columns), more CD4-positive cells stained with MSM R605 than CD4-negative cells. The difference was substantial, and was statistically significant.

We conclude from these studies that MSM R605 selectively labels CD4-positive and CD4-negative cells in each species, and therefore, studies in monkeys and mice are reasonably predictive of results obtained in human beings.

### Example 19

### Properties of Anti-CCR6 Antibodies

To determine effectiveness of anti-CCR6 antibodies of this invention, we carried out a series of binding studies.

Table 32 below summarizes binding data (EC₅₀) and inhibition of calcium flux (IC₅₀) for antibodies of this invention MSM R601 through MSM R623.

TABLE 32
Properties of MSM Anti-CCR6 Antibodies

| | Binding EC₅₀, nM | | | | |
|---|---|---|---|---|---|
| | CCR6 | CCR6 | CCR6 | Ca-Flux IC₅₀, nM | |
| Mep-R6 | human | cyno | mouse | CCR6/CCL20 | Specificity |
| MSM R601 | 25 | 14.5 | n/b | n/f | OK |
| MSM R602 | 11 | 5 | n/b | n/f | OK |
| MSM R604 | 2.9 | 1.5 | n/b | n/f | OK |
| MSM R605 | 1.0 | 1.8 | 1.6 | 1.5 | Cross |
| | | | | | CXCR6, CCR7ms, CCR9 |
| MSM R606 | 1.4 | 1.7 | 0.7 | 8.3 | Slightly sticky |
| MSM R607 | 1.2 | 2.0 | 1.0 | 13.5 | Cross CXCR6, sticky |
| MSM R608 | 2.0 | 2.5 | 1.0 | n/f | OK |
| MSM R609 | 1.0 | 1.6 | >100 | n/f | OK |
| MSM R610.46** | >100 | >100 | n/b | n/f | |
| MSM R612 | 1.0 | 1.6 | 1.5 | n/f | OK |
| MSM R614^{†} | 2.5 | 3.0 | n/b | n/f | Cross CXCR2 |
| MSM R615 | 1.0 | 2.5 | n/b | n/f | OK |
| MSMR617.46 | 1.5 | 1.9 | >100 | n/f | OK |
| MSM R19 | 1.7 | 2.0 | n/b | n/f | OK |
| MSm R621^{†} | 1.5 | 2.2 | 30 | n/f | Cross CXCR6? |
| MSM R622.46 | 1.9 | 1.7 | >100 | n/f | Cross CXCR6? |
| MSM R623 | 2.8 | 3.2 | 1.8 | n/f | OK |

Table 33 below shows binding data and calcium flux data for anti-CCR5 antibodies MSM 624 through MSM R633

TABLE 33
Binding and Calcium Flux Inhibition of Anti-CCR6 Antibodies

| | EC50, nM | | | IC₅₀, nM | |
|---|---|---|---|---|---|
| | CCR6 | CCR6 | CCR6 | (Ca-Flux) | |
| MSM No. | human | cyno | mouse | CCR6/CCL20 | Specificity |
| MSM-R624 | 1.8 | 3 | n/b | n/f | OK |
| MSM-R625 | 1.6 | 2.6 | n/b | n/f | Cross X2 |
| MSM-R627 | | | | | |
| MSM-R628 | 1.8 | 1.3 | n/b | n/f | OK |
| MSM-R629 | | | | | |
| MSM-R630 | | | | | |
| MSM-R631 | | | | | |
| MSM-R632 | | | | | |
| MSM-R633 | 7 | 7.5 | n/b | n/f | OK |
| MSM-R634 | | | | | |
| MSM-R627/33 | 2.6 | 2.9 | >500 | >500 | OK |

### Example 19

### Fully Human Antibodies Against Human CCR6 in IgG4 Format

Antibodies of this invention can be also in IgG4 format. For example four antibodies were generated in IgG4 format, purified, and characterized. All antibodies were capable of binding to human CCR6-, Cyno CCR6-, and mouse CCR6-expressing cells and displayed virtually no binding to cells that do not express the CCR6 orthologs.

### Amino Acid Sequences of Heavy and Light Chains of IgG4 Anti-CCR6

TABLE 34
R605 RC IgG4

EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCARNS
MKGQRKQRGMDYWGQGTLVTVSSASTKGPSVFPLAPCSRSTSESTAALGC
LVKDYPPEPVTVSWNSGALTSGVHTFPAVLQSSGLYSLSSVVTVPSSSLG
TKTYTCNVDHKPSNTKVDKRVESKYGPPCPSCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSPGK
SEQ ID NO. 263

TABLE 35
R605 LC IgG4

EIVLTQSPGTLSLSPGERATLSCRASQSVSSSYLAWYQQKPGQAPRLLIY
GASSRATGIPDRFSGSGSGTDFTLTISRLEPEDFAVYYCQQSRATPLTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC SEQ ID NO. 264

TABLE 36
R606 HC IgG4

EVQLLESGGGLVQPGGSLRLSCAASGFTFSNYYIHWVRQAPGKGLEWVSI
INPWSGRTYYADSVKGRFTISRDNSKNTLYLQMNSLRAEDTAVYYCARGR
LRSLSYSGIDYWGQGTLVTVSSASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSPGK
SEQ ID NO. 265

TABLE 37
R606 LC IfG4

EIVLTQSPGTLSLSPGERATLSCRASQSVSSSYLAWYQQKPGQAPRLLIY
GASSRATGIPDRFSGSGSGTDFTLTISRLEPEDFAVYYCQQSSYSPITFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC SEQ ID NO. 266

TABLE 38
R608 HC IgG4

QVQLVQSGAEVKKPGSSVKVSCKASGGTLTSYYISWVRQAPGQGLEWMGY
ISPSSGYTYYAQKFQGRVTITADESTSTAYMELSSLRSEDTAVYYCARRY
RGRGYVYGLDYWGQGTLVTVSSASTKGPSVFPLAPCSRSTSESTAALGCL
VKDYFPEPVTVSWNSGALTSGVHTFPAVLQSSGLYSLSSVVTVPSSSLGT
KTYTCNVDHKPSNTKVDKRVESKYGPPCPSCPAPEFLGGPSVFLFPPKPK
DTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVL
DSDGSFFLYSRLTVDKSRWQEGNVFSCSVMHEALHNHYTQKSLSLSPGK
SEQ ID NO. 267

TABLE 39
R608 LC IgG4

EIVLTQSPGTLSLSPGERATLSCRASQSVSSSYLAWYQQKPGQAPRLLIY
GASSRATGIPDRFSGSGSGTDFTLTISRLEPEDFAVYYCQQSYYAPVTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC SEQ ID NO. 268

TABLE 40
R623 HC IgG4

EVQLLESGGGLVQPGGSLRLSCAASGFTFSNYYIHWVRQAPGKGLEWVST
IGSSNGTTYYADSVKGRFTISRDNSKNTLYLQMNSLRAEDTAVYYCARGR
AVKLAYYSGFDYWGQGTLVTVSSASTKGPSVFPLAPCSRSTSESTAALGC
LVKDYFPEPVTVSWNSGATLSGVHTFPAVLQSSGLYSLSSVVTVPSSSLG
TKTYTCNVDHKPSNTKVDKRVESDYGPPCPSCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSPGK
SEQ ID NO. 269

TABLE 41
R623 LC IgG4

EIVLTQSPGTLSLSPGERATLSCRASQSVSSSYLAWYQQKPGQAPRLLIY
GASSRATGIPDRFSGSGSGTDFTLTISRLEPEDFAVYYCQQYSYSPITFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC SEQ ID NO. 270

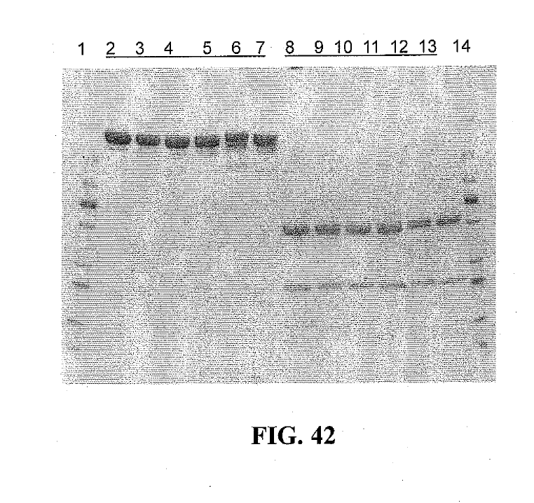

To determine whether Anti CCR6 antibodies in IgG4 format of this invention have the predicted molecular size, we ran SDS-PAGE. Results are shown in FIG. 42. FIG. 42 depicts SDS-PAGE under non-reducing (lanes 1-7) and reducing conditions (lanes 8-14) of the Antibodies of this invention in IgG4 format; respectively, MSM-R605 (lanes 2 and 8); R606 (3 and 9); R625 (4 and 10); R628 (5 and 11); R624 (6 and 12); also analyzed—a prior art IgG4 antibody, Rituximab (7 and 13); molecular weight markers were loaded onto the lanes 1 and 14; 3 microgram IgG4/l e.

As can be seen in FIG. 42, all IgG4 antibodies of this invention were highly homogeneous and did not show any sign of proteolysis and were comparable or better than Rituximab.

### INDUSTRIAL APPLICATION

Fully human antibodies against human CCR6 can be used to detect the presence of CCR6 on cells, and therefore can be used to diagnose disorders involving CCR6. Further, antibodies of this invention can be useful for treating disorders involving CCR6 by inhibiting binding of native chemokines to the CCR6, and thereby decrease effects of those chemokines, or by specific killing cells expressing CCR6 thereby decrease effects of these cells.

1. A fully human antibody against human chemokine receptor CCR6.

2-4. (canceled)

5. The antibody of claim 1, further comprising a pharmaceutically acceptable carrier or excipient.

6. The antibody of claim 1, wherein said antibody is essentially free of contaminants.

7. The fully human anti-CCR6 antibody of claim 1, having a CDR3 HC sequence or a CDR3 LC sequence selected from any of Tables 3 through 41.

8. The fully human anti-CCR6 antibody of claim 1, having a CDR3 sequence selected from Tables 3 through 41.

9. The antibody of claim 1, said antibody in IgG1 format.

10. The antibody of claim 1, said antibody in IgG4 format.

11. An antibody fragment, comprising an scFv or Fab fragment of an antibody of claim 1, where said antibody fragment specifically binds to human CCR6 with an affinity of between about 1 nM to about 100 nM.

12. The antibody of claim 1, further comprising a physiologically compatible solution.

13. The antibody of claim 12, further comprising one or more physiologically compatible excipients or binders.

14. A method for inhibiting an abnormal effect of CCR6, comprising:
administering to a mammal in need thereof an antibody of claim 1.

15. The method of claim 14, wherein said fully human antibody against CCR6 is an antibody fragment.

16. The method of claim 15, where said antibody fragment is an scFv fragment or a Fab fragment of said antibody having a binding affinity of about 1 nM to about 100 nM.

17. (canceled)

18. The method of claim 15, where said antibody or fragment thereof is selected from any of Tables 3-41.

19. The method of claim 18, where the abnormal effect of CCR6 is an abnormal inflammatory response.

20. The method of claim 18, where said abnormal effect is a fibrotic disease, inflammation, or infection.

21. The method of claim 20, where said abnormal effect is a pulmonary disease.

22. The use method of claim 21, where said pulmonary disease is Respiratory Syncytial Virus (“RSV”) induced fibrosis, Idiopathic Pulmonary Fibrosis (“IPF”), Chronic Obstructive Pulmonary Disease (“COPD”), severe Asthma, Fibrotic Sequellae of Acute Lung Injury, or Adult Respiratory Distress Syndrome (ARDS).

23. The method of claim 17, where said abnormality is cancer.

24-28. (canceled)
